SEQUENCE LISTING (10 sequences)

SEQ ID NO: 1 - DNA, 4654 bp, Homo sapiens
gggcgctgctgggctgcggcggcggcggcggcggcggtggttactatggcggagtcggccggagcctcctccttcttcccccttgttgtcctcctgctcgccggcagcggcgggtccgggccccggggggtccaggctctgctgtgtgcgtgcaccagctgcctccaggccaactacacgtgtgagacagatggggcctgcatggtttccattttcaatctggatgggatggagcaccatgtgcgcacctgcatccccaaagtggagctggtccctgccgggaagcccttctactgcctgagctcggaggacctgcgcaacacccactgctgctacactgactactgcaacaggatcgacttgagggtgcccagtggtcacctcaaggagcctgagcacccgtccatgtggggcccggtggagctggtaggcatcatcgccggcccggtgttcctcctgttcctcatcatcatcattgttttccttgtcattaactatcatcagcgtgtctatcacaaccgccagagactggacatggaagatccctcatgtgagatgtgtctctccaaagacaagacgctccaggatcttgtctacgatctctccacctcagggtctggctcagggttacccctctttgtccagcgcacagtggcccgaaccatcgttttacaagagattattggcaagggtcggtttggggaagtatggcggggccgctggaggggtggtgatgtggctgtgaaaatattctcttctcgtgaagaacggtcttggttcagggaagcagagatataccagacggtcatgctgcgccatgaaaacatccttggatttattgctgctgacaataaagcagactgctcattcctcacattgccatgggaagttgtaatggtctctgctgcccccaagctgaggagccttagactccaatacaagggaggaaggggaagagcaagatttttattcccactgaataatggcacctggacacagctgtggcttgtttctgactatcatgagcacgggtccctgtttgattatctgaaccggtacacagtgacaattgaggggatgattaagctggccttgtctgctgctagtgggctggcacacctgcacatggagatcgtgggcacccaagggaagcctggaattgctcatcgagacttaaagtcaaagaacattctggtgaagaaaaatggcatgtgtgccatagcagacctgggcctggctgtccgtcatgatgcagtcactgacaccattgacattgccccgaatcagagggtggggaccaaacgatacatggcccctgaagtacttgatgaaaccattaatatgaaacactttgactcctttaaatgtgctgatatttatgccctcgggcttgtatattgggagattgctcgaagatgcaattctggaggagtccatgaagaatatcagctgccatattacgacttagtgccctctgacccttccattgaggaaatgcgaaaggttgtatgtgatcagaagctgcgtcccaacatccccaactggtggcagagttatgaggcactgcgggtgatggggaagatgatgcgagagtgttggtatgccaacggcgcagcccgcctgacggccctgcgcatcaagaagaccctctcccagctcagcgtgcaggaagacgtgaagatctaactgctccctctctccacacggagctcctggcagcgagaactacgcacagctgccgcgttgagcgtacgatggaggcctacctctcgtttctgcccagccctctgtggccaggagccctggcccgcaagagggacagagcccgggagagactcgctcactcccatgttgggtttgagacagacaccttttctatttacctcctaatggcatggagactctgagagcgaattgtgtggagaactcagtgccacacctcgaactggttgtagtgggaagtcccgcgaaacccggtgcatctggcacgtggccaggagccatgacaggggcgcttgggaggggccggaggaaccgaggtgttgccagtgctaagctgccctgagggtttccttcggggaccagcccacagcacaccaaggtggcccggaagaaccagaagtgcagcccctctcacaggcagctctgagccgcgctttcccctcctccctgggatggacgctgccgggagactgccagtggagacggaatctgccgctttgtctgtccagccgtgtgtgcatgtgccgaggtgcgtcccccgttgtgcctggttcgtgccatgcccttacacgtgcgtgtgagtgtgtgtgtgtgtctgtaggtgcgcacttacctgcttgagctttctgtgcatgtgcaggtcgggggtgtggtcgtcatgctgtccgtgcttgctggtgcctcttttcagtagtgagcagcatctagtttccctggtgcccttccctggaggtctctccctcccccagagcccctcatgccacagtggtactctgtgtctggcaggctactctgcccaccccagcatcagcacagctctcctcctccatctcagactgtggaaccaaagctggcccagttgtccatgacaaaagaggcttttgggccaaaatgtgagggtggtgggtgggatgggcagggaaggaatcctggtggaagtcttgggtgttagtgtcagccatgggaaatgagccagcccaagggcatcatcctcagcagcatcgaggaagggccgaggaatgtgaagccagatctcgggactcagattggaatgttacatctgtctttcatctcccagatcctggaaacagcagtgtatatttttggtggtggtgggtttggggtggggaagggaagggcgggcaaggagtggggagggagtctggggtgggagggaggcatctgcatgggtcttcttttactggactgtctgatcagggtggagggaaggtgagaggtttgcatccacttcaggagccctactgaagggaacagcctgagccgaacatgttatttaacctgagtatagtatttaacgaagcctagaagcacggctgtgggtggtgatttggtcagcatatcttaggtatataataactttgaagccataacttttaactggagtggtttgatttctttttttaattttattgggagggtttggattttaactttttttaatgttgttaaatattaagtttttgtaaaaggaaaaccatctctgtgattacctctcaatctatttgtttttaaagaaatccctaaaaaaaaaaattatccaattgaacgcacatagctcaatcacactggaaatgtttgtccttgcacctgagcctgttcccactcagcagtgagagttcctctttgccctgaggctcagtctctctcgtattttgtccccacccccaattccttgagtggtttttgctctagggccctttcttgcactgtccagctggttgtaccctctccaggcatttattcaacaaatgtgggtgaagtgcctgctgggtgccaggtgctgggaatacatctgtggacaagacatgcttgggtcctactcctggagcactgtaaaaagagctgattcaagtaagtagatgcctgttttgagaccagaaggtttcataattggttctacgacccttttgagcctagaattattgttcttatataagatcactgaagaaagaggaacccccacaaccccctccacaaagagaccaggggcgggtgatgagacctggggtttagaaccccaggtgagacctcaaatcactgcattcattctgagcccccttcctgtccccaggggaggtgtattgtgtatgtagccttagagcatctctgcctccaacccagcagttctctgccaaagcttgtggaggagggagagccctgtccctgccctcaggctccccagtgctcctggcccttctatttatttgactgattattgcttctttccttgcattaaaggagatcttcccctaacctttgggccaatttactggccactaatttcgtttaaataccattgtgtcattggggggaccgtctttacccctgctgacctcccacctatccgccctgcagcagaaccttggcggtttataggtaatgatggaacttagactcctcttcccagagtcacaagtagcctctgggatctgccaacacacgtccactcccaagccactagcccactccccagttggcccttctgcccttaccccacacacagtccaactcttccacctctggggaagatggagcaggtctttgggaagctcccacacccacctctgccactcttaacactaagtgagagttggggagaaactgaagccgtgtttttggccccccgaggctaaccctgatccatagtgctacctgcacctctggattctggattcacagaccaagtccaagcccgttcttacgtcgccataaaggcccccgaacggcattctcggtacttctgtttgtttttgtacattttattagaaaggactgtaaaatagccacttagacactttacctcttcagtatgcaaatgtaaataaattgtaatataggaaatcttttgttttaatataagaatgagcctgtccaatttctgctgtacattattaaaagttttattcacaga

SEQ ID NO: 2 - protein, 505 aa, Homo sapiens
MAESAGASSFFPLVVLLLAGSGGSGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVELVGIIAGPVFLLFLIIIIVFLVINYHQRVYHNRQRLDMEDPSCEMCLSKDKTLQDLVYDLSTSGSGSGLPLFVQRTVARTIVLQEIIGKGRFGEVWRGRWRGGDVAVKIFSSREERSWFREAEIYQTVMLRHENILGFIAADNKDNGTWTQLWLVSDYHEHGSLFDYLNRYTVTIEGMIKLALSAASGLAHLHMEIVGTQGKPGIAHRDLKSKNILVKKNGMCAIADLGLAVRHDAVTDTIDIAPNQRVGTKRYMAPEVLDETINMKHFDSFKCADIYALGLVYWEIARRCNSGGVHEEYQLPYYDLVPSDPSIEEMRKVVCDQKLRPNIPNWWQSYEALRVMGKMMRECWYANGAARLTALRIKKTLSQLSVQEDVKI

SEQ ID NO: 3 - DNA, 6261 bp, Homo sapiens
ggcggctagggaggtggggcgaggcgaggtttgctggggtgaggcagcggcgcggccgggccgggccgggccacaggcggtggcggcgggaccatggaggcggcggtcgctgctccgcgtccccggctgctcctcctcgtgctggcggcggcggcggcggcggcggcggcgctgctcccgggggcgacggcgttacagtgtttctgccacctctgtacaaaagacaattttacttgtgtgacagatgggctctgctttgtctctgtcacagagaccacagacaaagttatacacaacagcatgtgtatagctgaaattgacttaattcctcgagataggccgtttgtatgtgcaccctcttcaaaaactgggtctgtgactacaacatattgctgcaatcaggaccattgcaataaaatagaacttccaactactggtttaccattgcttgttcagagaacaattgcgagaactattgtgttacaagaaagcattggcaaaggtcgatttggagaagtttggagaggaaagtggcggggagaagaagttgctgttaagatattctcctctagagaagaacgttcgtggttccgtgaggcagagatttatcaaactgtaatgttacgtcatgaaaacatcctgggatttatagcagcagacaataaagacaatggtacttggactcagctctggttggtgtcagattatcatgagcatggatccctttttgattacttaaacagatacacagttactgtggaaggaatgataaaacttgctctgtccacggcgagcggtcttgcccatcttcacatggagattgttggtacccaaggaaagccagccattgctcatagagatttgaaatcaaagaatatcttggtaaagaagaatggaacttgctgtattgcagacttaggactggcagtaagacatgattcagccacagataccattgatattgctccaaaccacagagtgggaacaaaaaggtacatggcccctgaagttctcgatgattccataaatatgaaacattttgaatccttcaaacgtgctgacatctatgcaatgggcttagtattctgggaaattgctcgacgatgttccattggtggaattcatgaagattaccaactgccttattatgatcttgtaccttctgacccatcagttgaagaaatgagaaaagttgtttgtgaacagaagttaaggccaaatatcccaaacagatggcagagctgtgaagccttgagagtaatggctaaaattatgagagaatgttggtatgccaatggagcagctaggcttacagcattgcggattaagaaaacattatcgcaactcagtcaacaggaaggcatcaaaatgtaattctacagctttgcctgaactctccttttttcttcagatctgctcctgggttttaatttgggaggtcaattgttctacctcactgagagggaacagaaggatattgcttccttttgcagcagtgtaataaagtcaattaaaaacttcccaggatttctttggacccaggaaacagccatgtgggtcctttctgtgcactatgaacgcttctttcccaggacagaaaatgtgtagtctacctttattttttattaacaaaacttgttttttaaaaagatgattgctggtcttaactttaggtaactctgctgtgctggagatcatctttaagggcaaaggagttggattgctgaattacaatgaaacatgtcttattactaaagaaagtgatttactcctggttagtacattctcagaggattctgaaccactagagtttccttgattcagactttgaatgtactgttctatagtttttcaggatcttaaaactaacacttataaaactcttatcttgagtctaaaaatgacctcatatagtagtgaggaacataattcatgcaattgtattttgtatactattattgttctttcacttattcagaacattacatgccttcaaaatgggattgtactataccagtaagtgccacttctgtgtctttctaatggaaatgagtagaattgctgaaagtctctatgttaaaacctatagtgtttgaattcaaaaagcttatttatctgggtaacccaaactttttctgttttgtttttggaagggtttttgtggtatgtcatttggtattctattctgaaaatgcctttctcctaccaaaatgtgcttaagccactaaagaaatgaagtggcattaattagtaaattattagcatggtcatgtttgaatattctcacatcaagcttttgcattttaattgtgttgtctaagtatacttttaaaaaatcaagtggcactctagatgcttatagtactttaatatttgtagcatacagactaatttttctaaaagggaaagtctgtctagctgcttgtgaaaagttatgtggtattctgtaagccatttttttctttatctgttcaaagacttattttttaagacatgaattacatttaaaattagaatatggttaatattaaataataggcctttttctaggaaggcgaaggtagttaataatttgaatagataacagatgtgcaagaaagtcacatttgttatgtatgtaggagtaaacgttcggtggatcctctgtctttgtaactgaggttagagctagtgtggttttgaggtctcactacactttgaggaaggcagcttttaattcagtgtttccttatgtgtgcgtacattgcaactgcttacatgtaatttatgtaatgcattcagtgcacccttgttacttgggagaggtggtagctaaagaacattctgagtataggtttttctccatttacagatgtctttggtcaaatattgaaagcaaacttgtcatggtcttcttacattaagttgaaactagcttataataactggtttttacttccaatgctatgaagtctctgcagggcttttacagttttcgaagtccttttatcactgtgatcttattctgaggggagaaaaaactatcatagctctgaggcaagacttcgactttatagtgctatcagttccccgatacagggtcagagtaacccatacagtattttggtcaggaagagaaagtggccatttacactgaatgagttgcattctgataatgtcttatctcttatacgtagaataaatttgaaagactatttgatcttaaaaccaaagtaattttagaatgagtgacatattacataggaatttagtgtcaatttcatgtgtttaaaaacatcatgggaaaaatgcttagaggttactattttgactacaaagttgagtttttttctgtagttaccataatttcattgaagcaaatgaatgagtttgagaggtttgtttttatagttgtgttgtattacttgtttaataataatctctaattctgtgatcaggtactttttttgtgggggttttttttttgtttttttttttttgttgttgtttttgggccatttctaagcctaccagatctgctttatgaaatccaggggaccaatgcattttatcactaaaactatttttatataattttaagaatataccaaaagttgtctgatttaaagttgtaatacatgatttctcactttcatgtaaggttatccacttttgctgaagatattttttattgaatcaaagattgagttacaattatacttttcttacctaagtggataaaatgtacttttgatgaatcagggaatttttttaaagttggagtttagttctaaattgactttacgtattactgcagttaattccttttttggctagggatggtttgataaaccacaattggctgatattgaaaatgaaagaaacttaaaaggtgggatggatcatgattactgtcgataactgcagataaatttgattagagtaataattttgtcatttaaaaacacagttgtttatactgcccatcctaggatgctcaccttccaagattcaacgtggctaaaacatcttctggtaaattgtgcgtccatattcattttgtcagtagccaggagaaatggggatgggggaaatacgacttagtgaggcatagacatccctggtccatcctttctgtctccagctgtttcttggaacctgctctcctgcttgctggtccctgacgcagagaccgttgcctcccccacagccgtttgactgaaggctgctctggagacctagagtaaaacggctgatggaagttgtgggacccacttccatttccttcagtcattagaggtggaagggaggggtctccaagtttggagattgagcagatgaggcttgggatgcccctgctttgacttcagccatggatgaggagtgggatggcagcaaggtggctcctgtggcagtggagttgtgccagaaacagtggccagttgtatcgcctataagacagggtaaggtctgaagagctgagcctgtaattctgctgtaataatgatagtgctcaagaagtgccttgagttggtgtacagtgccatggccatcaagaatcccagatttcaggttttattacaaaatgtaagtggtcacttggcgattttgtagtacatgcatgagttaccttttttctctatgtctgagaactgtcagattaaaacaagatggcaaagagatcgttagagtgcacaacaaaatcactatcccattagacacatcatcaaaagcttatttttattcttgcactggaagaatcgtaagtcaactgtttcttgaccatggcagtgttctggctccaaatggtagtgattccaaataatggttctgttaacactttggcagaaaatgccagctcagatattttgagatactaaggattatctttggacatgtactgcagcttcttgtctctgttttggattactggaatacccatgggccctctcaagagtgctggacttctaggacattaagatgattgtcagtacattaaacttttcaatcccattatgcaatcttgtttgtaaatgtaaacttctaaaaatatggttaataacattcaacctgtttattacaacttaaaaggaacttcagtgaatttgtttttattttttaacaagatttgtgaactgaatatcatgaaccatgttttgatacccctttttcacgttgtgccaacggaatagggtgtttgatatttcttcatatgttaaggagatgcttcaaaatgtcaattgctttaaacttaaattacctctcaagagaccaaggtacatttacctcattgtgtatataatgtttaatatttgtcagagcattctccaggtttgcagttttatttctataaagtatgggtattatgttgctcagttactcaaatggtactgtattgtttatatttgtaccccaaataacatcgtctgtactttctgttttctgtattgtatttgtgcaggattctttaggctttatcagtgtaatctctgccttttaagatatgtacagaaaatgtccatataaatttccattgaagtcgaatgatactgagaagcctgtaaagaggagaaaaaaacataagctgtgtttccccataagtttttttaaattgtatattgtatttgtagtaatattccaaaagaatgtaaataggaaatagaagagtgatgcttatgttaagtcctaacactacagtagaagaatggaagcagtgcaaataaattacatttttcccaagtgccagtggcatattttaaaataaagtgtatacgttggaatgagtcatgccatatgtagttgctgtagatggcaactagaacctttgagttacaagagtctttagaagttttctaaccctgcctagtgcaagttacaatattatagcgtgttcggggagtgccctcctgtctgcaggtgtgtctctgtgcctgggggcttttctccacatgcttaggggtgtgggtcttccattggggcatgatggacctgtctacaggtgatctctgttgcctttgggtcagcacatttgttagtctcctgggggtgaaaacttggcttacaagagaactggaaaaatgatgagatgtggtccccaaacccttgattgactctggggaggggctttgtgaataggattgctctcacattaaagatagttacttcaatttgaaggctggatttagggatttttttttttccttataacaaagacatcaccaggatatgaagcttttgttgaaagttggaaaaaaagtgaaattaaagacattcccagacaaa

SEQ ID NO: 4 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 5 - DNA, 5214 bp, Homo sapiens
gtctgggcttccgaatatgttttatgacggttgattttacaccaggaggtttgtctccgaggaagacccagggaactggatatctagcgagaacttcctccggattccccggcgcctcgggaaaatgggagctgctgcaaagttggcgtttgccgtctttcttatctcctgttcttcaggtgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccctattacaacatcctgctctattccttggtgccacttatgttaattgcggggattgtcatttgtgcattttgggtgtacaggcatcacaagatggcctaccctcctgtacttgttccaactcaagacccaggaccacccccaccttctccattactaggtttgaaaccactgcagttattagaagtgaaagcaaggggaagatttggttgtgtctggaaagcccagttgcttaacgaatatgtggctgtcaaaatatttccaatacaggacaaacagtcatggcaaaatgaatacgaagtctacagtttgcctggaatgaagcatgagaacatattacagttcattggtgcagaaaaacgaggcaccagtgttgatgtggatctttggctgatcacagcatttcatgaaaagggttcactatcagactttcttaaggctaatgtggtctcttggaatgaactgtgtcatattgcagaaaccatggctagaggattggcatatttacatgaggatatacctggcctaaaagatggccacaaacctgccatatctcacagggacatcaaaagtaaaaatgtgctgttgaaaaacaacctgacagcttgcattgctgactttgggttggccttaaaatttgaggctggcaagtctgcaggcgatacccatggacaggttggtacccggaggtacatggctccagaggtattagagggtgctataaacttccaaagggatgcatttttgaggatagatatgtatgccatgggattagtcctatgggaactggcttctcgctgtactgctgcagatggacctgtagatgaatacatgttgccatttgaggaggaaattggccagcatccatctcttgaagacatgcaggaagttgttgtgcataaaaaaaagaggcctgttttaagagattattggcagaaacatgctggaatggcaatgctctgtgaaaccattgaagaatgttgggatcacgacgcagaagccaggttatcagctggatgtgtaggtgaaagaattacccagatgcagagactaacaaatattattaccacagaggacattgtaacagtggtcacaatggtgacaaatgttgactttcctcccaaagaatctagtctatgatggttgcgccatctgtgcacactaagaaatgggactctgaactggagctgctaagctaaagaaactgcttacagtttattttctgtgtaaaatgagtaggatgtctcttggaaatgttaagaaagaagaccctttgttgaaaaatgttgctctgggagacttactgcattgccgacagcacagatgtgaaggacatgagactaagagaaaccttgcaaactctataaagaaacttttgaaaaagtgtacatgaagaatgtagccctctccaaatcaaggatcttttggacctggctaatggagtgtttgaaaactgacatcagatttcttaatgtctgtcagaagacactaattccttaaatgaactactgctattttttttaaatcaaaaacttttcatttcagattttaaaaagggtaacttgtttttattgcatttgctgttgtttctataaatgactattgtaatgccaatatgacacagcttgtgaatgtttagtgtgctgctgttctgtgtacataaagtcatcaaagtggggtacagtaaagaggcttccaagcattactttaacctccctcaacaaggtatacctcagttccacggttgctaaattataaaattgaaaacactaacaaaatttgaataataaatcgatccatgttttgtaacaaattcactgtgttatttaaggaaaaaaaggtaagctatgcttagtgccaacaataagtggccattcgtaaagcagtgttttagcatttcttgtgctggcttgtaatgtagggaaaaaaagtgctgttttttgaaaagatggtgtcatttcccccttcttcccatgttttaaagccccatcttatatccagttcccaaaatttgcatacttacctaagtattttttttaggtgtgctgtgtttggggaatatttgaaaatttaaagcatgatttaaaattttttaaagtgagctgtgacactggaaagctcttcattttatcttttaaaatagagttttttctatttatatatgtaaaattgtagtgtatttcttttcaccaaacagtgtgtgggacattctttatcactgttttaggatcacctcaggaagtgtcgttacccagaattccccactgtctgctatgagacttgtaactttatcactatacttctgcttggtgccatcttgtcagagtaatatttgatgtctgtgatatgtaaagaattatcctaggataaagatattaaactttaagcagatttcagatgttactgctttaaaacaaatcagggataacaaattaaacgtataacttaaaatatgcaatgacatttagaggtaaccaatgttgatataggtagcatagcctagcctcctccccaaaattgcttttacaactaacactgatactaatttaggatagttcatgccttatccttgctaagaaaatggaattgatggtaggcaggtgctaaagtgcttttcaaaacaatattacgttagaatacaattggattcttcctcaaatttatacaggccaaaaagtaaaacattaattttctgaatttccagattaccaatcaattaatcaacaaatagccagtattatgctgtgtatttctgtcaggtcattttaaaatccatgttaattttataaaagaattttttacatgtcactgtcaggagctcactgtgaatgtgttgtcttcaaatggttatttaaccacacagtacactacattttacatatatgtacgtaatctctgggaatagtaaattaattatgttatttataaacaatacataggtcaacagactttaagcagggaggaaaagaagagtaatagcgtctgtgtgctgcagaccattcagaactgtcacgtgtgtccccatggtctcattcattgtattcctagcaattcccttttcaatgttgagttcacctctttatttcacaaagtacttggtctctcaatttcttgatctggttttgcttccatttaaaaactaatcaagaagggaaaatattgagaatgtgcatacaagaaaatcattaatttcctgaagatgaatttctacctgttgtgaacatttaactttctttttaaaagttaaacaaaaataaacaagggatattatgatgaatgtttggcttatgtgagtactagagataaaatttttaaacccagttattcacaatataaaatgttttcaagttagaaaaaatttttagaaatcctgggtattgtatttaactgtagctaaccaattttaaaacttgtattcttttgagaactattattaatagaaaaactttttataagcagtaaaataagaatgttccagtgactacctgtccttatacctagtcttgttaaaactttcttttgcagggtatttagtgtttggtttacagtcagtgcagagtgggcaagttaacagaaagtttgagctagagatactggaaaaaaaaaagatcaaagaatgagaaaaatggtgatccattttggggcaaactgagaccccccaaataactctttcctcatgtgtatggtgctcctcatgactcgtcttgtattttgcctttctgatacccatcagaactgctgctgctctaacttatactctttaccttgcccagatctccgcgtaaggaatgctttatgatcaacttgccataggactgatggattaaccagtgttcggctttatttgaagtctatgccctgcacagctcttgtatgtattttagatgctagaagtttttttagcatgtgatgtgtgattcttgtttgaattctaggtaccttgtgaattccagaaaaagagactgtgcttcacgattgttagtcccatgaacttgcactatctatctttcatggtgatgttttgaaaatacaatcaggaaaaaacccaacacctttggaatttaaaatagaatcatatcatgaaatttaaaaagaatctcttctgttgcatttcctcacccctaagtaacagctacatttaagtaaaatgcaggtggtaggggaaaaaaaaccatggcgagatggtggtttagtggaataaactgattactggtttttttgtttttttttttttttttaaagaaagaagcttcatcacagatactttccagtttctcttttatacttttttgaaagattactttttaggaacatttggtatgatatgcataaaattatttatccatttatgggcaaaatgatacaagtagcatcttgattgaacatcatttacctcagatattcaaccagcagtacgttttttatgcagtctcaacccatatcccatttgttacctctcagaatattggtaagcagttattttcgctttactctgtatttcttgtgttttgggcacaggttattgtactactgtcaaatcgtacttgctattttttctgcaagtatttaacagaaagcttaaaatccccataaaaccccaccttggataagtgattgttaaatattgtacaaataaaatgtatgctatccccattccatccccaagttaaataaaaaaatgaatacgg

SEQ ID NO: 6 - protein, 513 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 7 - DNA, 11782 bp, Homo sapiens
ggctcggctccgtggccgccgctcaggagccattttggactcggttcagctcccctcccccccacccctccccccgttcatggcccctccggactcggcccctgcgcccggggcccgggcccagccccgccgcgctatgcctgagtcgggcgcgccccggcccgtgccccgccgccgccccccggcccccgcgtcgccccggagcccgggccgcagcctgcgccgcccgcagcggccctgagcccggccccgccgaccggcccttggagcccgaacgctgctcggggacgaaggcgcaggaagcgcgcagggaacgagaccgaaggaaggagcgggaaggagagcgcagccgccgcctggccctgcgcgccccgggagcgccgtgcggccctgcccgcgggctccgggtgtgcgcggggcggcgccgcggaacatgacggcgccctgggtggccctcgccctcctctggggatcgctgtgcgccggctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgaacgcttcactcatttgccagaggctgggggcccggaagtcacgtacgagccacccccgacagcccccaccctgctcacggtgctggcctactcactgctgcccatcgggggcctttccctcatcgtcctgctggccttttggatgtaccggcatcgcaagcccccctacggtcatgtggacatccatgaggaccctgggcctccaccaccatcccctctggtgggcctgaagccactgcagctgctggagatcaaggctcgggggcgctttggctgtgtctggaaggcccagctcatgaatgactttgtagctgtcaagatcttcccactccaggacaagcagtcgtggcagagtgaacgggagatcttcagcacacctggcatgaagcacgagaacctgctacagttcattgctgccgagaagcgaggctccaacctcgaagtagagctgtggctcatcacggccttccatgacaagggctccctcacggattacctcaaggggaacatcatcacatggaacgaactgtgtcatgtagcagagacgatgtcacgaggcctctcatacctgcatgaggatgtgccctggtgccgtggcgagggccacaagccgtctattgcccacagggactttaaaagtaagaatgtattgctgaagagcgacctcacagccgtgctggctgactttggcttggctgttcgatttgagccagggaaacctccaggggacacccacggacaggtaggcacgagacggtacatggctcctgaggtgctcgagggagccatcaacttccagagagatgccttcctgcgcattgacatgtatgccatggggttggtgctgtgggagcttgtgtctcgctgcaaggctgcagacggacccgtggatgagtacatgctgccctttgaggaagagattggccagcacccttcgttggaggagctgcaggaggtggtggtgcacaagaagatgaggcccaccattaaagatcactggttgaaacacccgggcctggcccagctttgtgtgaccatcgaggagtgctgggaccatgatgcagaggctcgcttgtccgcgggctgtgtggaggagcgggtgtccctgattcggaggtcggtcaacggcactacctcggactgtctcgtttccctggtgacctctgtcaccaatgtggacctgccccctaaagagtcaagcatctaagcccaggacatgagtgtctgtccagactcagtggatctgaagaaaaaaggaaaaaaagttgtgttttgttttggaaatcccataaaaccaacaaacacataaaatgcagctgctattttaccttgactttttattattattattataattattataattattattattaatattattttttggattggatcagtttttaccagcatattgctctactgtatcacaaacagcggacacgtcagcaggcgttgaggtgctgagctgtggatgcagaaccagcgccatgctgaagagcctcagccacctcctgtcctttgggattcgtttttcccgctttctctttgtttgtcgtctcagaatctgtgacacaaagaaacccatctcctgtcttaggaaacctaatgctgcaaactctacctagaggaacctttgaagactgttacataagaacataccttcctcagaagaggagtttcctctgccctctgcccttctcccctgcctccctccctcccctccttttattttgttttagtgagcttaagaaacagcagatgtgtctttcacggatctaacgggtgttgtcctgatcgagaaaaaaactgggatgagaatggtttggactggagttggaaggggaggacggtactgggggtagggtttggaacagagctacactggactcgggcacattcggagcagcatcctttagtatggaggctacttctcaggtaaccaggaattgaggggaaggaccttgtggaggccgagcattaacagcaagagcggggtttggagaaagtctgagattgggtgcagccctgacttacctgctggccctgaccagtttcttttcactaacttggccttgggcataggatgaaacattttttctgccctaattttaaaactaggtgagggtagaatcatcacaggttaggaatacattcttcataagacacgatgctgtaaatacccttaatggacgaaaagttgaaatacttttgtttcctcttggagcagttcagggaaatgcccacaggggattgtcctgcacagatagggcaagaggatttcctgggtggagtctgccaaggcctgcctcgctggggaccccagagtcctgcacctctggttccgccccaggtggtgacattactgtccccgttctgtggctcgtggacaagactttctccagaccccttaaagtggtacatattctaaaaaactgtttttctattatgccataaccttgctctagtcagtgaatgttcctaatgctgctgtttcaacatttgaattctttttaatttatgaaacatgctaaattttttttttcaaacaaaacacacacatccacatatacacatgcttcgctatgtggcttccaaggtttaaattttgaaaagtaaaagaattaaaacttcacgaccacagatcacctcaaaccagaaatacctcagaattttctacttatgtaaggtttattatatattttgttagttgtgttgtcttgtagtaagtatattttaatgtaagttggcttttgtgacaaggaagtttaaaagaaatagagaaaaagaaaaaagtttgcatcttctagggagtgctaccatttttgtttgataacgcccccttgtaaataattgtcatcaactgtaggttggctgtctgggccaagtctgggcatttatcagtcttgtttgtgaaggcttttccttctggtttctttagatcattttatttaaaaacagtgcatctcttcatcgtgagggtaggcaaggcgggggccgtggggagaggttgacctgggtgagaactgaagaggccgcctcctcttgggttgtttggagcttcacatgtaattcacatgtaacatgtaacttgatcggtcagtgttcagaatgacaagtaaccccgcttaaacttggtagaaggatggcccttagacctgaatggggtgattttacttgggatttaacttcttcagcaaattaacagcaacgttggaagagatctgtggcgcctctgtgaagcacaccgtgactcaggccagtcttttagtgcagcgtgtctgggagtgaagggttttgcccttgctggtcttggagtccacagtgtgaggggcactgcacatgcctgggcatctacctagtgtgctatgttcagtgtctggggcttactgccccggggtcctttcctctgggtgttggggcacagggtgctatgggaggcccatttgcttccctctcggagctcagtttttgcttcatgggtcaaaatgtgggctggccaagtggttacaggaacagggtttcggtaagctatgttgtctttttttttttttttttttttttttttaatggtttgattttgtgctgtggtattttttttcccttagaataatttttaatggcaaaacaggccttacagcagttgcttttctttaccatttatttctttaagaagctttaaaatatttattgaaaagtgccatatctaatttctttagctttcgcctcaggcagtgcaggcatctttacttttcatcctcagaagaaacaaacgactaacaaatgtagcaaatttactgcaggaatagttaggtcatgatactacctgaacactaaaccccagcctctttgtttggttttagttcctctgggtggtttttcttttgtgtgctggcttgattcttgtgagaagttttgacctggccaagggagggttgagccatggttctggtgtgggactttgcggtcaagacacagtacagacaggtcaggcctgcgtgccttttctctgggtggcctccccgttaggcccaccgtacgctcagccactatagtgtccctgtggggccttgccatcagattgtgtgtcaggagatggtacctttttggtgtggctggggaggagtgtggtccatgccagttctttgggcttcaggccactcttcccctcatgctgtggtgtaaagtgcacccatcaggtggtatatctggttctgatggcaagaagaaggtgggggatctccttatagggcatgggtctaggagcacagatgggccttttgccccgggtaaatgcttgtctgtttgctgtcatgtgttctttgaggagtgagccatctcgagccctgctttgaatttactgggtcatagagcctctgcctgtgctcttttccataatgacttcatgtgacatgcacttttggtgggctcagataattggtttctttttgtttttgacctcaggctctgtggcagactggggaaaatggggcctggcatcattttccctgtcaatgggaggggctgttccatgcagggtgggaggggaccaagttagcagagagtagccaaggatccttgcttcttcctttctagtgtgctgtcatccaagcaggctcctggctgtagggatgggccttggggaagaatcttctttgaaagcatctatgataactgagaagtcatccctagttggagaaatccagtaatgagcagaaggaggaagcaagtgaggacagaggccattgtattacagtgtcacgcagagggccctcaatgatggggcattggggaaggctgtagacatagtcatcagaacatcctggcctggcataagctgggttttctcctgggaccattggtcctcagcaggagttctttgcatgagttgctcaggggcaagggctgcaagtgggctgtgcttaggagaaagtgacacctggcagtgagggaagatggtgagcattattagcctttgttgtccagcatggccttcttgtcctgtctgctctggagaggagcctgtgggaccagtcctgcctggggagggcatacccacacgtgccagctgattctgactctgaatacatcatgtccggacttgggggtgtttctgcagaaaaaggaggttgtttttcagccttgaacatcttcaggaggatagagactcttgctcacatattcttagcaaagggaagggtctctcatctccaggccacagagatagttcttccattgccctaagaggctaggctaaccctcttgacataacttagacagcaaagcacttcatcctgtagttgggctctgtcacctttctcttcagttggccacattctcgtttcctccatcctgctatgctttgtgtgctcgggctgtgtgtggggtttttccctggtggaaggaagcccagctgtgtattgaatgtccttcatgtgttgtgtgtggctcagaaagcctgtcacttggcccctgtgctctgagccgtgagggtggggaggtggctgttccattaaagtgggagtattggatggccctcttgaaactagaattttgccttttttagtatgcagtataaagtttccagcatctattggtaacacaaagatttgctggtttttaaaataatacagtaagcataagtatgtaagtttttagaattggtactagaagttggacagctagttattctcgagaactttatttcactagaaaaatatactaattggaaagcagtttccaggagttaactcagtttaattttcagtctcagttattttagcctgttgagtttttgatggcacacctttggagagatggccacgcctgattcccatttcaggggcatcagaccatacctttttaagaagctccgtgaatctagtcatctacccttcatcctgggcgaacagccaaaaagagaaggggacaaggtgtctttttctccttctcactggggtgacatgaattcttttagttaatggctgtttgcaaattctaaactaatgaaatacttagcagctaacatgttcaatctagtaatgatgagtttaaatctcaattgacagtaatgttttagataaacaggcccagtaattcagttgatgaactgtatatcttctcagtctagatttgtaaatgtttaatgaattcagggttataagcatagttctttaagtaagattccagatagttgatttgcaaccagcagtctacctatgaatgtatcccaaacctttagaagattggaaaagatttttgaaataatgatttagttttgtaggaaaaacacccccttgaaaattaattcggttgacccagtaacattttttaaaacaattggtggctccaaaaggcctgccaacaaagaaaagtccaaattatctagtgggacattttgaatgttttatgtttattttgggtccactgtaaactttggttcaaaaaagaatttgaatttaaagaatttaccattatttaaattattaccaagtttttacattttcatgatggtattttccaggtatgaatgaaacatgactttttgattgtggtacttcctgtatcccctgtagtgccaaaaccagtgatactttatttgctcctatggcagctcatagaggtaaccgaagtgatttttcctcagtaattgaaacacatattctctaaatgccaatgtgtggtgatgggccctgcactgccttcatttctctagggcagtgtctttggattgtctagggcctaggtaattctgagaactactgtaaaccaaccacagggcactaaagcaatgtacacaccactctttgtgtgtatggaaggggttatataaacctgggctatgctggacatctacagaagagtattacattcacttgcaaagtttacatttttgagctcacagttatgaaaaatatgacccacaagtttttcaggcaggtgaggatgggtcttcttgcaaatgcatgagttctgtcttgagtcctgggaacttctctgttggttgagtgtgggctcattccctgactctcctaatcatgtttgcgtcagaatgttagcattgtaaataaaagaataggttgtataatagatacacaacacttgaaactttactttaaaaaaatcgatagttctacatatatatttagttatatcacttgacagatttcttctacacagtgtggagattgttttataccacagattatttttataaagttagtgaatttgaatgattttgtaatcagagctaatgagctttacctttcaagagaaacgtacactggagcatgagtggtgtggaacttttacttagtgtttatatggattcttgtgatacactggcagactggagtcaatttgcgggtcttttttggccaaaactccacttgtggttgtgtaggacagtgatattcagctcagcttcttgtggattgggaggagagagggcctgcaatgtgttttacattggtgcttcctcctgagatttctgttgaacaaagggttctgaggtcaaaaattagtttgtaagcctttgccataggacatagtcatgtgagagtgtttgggggaacagaaattgtataggggtgcctattggggtgggatgggactcgaataagattcaggtacaaaaactttgaaatgagaatctggtggtttgagtaatccaccagactgaattatctaagatcacattatccaggttggggggcagaattacccagttaagtaattgttcagaaaagtggggagggtggcatgtggatgcagtgatccaattaaatggagagctgccaggcacattttgtcctctctggtcagtgagaatggttgggttggctcgctgcttcaatctgtggaatcagccaggagcccagtgaggaagctcagaaccccagtaacagcagagcatctttcagatagctccagagttttcctgcttttctgaggaagctcagcatcactgccacaatacggaaagtggtcttcattttagcctatttatttttaggcagagagtggatggttatttgtgtgggacttttggtggcgatatataatgaataattaagttaatttctggtatgcataatggccagtcctgaggcccagctgaagacctgtcccccagaccctgcccgctggcttcaggctgctgcttctagacagaggtgcactggacgggatagttttatcaagagaatccctaatgtgtcattttaaaccagctgtgctttttattcattctggttgagcgtataggtttacactttaccctttttatacttggaataaatttagttccagcagatctagtagcactccagaaaccaaccccatctgttccccataaaaagaacattttctctgctctccagccacgtgtcttggaatgtaattctgttgtgcctttgtttttatcactctcttcgccccaaaagcaactgctgtaagcttttttctacttgtcttttctagtccccaacctctacctttttcctttttcccagccctaatttctggatgcacttctgtgatccaggtattttaagaaccagttacctcagacctcatgttgaacagtgtcgccatctgggtcctcttgatactgcagacttttaacgtacacatgcaggaaccctgctgagcgtgggcacttgttttaaagcaaaactcttcccaaggactgaagaaagggcttctggcaagctcgtcatggcattgtggtgggatgggtctagagtgtcatctgaatggtgcttcctgtgttcctctttgaattctgccattttcagtattcttgtgtgtctgaataggcaaagcgatttaattggctggtcttgcacgcaaattagttccaaagataagctctttgtaacacatttccagtcgctaatgctcaaatgtagaacattcctttaaatggcaggataaaaaacccactatccaccatagtgcattttgggaagatgtctgtagcatatgttgctgtgaaattaggccttgtgggatatggctgtttgtcattttgatgtattttaaataaatatatatattttttaaagagccttttttaccagttcaaaaagtttaattaaccagcagtcaccgcatctgaatttttgtctctggggcatagatggcagaccaagattaaaagtggtaactcagctatacgagcatgggctaccttcctgggctctcctgcagtcctgtagacctgctgttccgcagaccatgggacacaaggtcagtgtgttcccagtgagggtcccaagtcagtcatcttaagtgtttgttctctgccccattcagtggactgttgacttcagtccctgcaagtgctttagcccgagtggggttttctcagagcactgccacgagttaagtgtgtgtttagccaaataatttctccgtaagggaaaaatgcagtcacccaaattttaccaacaatgacagagatgagagtagaaaagattaggcaacatctgagttttaacttgaaaagtgtccaagtcatcatgaaaggccgactgggagcaagtgattattagagattcttcaggagacctcatctgaaaatgttaagactgccagtgagggaaggaattgttaaaatgccagcggcttttttttcctctttttttctgtaattctgtaaaaatgcagagaaagttgagtggtacttcagaattgagggagagggttaccgcagagtagaaatatatttctagatttcagttccacaccacaaatccacaacaatgccatttttcaactgtacaaaaatctgcttatgaactggacatgatcttaatggtagtgtcaaaggccaagtttttcacctgttaatatttttccacatttgtccttgaatctgaataactttatacagtactgtaaatttaacttacatcgagtttgttgtcaattcttatgaaaagagctttctgcatgtaacacatacggttaaagaacacagcaaaggacaaaatttgcaggaacagttttggaaccaacagaaaatgtcaccttttatttgccatcttatatatatctatcagttttaccagctacttctaaatttgtacattatttgtaagggaaagaaggaaaaccctaagacttgtctaacttagtggagaatgtgtgtgttgggcttaggatggatagctaagtcttattgagctgtgttacctaacttgtatataaaaattgtaattaaaagtttgggttcacctgtttctcacagtttaaaatgatgagtaattgcaaactctggaaatgtgactagtatatgatttaaggctgtagaagcaaggaagctctttcaagtgctaaaactaaagacttctagtttttggctcaaataagtactgtttgtataccaggatatgtgagatgtaaatgtagtaggtcacttttcacccttgtagctataaaataaaaattttgtagaacagaaatagcttgtactactgaattaacaaaagttatactaaagtatcatgtttaaaaaaaatatatatatatatacagagttaagcttgttgctgttaccctgtctggatttgaaaagtgtgctgatttatatatatatattacacacacacacacacacacacacacacacacacacacacacacacacacacacacacacacacacacacacatacacctaaaatggcctaaagcagacatccatgtaattacagttgcaaaatgaaaacattttggaaagaacattgtatcatagttcattcatttgcagtggatctttgttcctttttactgtggtaattttagaaatgagtgtcaagtttgaaattagatctgctaagttggggttttgctgcttgaactctgcactgggtcctcaaataaaccgatgtgaatgtagttttttccccctgtgtgaagaagcagttacaccccaacaataggaggaaaaatctagaactatttcaagttttatctttttgtatatgaaaataaaataataataaaacaa

SEQ ID NO: 8 - protein, 512 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 9 - DNA, 4704 bp, Homo sapiens
ggagagggagaaggctctcgggcggagagaggtcctgcccagctgttggcgaggagtttcctgtttcccccgcagcgctgagttgaagttgagtgagtcactcgcgcgcacggagcgacgacacccccgcgcgtgcacccgctcgggacaggagccggactcctgtgcagcttccctcggccgccgggggcctccccgcgcctcgccggcctccaggccccctcctggctggcgagcgggcgccacatctggcccgcacatctgcgctgccggcccggcgcggggtccggagagggcgcggcgcggaggcgcagccaggggtccgggaaggcgccgtccgctgcgctgggggctcggtctatgacgagcagcggggtctgccatgggtcgggggctgctcaggggcctgtggccgctgcacatcgtcctgtggacgcgtatcgccagcacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtctgtggatgacctggctaacagtgggcaggtgggaactgcaagatacatggctccagaagtcctagaatccaggatgaatttggagaatgttgagtccttcaagcagaccgatgtctactccatggctctggtgctctgggaaatgacatctcgctgtaatgcagtgggagaagtaaaagattatgagcctccatttggttccaaggtgcgggagcacccctgtgtcgaaagcatgaaggacaacgtgttgagagatcgagggcgaccagaaattcccagcttctggctcaaccaccagggcatccagatggtgtgtgagacgttgactgagtgctgggaccacgacccagaggcccgtctcacagcccagtgtgtggcagaacgcttcagtgagctggagcatctggacaggctctcggggaggagctgctcggaggagaagattcctgaagacggctccctaaacactaccaaatagctcttctggggcaggctgggccatgtccaaagaggctgcccctctcaccaaagaacagaggcagcaggaagctgcccctgaactgatgcttcctggaaaaccaagggggtcactcccctccctgtaagctgtggggataagcagaaacaacagcagcagggagtgggtgacatagagcattctatgcctttgacattgtcataggataagctgtgttagcacttcctcaggaaatgagattgatttttacaatagccaataacatttgcactttattaatgcctgtatataaatatgaatagctatgttttatatatatatatatatatctatatatgtctatagctctatatatatagccataccttgaaaagagacaaggaaaaacatcaaatattcccaggaaattggttttattggagaactccagaaccaagcagagaaggaagggacccatgacagcattagcatttgacaatcacacatgcagtggttctctgactgtaaaacagtgaactttgcatgaggaaagaggctccatgtctcacagccagctatgaccacattgcacttgcttttgcaaaataatcattccctgcctagcacttctcttctggccatggaactaagtacagtggcactgtttgaggaccagtgttcccggggttcctgtgtgcccttatttctcctggacttttcatttaagctccaagccccaaatctggggggctagtttagaaactctccctcaacctagtttagaaactctaccccatctttaataccttgaatgttttgaaccccactttttaccttcatgggttgcagaaaaatcagaacagatgtccccatccatgcgattgccccaccatctactaatgaaaaattgttctttttttcatctttcccctgcacttatgttactattctctgctcccagccttcatccttttctaaaaaggagcaaattctcactctaggctttatcgtgtttactttttcattacacttgacttgattttctagttttctatacaaacaccaatgggttccatctttctgggctcctgattgctcaagcacagtttggcctgatgaagaggatttcaactacacaatactatcattgtcaggactatgacctcaggcactctaaacatatgttttgtttggtcagcacagcgtttcaaaaagtgaagccactttataaatatttggagattttgcaggaaaatctggatccccaggtaaggatagcagatggttttcagttatctccagtccacgttcacaaaatgtgaaggtgtggagacacttacaaagctgcctcacttctcactgtaaacattagctctttccactgcctacctggaccccagtctaggaattaaatctgcacctaaccaaggtcccttgtaagaaatgtccattcaagcagtcattctctgggtatataatatgattttgactaccttatctggtgttaagatttgaagttggccttttattggactaaaggggaactcctttaagggtctcagttagcccaagtttcttttgcttatatgttaatagttttaccctctgcattggagagaggagtgctttactccaagaagctttcctcatggttaccgttctctccatcatgccagccttctcaacctttgcagaaattactagagaggatttgaatgtgggacacaaaggtcccatttgcagttagaaaatttgtgtccacaaggacaagaacaaagtatgagctttaaaactccataggaaacttgttaatcaacaaagaagtgttaatgctgcaagtaatctcttttttaaaactttttgaagctacttattttcagccaaataggaatattagagagggactggtagtgagaatatcagctctgtttggatggtggaaggtctcattttattgagatttttaagatacatgcaaaggtttggaaatagaacctctaggcaccctcctcagtgtgggtgggctgagagttaaagacagtgtggctgcagtagcatagaggcgcctagaaattccacttgcaccgtagggcatgctgataccatcccaatagctgttgcccattgacctctagtggtgagtttctagaatactggtccattcatgagatattcaagattcaagagtattctcacttctgggttatcagcataaactggaatgtagtgtcagaggatactgtggcttgttttgtttatgtttttttttcttattcaagaaaaaagaccaaggaataacattctgtagttcctaaaaatactgacttttttcactactatacataaagggaaagttttattcttttatggaacacttcagctgtactcatgtattaaaataggaatgtgaatgctatatactctttttatatcaaaagtctcaagcacttatttttattctatgcattgtttgtcttttacataaataaaatgtttattagattgaataaagcaaaatactcaggtgagcatcctgcctcctgttcccattcctagtagctaaa

SEQ ID NO: 10 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

(19) United States
(12) Patent Application Publication
(10) Pub. No.: US 2023/0414711 A1
(43) Pub. Date: Dec. 28, 2023

(54) THERAPEUTICS TARGETING TRANSFORMING GROWTH FACTOR BETA FAMILY SIGNALING

(71) Applicants: The Jackson Laboratory, Bar Harbor, ME (US); University of Connecticut, Farmington, CT (US)

(72) Inventors: Se-Jin Lee, Bar Harbor, ME (US); Emily Germain-Lee, Bar Harbor, ME (US)

(73) Assignees: The Jackson Laboratory, Bar Harbor, ME (US); University of Connecticut, Farmington, CT (US)

(21) Appl. No.: 18/036,704

(22) PCT Filed: Nov. 12, 2021
(86) PCT No.: PCT/US2021/059157

§ 371 Date: May 12, 2023

Related U.S. Application Data
(60) Provisional application No. 63/227,232, filed on Jul. 29, 2021.
(60) Provisional application No. 63/113,575, filed on Nov. 13, 2020.

(51) Int. Cl. A61K 38/17; A61P 21/00

(52) U.S. Cl. CPC A61K 38/1719; A61P 21/00

(57) ABSTRACT

Provided herein are methods of inhibiting type 1 receptor and/or type 2 receptor signaling in subjects, optionally subjects with diseases associated with myostatin, type 1 receptor, and/or type 2 receptor signaling, or muscle loss and/or bone deterioration. Also provided are compositions for inhibition of type 1 receptor and/or type 2 receptor signaling.

### RELATED APPLICATIONS

This application claims the benefit under 35 U.S.C. § 119(e) of U.S. provisional application No. 63/113,575 filed Nov. 13, 2020, and U.S. provisional application No. 63/227,232 filed Jul. 29, 2021, each of which is incorporated by reference herein in its entirety.

### GOVERNMENT LICENSE RIGHTS

This invention was made with government support under Grant Nos. R01 AR060636 and R01 AG052962 awarded by National Institutes of Health. The government has certain rights in the invention.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Nov. 4, 2021, is named J022770098WO00-SEQ-NTJ, and is 65,794 bytes in size.

### BACKGROUND

Myostatin (MSTN) is a secreted signaling molecule of the transforming growth factor-β (TGF-β) family that normally acts to limit skeletal muscle and bone growth. Mice lacking MSTN exhibit dramatic increases in muscle mass throughout the body, with individual muscles growing to about twice the normal size. MSTN appears to play two distinct roles in regulating MSTN also appears to play two distinct roles in regulating bone mineral density: one to limit differentiation of osteoblasts, which promote bone deposition, and another to promote the activity of osteoclasts, which promote bone resorption. The sequence of MSTN has been highly conserved through evolution, with the mature MSTN peptide being identical in species as divergent as humans and turkeys, and the function of MSTN has also been conserved. Targeted or naturally occurring mutations in MSTN have been shown to cause increased muscling in numerous species, including cattle, sheep, dogs, rabbits, rats, swine, goats, and humans.

Numerous pharmaceutical and biotechnology companies have developed agents capable of blocking MSTN activity. These agents have been tested in clinical trials for a wide range of indications, including Duchenne and facioscapulohumeral muscular dystrophy, inclusion body myositis, spinal muscular atrophy, muscle atrophy following falls and hip fracture surgery, age-related sarcopenia, Charcot-Marie-Tooth disease, cachexia due to chronic obstructive pulmonary disease, end stage kidney disease, and cancer. However, no MSTN inhibitors have reached drug approval, and the effects of MSTN inhibition on bone growth are unknown. The function of MSTN is partially redundant with that of another TGF-β family member, activin A. MSTN and activin A signal through a complex of type 1 and type 2 receptors.

### SUMMARY

The present disclosure provides, in some aspects, compositions comprising inhibitors of type 2 receptors (activin type 2 and/or TGFβ type 2) or specific combinations of type 1 (activin type 1 and/or TGFβ type I) and/or type 2 receptors, which respond to myostatin signaling by limiting skeletal muscle and bone growth. Inhibiting myostatin signaling by blocking the activity of type 1 and/or type 2 receptors, for example, promotes skeletal muscle and bone growth. Inhibiting specific combinations of receptors, as described herein, provides key benefits over current methods, for example, those that target only type 1 receptors or those that target individual receptors. As demonstrated herein, functional redundancy exists not only among type 1 receptors but also among type 2 receptors (such that myostatin can signal through one type of a receptor if another type is unavailable). This mechanism renders inhibition of a single receptor insufficient to prevent myostatin signaling. Inhibition of specific combinations of type 1 and/or type 2 receptors overcomes this problem of functional redundancy, effectively preventing myostatin signaling. Surprisingly, however, the data provided herein demonstrates that inhibition of certain combinations of receptors (e.g., ALK5 and ACVR2A) has a far greater impact on muscle growth, relative to inhibition of certain other combinations. These findings suggest that targeted inhibition of these receptors is a promising therapy for diseases associated with muscle loss.

Some aspects of the present disclosure provide a method of increasing muscle weight in a subject, comprising administering to the subject an agent or a combination of agents that inhibit(s) ALK4 and/or ALK5 signaling in the subject.

Other aspects of the present disclosure provide a method of reducing body fat content in a subject, comprising administering to the subject an agent or a combination of agents that inhibit(s) ALK4 and/or ALK5 signaling in the subject.

Yet other aspects of the present disclosure provide a method of improving glucose metabolism in a subject, comprising administering to the subject an agent or a combination of agents that inhibit(s) ALK4 and/or ALK5 signaling in the subject.

In some embodiments, a single agent is administered. The single agent may inhibit, for example, ALK4 and ALK5 signaling (e.g., may bind to both ALK4 and ALK5). In some embodiments, the single agent may specifically inhibit ALK4 and ALK5 signaling. In other embodiments, two (or more, e.g., three or four) agents are administered. For example, one agent may inhibit (e.g., specifically inhibit) ALK4 signaling, while another agent may inhibit ALK5 signaling (e.g., specifically inhibit).

Non-limiting examples of such agents include, antibodies, soluble receptors, small molecules, and other non-peptide molecules, such as antisense oligonucleotides (ASOs), RNA interference (RNAi) molecules, and programmable-nuclease-based gene editing systems.

In some embodiments, the agent or combination of agents is/are administered to the subject in an effective amount to (directly or indirectly) increase muscle weight (also referred to as muscle mass) in the subject by at least 40% relative to a control or baseline. For example, a single agent that inhibits (e.g., specifically inhibits) ALK4 and/or ALK5 signaling (e.g., by binding to ALK4, ALK5, or both ALK4 and ALK5) may be administered to the subject in an effective amount to increase muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline. As another example, two agents, one that inhibits ALK4 signaling (e.g., by binding to ALK4) and one that inhibits ALK5 signaling (e.g., by binding to ALK5) may be administered to the subject in an effective amount to increase muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline. A control may be administration of a placebo (e.g., saline) or baseline (e.g., muscle weight within 24 hours prior to administration of the one or more agent(s))).

In some embodiments, the agent or combination of agents inhibit(s) ALK4 and/or ALK5 signaling by binding to (e.g., specifically binding to) ALK4 and/or ALK5.

In some embodiments, the agent or combination of agents inhibit(s) ALK4 and/or ALK5 signaling specifically in myofibers of the subject. For example, the agent(s) may be selected from ASOs, RNAi molecules (e.g., shRNA, siRNA, or miRNA), and programmable nuclease-based gene editing molecules (e.g., CRISPR/Cas9/gRNAs, TALE/TALENs, and ZFNs) that specifically target ALK4 and/or ALK5.

In some embodiments, the agent or combination of agents is/are administered to the subject in an effective amount to increase tricep muscle weight in the subject by at least 50% relative to a control or baseline. For example, a single agent that inhibits (e.g., specifically inhibits) ALK4 and/or ALK5 signaling (e.g., by binding to ALK4, ALK5, or both ALK4 and ALK5) may be administered to the subject in an effective amount to increase tricep muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline. As another example, two agents, one that inhibits ALK4 signaling (e.g., by binding to ALK4) and one that inhibits ALK5 signaling (e.g., by binding to ALK5) may be administered to the subject in an effective amount to increase tricep muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline.

In some embodiments, the agent or combination of agents is/are administered to the subject in an effective amount to increase quadricep muscle weight in the subject by at least 3% relative to a control or baseline. For example, a single agent that inhibits (e.g., specifically inhibits) ALK4 and/or ALK5 signaling (e.g., by binding to ALK4, ALK5, or both ALK4 and ALK5) may be administered to the subject in an effective amount to increase quadricep muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline. As another example, two agents, one that inhibits ALK4 signaling (e.g., by binding to ALK4) and one that inhibits ALK5 signaling (e.g., by binding to ALK5) may be administered to the subject in an effective amount to increase quadricep muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline.

In some embodiments, the agent or combination of agents is/are administered to the subject in an effective amount to increase gastrocnemius/plantaris muscle weight in the subject by at least 3%, at least 4%, or at least 5% relative to a control or baseline. For example, a single agent that inhibits (e.g., specifically inhibits) ALK4 and/or ALK5 signaling (e.g., by binding to ALK4, ALK5, or both ALK4 and ALK5) may be administered to the subject in an effective amount to increase gastrocnemius/plantaris muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline. As another example, two agents, one that inhibits ALK4 signaling (e.g., by binding to ALK4) and one that inhibits ALK5 signaling (e.g., by binding to ALK5) may be administered to the subject in an effective amount to increase gastrocnemius/plantaris muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline.

Some aspects of the present disclosure provide a method of increasing muscle weight in a subject, comprising administering to the subject an agent or a combination of agents that inhibit(s) ACVR2A and ALK5 signaling in the subject.

Other aspects of the present disclosure provide a method of reducing body fat content in a subject, comprising administering to the subject an agent or a combination of agents that inhibit(s) ACVR2A and ALK5 signaling in the subject.

Yet other aspects of the present disclosure provide a method of improving glucose metabolism in a subject, comprising administering to the subject an agent or a combination of agents that inhibit(s) ACVR2A and ALK5 signaling in the subject.

In some embodiments, a single agent is administered. The single agent may inhibit, for example, ACVR2A and ALK5 signaling (e.g., may bind to both ACVR2A and ALK5). In some embodiments, the single agent may specifically inhibit ACVR2A and ALK5 signaling. In other embodiments, two (or more, e.g., three or four) agents are administered. For example, one agent may inhibit (e.g., specifically inhibit) ACVR2A signaling, while another agent may inhibit (e.g., specifically inhibit) ALK5 signaling.

In some embodiments, the agent or a combination of agents is/are administered to the subject in an effective amount to increase muscle weight in the subject by at least 3%, at least 4%, or at least 5% relative to a control or baseline. For example, a single agent that inhibits (e.g., specifically inhibits) ACVR2A and ALK5 signaling (e.g., by binding to both ACVR2A and ALK5) may be administered to the subject in an effective amount to increase muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline. As another example, two agents, one that inhibits ACVR2A signaling (e.g., by binding to ACVR2A) and one that inhibits ALK5 signaling (e.g., by binding to ALK5) may be administered to the subject in an effective amount to increase muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline.

In some embodiments, the agent or combination of agents inhibit(s) ACVR2A and ALK5 signaling by binding to ACVR2A and ALK5.

In some embodiments, the agent or combination of agents inhibit(s) ACVR2A and ALK5 signaling specifically in myofibers of the subject. For example, the agent(s) may be selected from ASOs, RNAi molecules (e.g., shRNA, siRNA, or miRNA), and programmable nuclease-based gene editing molecules (e.g., CRISPR/Cas9/gRNAs, TALE/TALENs, and ZFNs) that specifically target ACVR2A and/or ALK5.

In some embodiments, the agent or combination of agents is/are administered to the subject in an effective amount to increase tricep muscle weight in the subject by at least 3%, at least 4%, or at least 5% relative to a control or baseline. For example, a single agent that inhibits (e.g., specifically inhibits) ACVR2A and ALK5 signaling (e.g., by binding to both ACVR2A and ALK5) may be administered to the subject in an effective amount to increase tricep muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, or at least 50% relative to a control or baseline. As another example, two agents, one that inhibits ACVR2A signaling (e.g., by binding to ACVR2A) and one that inhibits ALK5 signaling (e.g., by binding to ALK5) may be administered to the subject in an effective amount to increase tricep muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, or at least 50% relative to a control or baseline.

In some embodiments, the agent or combination of agents is/are administered to the subject in an effective amount to increase quadricep muscle weight in the subject by at least 3%, at least 4%, or at least 5% relative to a control or baseline. For example, a single agent that inhibits (e.g., specifically inhibits) ACVR2A and ALK5 signaling (e.g., by binding to both ACVR2A and ALK5) may be administered to the subject in an effective amount to increase quadricep muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, or at least 70% relative to a control or baseline. As another example, two agents, one that inhibits ACVR2A signaling (e.g., by binding to ACVR2A) and one that inhibits ALK5 signaling (e.g., by binding to ALK5) may be administered to the subject in an effective amount to increase quadricep muscle weight in the subject by at least 3%, at least 4%, at least 5%, at least 6%, at least 7%, at least 8%, at least 9%, at least 10%, at least 15%, at least 20%, at least 25% at least 30%, at least 35%, at least 40%, at least 45%, at least 50%, at least 55%, at least 60%, at least 65%, or at least 70% relative to a control or baseline.

In some embodiments, the agent or combination of agents is/are administered to the subject in an effective amount to increase gastrocnemius/plantaris muscle weight in the subject by at least 3%, at least 4%, or at least 5% relative to a control or baseline. For example, a single agent that inhibits (e.g., specifically inhibits) ACVR2A and ALK5 signaling (e.g., by binding to both ACVR2A and ALK5) may be administered to the subject in an effective amount to increase gastrocnemius/plantaris muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, or at least 80% relative to a control or baseline. As another example, two agents, one that inhibits ACVR2A signaling (e.g., by binding to ACVR2A) and one that inhibits ALK5 signaling (e.g., by binding to ALK5) may be administered to the subject in an effective amount to increase gastrocnemius/plantaris muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, or at least 80% relative to a control or baseline.

Some aspects of the present disclosure provide a method of increasing muscle weight in a subject, comprising administering to the subject an agent or a combination of agents that inhibit(s) ACVR2B and ALK5 signaling in the subject.

Other aspects of the present disclosure provide a method of reducing body fat content in a subject, comprising administering to the subject an agent or a combination of agents that inhibit(s) ACVR2B and ALK5 signaling in the subject.

Yet other aspects of the present disclosure provide a method of improving glucose metabolism in a subject, comprising administering to the subject an agent or a combination of agents that inhibit(s) ACVR2B and ALK5 signaling in the subject.

In some embodiments, a single agent is administered. The single agent may inhibit, for example, ACVR2B and ALK5 signaling (e.g., may bind to both ACVR2B and ALK5). In some embodiments, the single agent may specifically inhibit ACVR2B and ALK5 signaling. In other embodiments, two (or more, e.g., three or four) agents are administered. For example, one agent may inhibit (e.g., specifically inhibit) ACVR2B signaling, while another agent may inhibit (e.g., specifically inhibit) ALK5 signaling.

In some embodiments, the agent or a combination of agents is/are administered to the subject in an effective amount to increase muscle weight in the subject by at least 40% relative to a control or baseline. For example, a single agent that inhibits (e.g., specifically inhibits) ACVR2B and ALK5 signaling (e.g., by binding to both ACVR2B and ALK5) may be administered to the subject in an effective amount to increase muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline. As another example, two agents, one that inhibits ACVR2B signaling (e.g., by binding to ACVR2B) and one that inhibits ALK5 signaling (e.g., by binding to ALK5) may be administered to the subject in an effective amount to increase muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline.

In some embodiments, the agent or combination of agents inhibit(s) ACVR2B and ALK5 signaling by binding to ACVR2B and ALK5.

In some embodiments, the agent or combination of agents inhibit(s) ACVR2B and ALK5 signaling specifically in myofibers of the subject. For example, the agent(s) may be selected from ASOs, RNAi molecules (e.g., shRNA, siRNA, or miRNA), and programmable nuclease-based gene editing molecules (e.g., CRISPR/Cas9/gRNAs, TALE/TALENs, and ZFNs) that specifically target ACVR2B and/or ALK5.

In some embodiments, the agent or combination of agents is/are administered to the subject in an effective amount to increase tricep muscle weight in the subject by at least 3%, at least 4%, or at least 5% relative to a control or baseline. For example, a single agent that inhibits (e.g., specifically inhibits) ACVR2B and ALK5 signaling (e.g., by binding to both ACVR2B and ALK5) may be administered to the subject in an effective amount to increase tricep muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, or at least 50% relative to a control or baseline. As another example, two agents, one that inhibits ACVR2B signaling (e.g., by binding to ACVR2B) and one that inhibits ALK5 signaling (e.g., by binding to ALK5) may be administered to the subject in an effective amount to increase tricep muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, or at least 50% relative to a control or baseline.

In some embodiments, the agent or combination of agents is/are administered to the subject in an effective amount to increase quadricep muscle weight in the subject by at least 3%, at least 4%, or at least 5% relative to a control or baseline. For example, a single agent that inhibits (e.g., specifically inhibits) ACVR2B and ALK5 signaling (e.g., by binding to both ACVR2B and ALK5) may be administered to the subject in an effective amount to increase quadricep muscle weight in the subject by at least 3%, at least 4%, at least 5%, at least 6%, at least 7%, at least 8%, at least 9%, at least 10%, at least 20%, at least 25%, at least 30%, at least 35%, at least 40%, at least 45%, at least 50%, at least 55%, at least 60%, at least 65%, or at least 70% relative to a control or baseline. As another example, two agents, one that inhibits ACVR2B signaling (e.g., by binding to ACVR2B) and one that inhibits ALK5 signaling (e.g., by binding to ALK5) may be administered to the subject in an effective amount to increase quadricep muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, or at least 70% relative to a control or baseline.

In some embodiments, the agent or combination of agents is/are administered to the subject in an effective amount to increase gastrocnemius/plantaris muscle weight in the subject by at least 3%, at least 4%, or at least 5% relative to a control or baseline. For example, a single agent that inhibits (e.g., specifically inhibits) ACVR2B and ALK5 signaling (e.g., by binding to both ACVR2B and ALK5) may be administered to the subject in an effective amount to increase gastrocnemius/plantaris muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, or at least 70% relative to a control or baseline. As another example, two agents, one that inhibits ACVR2B signaling (e.g., by binding to ACVR2B) and one that inhibits ALK5 signaling (e.g., by binding to ALK5) may be administered to the subject in an effective amount to increase gastrocnemius/plantaris muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, or at least 70% relative to a control or baseline.

In some embodiments, the agent or combination of agents is/are administered to the subject in an effective amount to increase pectoralis muscle weight in the subject by at least 3%, at least 4%, or at least 5% relative to a control or baseline. For example, a single agent that inhibits (e.g., specifically inhibits) ACVR2B and ALK5 signaling (e.g., by binding to both ACVR2B and ALK5) may be administered to the subject in an effective amount to increase pectoralis muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, or at least 70% relative to a control or baseline. As another example, two agents, one that inhibits (e.g., specifically inhibits) ACVR2B signaling (e.g., by binding to ACVR2B) and one that inhibits (e.g., specifically inhibits) ALK5 signaling (e.g., by binding to ALK5) may be administered to the subject in an effective amount to increase pectoralis muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, or at least 70% relative to a control or baseline.

Some aspects of the present disclosure provide a method of increasing muscle weight in a subject, comprising administering to the subject an agent or a combination of agents that inhibit(s) type I and/or type II receptor signaling in myofibers of the subject.

Other aspects of the present disclosure provide a method of reducing body fat content in a subject, comprising administering to the subject an agent or a combination of agents that inhibit(s) type I and/or type II receptor signaling in myofibers of the subject.

Yet other aspects of the present disclosure provide a method of improving glucose metabolism in a subject, comprising administering to the subject an agent or a combination of agents that inhibit(s) type I and/or type II receptor signaling in myofibers of the subject.

In some embodiments, the type I receptor is selected from the group consisting of ALK4 and ALK5. In some embodiments, the agent or combination of agents inhibit(s) ALK4. In some embodiments, the agent or combination of agents inhibit(s) ALK5. In some embodiments, the agent or combination of agents inhibit(s) ALK4 and ALK5. In some embodiments, the agent or combination of agents that inhibit(s) type I receptor signaling binds to ALK4, ALK5 or both ALK4 and ALK5.

In some embodiments, the type II receptor is selected from the group consisting of ACVR2A, ACVR2B, and TGFβRII. In some embodiments, the agent or combination of agents inhibit(s) ACVR2A. In some embodiments, the agent or combination of agents inhibit(s) ACVR2B. In some embodiments, the agent or combination of agents inhibit(s) TGFβRII. In some embodiments, the agent or combination of agents that inhibit(s) type I receptor signaling binds to ACVR2A, ACVR2B, TGFβRII, or any combination of two or three of the foregoing. In some embodiments, the agent or combination of agents inhibit(s) ALK4 and ALK5 signaling. In some embodiments, the agent or combination of agents inhibit(s) ACVR2A and ACVR2B signaling.

Some aspects of the present disclosure provide a method of increasing muscle weight in a subject, comprising administering to the subject an agent or a combination of agents that inhibit(s) (a) TGFβRII and/or (b) TGFβ1, TGFβ2, and/or TGFβ3 signaling in the subject.

Other aspects of the present disclosure provide a method of reducing body fat content in a subject, comprising administering to the subject an agent or a combination of agents that inhibit(s) (a) TGFβRII and/or (b) TGFβ1, TGFβ2, and/or TGFβ3 signaling in the subject.

Yet other aspects of the present disclosure provide a method improving glucose metabolism in a subject, comprising administering to the subject an agent or a combination of agents that inhibit(s) (a) TGFβRII and/or (b) TGFβ1, TGFβ2, and/or TGFβ3 signaling in the subject.

In some embodiments, the agent or a combination of agents is/are administered to the subject in an effective amount to increase muscle weight in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline.

In some embodiments, the agent or combination of agents inhibit(s) (a) TGFβRII and/or (b) TGFβI, II and/or III signaling by binding to (a) TGFβRII and/or (b) TGFβ1, TGFβ2, and/or TGFβ3. In some embodiments, the agent or combination of agents bind(s) to TGFβRII. In other embodiments, the agent or combination of agents bind(s) to TGFβ1. In yet other embodiments, the agent or combination of agents bind(s) to TGFβ2. In still other embodiments, the agent or combination of agents bind(s) to TGFβ3.

In some embodiments, the method further comprises administering to the subject an agent or a combination of agents that inhibit(s) type I receptor signaling in the subject. In some embodiments, the method further comprises administering to the subject an agent or a combination of agents that inhibit(s) type II receptor signaling in the subject. In some embodiments, the method further comprises administering to the subject an agent or a combination of agents that inhibit(s) type I receptor signaling and activin A type II receptor signaling in the subject.

In some embodiments, the type I receptor is selected from the group consisting of ALK4 and ALK5. In some embodiments, the type I receptor is ALK4. In some embodiments, the type I receptor is ALK5.

In some embodiments, the type II receptor is selected from the group consisting of ACVR2A, ACVR2B, and TGFβRII. In some embodiments, the type II receptor is ACVR2A. In some embodiments, the type II receptor is ACVR2B. In some embodiments, the type II receptor is TGFβRII.

In some embodiments, the method comprises administering to the subject an agent or a combination of agents that inhibit(s) signaling through TGFβRII and one or more of the following pairs of receptors: (a) ALK4 and ALK5; (b) ACVR2A and AVCR2B; (c) ALK4 and ACVR2A; (d) ALK4 and ACVR2B; (e) ALK5 and ACVR2A; and (f) ALK5 and ACRV2B. In some embodiments, the method comprises administering to the subject an agent or a combination of agents that inhibit(s) signaling through TGFβRII, ALK4 and ALK5. In some embodiments, the method comprises administering to the subject an agent or a combination of agents that inhibit(s) signaling through TGFβRII, ACVR2A and AVCR2B. In some embodiments, the method comprises administering to the subject an agent or a combination of agents that inhibit(s) signaling through TGFβRII, ALK4 and ACVR2A. In some embodiments, the method comprises administering to the subject an agent or a combination of agents that inhibit(s) signaling through TGFβRII, ALK4 and ACVR2B. In some embodiments, the method comprises administering to the subject an agent or a combination of agents that inhibit(s) signaling through TGFβRII, ALK5 and ACVR2A. In some embodiments, the method comprises administering to the subject an agent or a combination of agents that inhibit(s) signaling through TGFβRII, ALK5 and ACRV2B.

Some aspects of the present disclosure provide a method of increasing bone mineral density, bone volume, and/or bone density in a subject, comprising administering to the subject an agent or a combination of agents that inhibit(s) ALK4 and/or ALK5 signaling in the subject. In some embodiments, bone mineral density, bone volume, and/or bone density is increased in the hip, lumbar spine, forearm or whole body of the subject.

In some embodiments, a single agent is administered. The single agent may inhibit, for example, ALK4 and ALK5 signaling (e.g., may bind to both ALK4 and ALK5). In some embodiments, the single agent may specifically inhibit ALK4 and ALK5 signaling. In other embodiments, two (or more, e.g., three or four) agents are administered. For example, one agent may inhibit (e.g., specifically inhibit) ALK4 signaling, while another agent may inhibit ALK5 signaling (e.g., specifically inhibit).

Non-limiting examples of such agents include, antibodies, soluble receptors, small molecules, and other non-peptide molecules, such as antisense oligonucleotides (ASOs), RNA interference (RNAi) molecules, and programmable-nuclease-based gene editing systems.

In some embodiments, the agent or combination of agents is/are administered to the subject in an effective amount to (directly or indirectly) increase bone mineral density, bone volume, and/or bone density in the subject by at least 40% relative to a control or baseline. For example, a single agent that inhibits (e.g., specifically inhibits) ALK4 and/or ALK5 signaling (e.g., by binding to ALK4, ALK5, or both ALK4 and ALK5) may be administered to the subject in an effective amount to increase bone mineral density, bone volume, and/or bone density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline. As another example, two agents, one that inhibits ALK4 signaling (e.g., by binding to ALK4) and one that inhibits ALK5 signaling (e.g., by binding to ALK5) may be administered to the subject in an effective amount to increase bone mineral density, bone volume, and/or bone density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline. A control may be administration of a placebo (e.g., saline) or baseline (e.g., bone mineral density within 24 hours prior to administration of the one or more agent(s))).

In some embodiments, the agent or combination of agents inhibit(s) ALK4 and/or ALK5 signaling by binding to (e.g., specifically binding to) ALK4 and/or ALK5.

In some embodiments, the agent or combination of agents inhibit(s) ALK4 and/or ALK5 signaling specifically in osteoblasts of the subject. For example, the agent(s) may be selected from ASOs, RNAi molecules (e.g., shRNA, siRNA, or miRNA), and programmable nuclease-based gene editing molecules (e.g., CRISPR/Cas9/gRNAs, TALE/TALENs, and ZFNs) that specifically target ALK4 and/or ALK5.

In some embodiments, the agent or a combination of agents is/are administered to the subject in an effective amount to increase total body bone mineral density by at least 3%, at least 4%, or at least 5% relative to a control or baseline.

In some embodiments, the agent or a combination of agents is/are administered to the subject in an effective amount to increase bone mineral density by at least 3%, at least 4%, or at least 5% at a site selected from the group consisting of lumbar spine, radius, ulna, and pelvis, relative to a control or baseline.

In some embodiments, the agent or combination of agents is/are administered to the subject in an effective amount to increase lumbar spine bone mineral density in the subject by at least 50% relative to a control or baseline. For example, a single agent that inhibits (e.g., specifically inhibits) ALK4 and/or ALK5 signaling (e.g., by binding to ALK4, ALK5, or both ALK4 and ALK5) may be administered to the subject in an effective amount to increase lumbar spine bone mineral density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline. As another example, two agents, one that inhibits ALK4 signaling (e.g., by binding to ALK4) and one that inhibits ALK5 signaling (e.g., by binding to ALK5) may be administered to the subject in an effective amount to increase lumbar spine bone mineral density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline.

In some embodiments, the agent or combination of agents is/are administered to the subject in an effective amount to increase radius and/or ulna bone mineral density in the subject by at least 3% relative to a control or baseline. For example, a single agent that inhibits (e.g., specifically inhibits) ALK4 and/or ALK5 signaling (e.g., by binding to ALK4, ALK5, or both ALK4 and ALK5) may be administered to the subject in an effective amount to increase radius and/or ulna bone mineral density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline. As another example, two agents, one that inhibits ALK4 signaling (e.g., by binding to ALK4) and one that inhibits ALK5 signaling (e.g., by binding to ALK5) may be administered to the subject in an effective amount to increase radius and/or ulna bone mineral density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline.

In some embodiments, the agent or combination of agents is/are administered to the subject in an effective amount to increase pelvis bone mineral density in the subject by at least 3%, at least 4%, or at least 5% relative to a control or baseline. For example, a single agent that inhibits (e.g., specifically inhibits) ALK4 and/or ALK5 signaling (e.g., by binding to ALK4, ALK5, or both ALK4 and ALK5) may be administered to the subject in an effective amount to increase pelvis bone mineral density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline. As another example, two agents, one that inhibits ALK4 signaling (e.g., by binding to ALK4) and one that inhibits ALK5 signaling (e.g., by binding to ALK5) may be administered to the subject in an effective amount to increase pelvis bone mineral density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline.

Some aspects of the present disclosure provide a method of increasing bone mineral density, bone volume, and/or bone density in a subject, comprising administering to the subject an agent or a combination of agents that inhibit(s) ACVR2A and ALK5 signaling in the subject.

In some embodiments, a single agent is administered. The single agent may inhibit, for example, ACVR2A and ALK5 signaling (e.g., may bind to both ACVR2A and ALK5). In some embodiments, the single agent may specifically inhibit ACVR2A and ALK5 signaling. In other embodiments, two (or more, e.g., three or four) agents are administered. For example, one agent may inhibit (e.g., specifically inhibit) ACVR2A signaling, while another agent may inhibit (e.g., specifically inhibit) ALK5 signaling.

In some embodiments, the agent or a combination of agents is/are administered to the subject in an effective amount to increase bone mineral density, bone volume, and/or bone density in the subject by at least 3%, at least 4%, or at least 5% relative to a control or baseline. For example, a single agent that inhibits (e.g., specifically inhibits) ACVR2A and ALK5 signaling (e.g., by binding to both ACVR2A and ALK5) may be administered to the subject in an effective amount to increase bone mineral density, bone volume, and/or bone density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline. As another example, two agents, one that inhibits ACVR2A signaling (e.g., by binding to ACVR2A) and one that inhibits ALK5 signaling (e.g., by binding to ALK5) may be administered to the subject in an effective amount to increase bone mineral density, bone volume, and/or bone density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline.

In some embodiments, the agent or combination of agents inhibit(s) ACVR2A and ALK5 signaling by binding to ACVR2A and ALK5.

In some embodiments, the agent or combination of agents inhibit(s) ACVR2A and ALK5 signaling specifically in osteoblasts of the subject. For example, the agent(s) may be selected from ASOs, RNAi molecules (e.g., shRNA, siRNA, or miRNA), and programmable nuclease-based gene editing molecules (e.g., CRISPR/Cas9/gRNAs, TALE/TALENs, and ZFNs) that specifically target ACVR2A and/or ALK5.

In some embodiments, the agent or a combination of agents is/are administered to the subject in an effective amount to increase total body bone mineral density by at least 3%, at least 4%, or at least 5% relative to a control or baseline.

In some embodiments, the agent or a combination of agents is/are administered to the subject in an effective amount to increase bone mineral density by at least 3%, at least 4%, or at least 5% at a site selected from the group consisting of lumbar spine, radius, ulna, and pelvis, relative to a control or baseline.

In some embodiments, the agent or combination of agents is/are administered to the subject in an effective amount to increase lumbar spine bone mineral density in the subject by at least 3%, at least 4%, or at least 5% relative to a control or baseline. For example, a single agent that inhibits (e.g., specifically inhibits) ACVR2A and ALK5 signaling (e.g., by binding to both ACVR2A and ALK5) may be administered to the subject in an effective amount to increase lumbar spine bone mineral density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, or at least 50% relative to a control or baseline. As another example, two agents, one that inhibits ACVR2A signaling (e.g., by binding to ACVR2A) and one that inhibits ALK5 signaling (e.g., by binding to ALK5) may be administered to the subject in an effective amount to increase lumbar spine bone mineral density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, or at least 50% relative to a control or baseline.

In some embodiments, the agent or combination of agents is/are administered to the subject in an effective amount to increase radius and/or ulna bone mineral density in the subject by at least 3%, at least 4%, or at least 5% relative to a control or baseline. For example, a single agent that inhibits (e.g., specifically inhibits) ACVR2A and ALK5 signaling (e.g., by binding to both ACVR2A and ALK5) may be administered to the subject in an effective amount to increase radius and/or ulna bone mineral density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, or at least 70% relative to a control or baseline. As another example, two agents, one that inhibits ACVR2A signaling (e.g., by binding to ACVR2A) and one that inhibits ALK5 signaling (e.g., by binding to ALK5) may be administered to the subject in an effective amount to increase radius and/or ulna bone mineral density in the subject by at least 3%, at least 4%, at least 5%, at least 6%, at least 7%, at least 8%, at least 9%, at least 10%, at least 15%, at least 20%, at least 25% at least 30%, at least 35%, at least 40%, at least 45%, at least 50%, at least 55%, at least 60%, at least 65%, or at least 70% relative to a control or baseline.

In some embodiments, the agent or combination of agents is/are administered to the subject in an effective amount to increase pelvis bone mineral density in the subject by at least 3%, at least 4%, or at least 5% relative to a control or baseline. For example, a single agent that inhibits (e.g., specifically inhibits) ACVR2A and ALK5 signaling (e.g., by binding to both ACVR2A and ALK5) may be administered to the subject in an effective amount to increase pelvis bone mineral density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, or at least 80% relative to a control or baseline. As another example, two agents, one that inhibits ACVR2A signaling (e.g., by binding to ACVR2A) and one that inhibits ALK5 signaling (e.g., by binding to ALK5) may be administered to the subject in an effective amount to increase pelvis bone mineral density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, or at least 80% relative to a control or baseline.

In some embodiments, a single agent is administered. The single agent may inhibit, for example, ACVR2B and ALK5 signaling (e.g., may bind to both ACVR2B and ALK5). In some embodiments, the single agent may specifically inhibit ACVR2B and ALK5 signaling. In other embodiments, two (or more, e.g., three or four) agents are administered. For example, one agent may inhibit (e.g., specifically inhibit) ACVR2B signaling, while another agent may inhibit (e.g., specifically inhibit) ALK5 signaling.

In some embodiments, the agent or a combination of agents is/are administered to the subject in an effective amount to increase bone mineral density, bone volume, and/or bone density in the subject by at least 40% relative to a control or baseline. For example, a single agent that inhibits (e.g., specifically inhibits) ACVR2B and ALK5 signaling (e.g., by binding to both ACVR2B and ALK5) may be administered to the subject in an effective amount to increase bone mineral density, bone volume, and/or bone density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline. As another example, two agents, one that inhibits ACVR2B signaling (e.g., by binding to ACVR2B) and one that inhibits ALK5 signaling (e.g., by binding to ALK5) may be administered to the subject in an effective amount to increase bone mineral density, bone volume, and/or bone density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline.

In some embodiments, the agent or combination of agents inhibit(s) ACVR2B and ALK5 signaling by binding to ACVR2B and ALK5.

In some embodiments, the agent or combination of agents inhibit(s) ACVR2B and ALK5 signaling specifically in osteoblasts of the subject. For example, the agent(s) may be selected from ASOs, RNAi molecules (e.g., shRNA, siRNA, or miRNA), and programmable nuclease-based gene editing molecules (e.g., CRISPR/Cas9/gRNAs, TALE/TALENs, and ZFNs) that specifically target ACVR2B and/or ALK5.

In some embodiments, the agent or a combination of agents is/are administered to the subject in an effective amount to increase total body bone mineral density by at least 3%, at least 4%, or at least 5% relative to a control or baseline.

In some embodiments, the agent or a combination of agents is/are administered to the subject in an effective amount to increase bone mineral density by at least 3%, at least 4%, or at least 5% at a site selected from the group consisting of lumbar spine, radius, ulna, and pelvis, relative to a control or baseline.

In some embodiments, the agent or combination of agents is/are administered to the subject in an effective amount to increase lumbar spine bone mineral density in the subject by at least 3%, at least 4%, or at least 5% relative to a control or baseline. For example, a single agent that inhibits (e.g., specifically inhibits) ACVR2B and ALK5 signaling (e.g., by binding to both ACVR2B and ALK5) may be administered to the subject in an effective amount to increase lumbar spine bone mineral density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, or at least 50% relative to a control or baseline. As another example, two agents, one that inhibits ACVR2B signaling (e.g., by binding to ACVR2B) and one that inhibits ALK5 signaling (e.g., by binding to ALK5) may be administered to the subject in an effective amount to increase lumbar spine bone mineral density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, or at least 50% relative to a control or baseline.

In some embodiments, the agent or combination of agents is/are administered to the subject in an effective amount to increase radius and/or ulna bone mineral density in the subject by at least 3%, at least 4%, or at least 5% relative to a control or baseline. For example, a single agent that inhibits (e.g., specifically inhibits) ACVR2B and ALK5 signaling (e.g., by binding to both ACVR2B and ALK5) may be administered to the subject in an effective amount to increase radius and/or ulna bone mineral density in the subject by at least 3%, at least 4%, at least 5%, at least 6%, at least 7%, at least 8%, at least 9%, at least 10%, at least 20%, at least 25%, at least 30%, at least 35%, at least 40%, at least 45%, at least 50%, at least 55%, at least 60%, at least 65%, or at least 70% relative to a control or baseline. As another example, two agents, one that inhibits ACVR2B signaling (e.g., by binding to ACVR2B) and one that inhibits ALK5 signaling (e.g., by binding to ALK5) may be administered to the subject in an effective amount to increase radius and/or ulna bone mineral density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, or at least 70% relative to a control or baseline.

In some embodiments, the agent or combination of agents is/are administered to the subject in an effective amount to increase pelvis bone mineral density in the subject by at least 3%, at least 4%, or at least 5% relative to a control or baseline. For example, a single agent that inhibits (e.g., specifically inhibits) ACVR2B and ALK5 signaling (e.g., by binding to both ACVR2B and ALK5) may be administered to the subject in an effective amount to increase pelvis bone mineral density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, or at least 70% relative to a control or baseline. As another example, two agents, one that inhibits ACVR2B signaling (e.g., by binding to ACVR2B) and one that inhibits ALK5 signaling (e.g., by binding to ALK5) may be administered to the subject in an effective amount to increase pelvis bone mineral density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, or at least 70% relative to a control or baseline.

In some embodiments, the agent or combination of agents is/are administered to the subject in an effective amount to increase vertebrae bone mineral density in the subject by at least 3%, at least 4%, or at least 5% relative to a control or baseline. For example, a single agent that inhibits (e.g., specifically inhibits) ACVR2B and ALK5 signaling (e.g., by binding to both ACVR2B and ALK5) may be administered to the subject in an effective amount to increase vertebrae bone mineral density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, or at least 70% relative to a control or baseline. As another example, two agents, one that inhibits (e.g., specifically inhibits) ACVR2B signaling (e.g., by binding to ACVR2B) and one that inhibits (e.g., specifically inhibits) ALK5 signaling (e.g., by binding to ALK5) may be administered to the subject in an effective amount to increase vertebrae bone mineral density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, or at least 70% relative to a control or baseline.

Some aspects of the present disclosure provide a method of increasing bone mineral density, bone volume, and/or bone density in a subject, comprising administering to the subject an agent or a combination of agents that inhibit(s) type 1 and/or type 2 receptor signaling in osteoblasts of the subject.

In some embodiments, the type 1 receptor is selected from the group consisting of ALK4 and ALK5. In some embodiments, the agent or combination of agents inhibit(s) ALK4. In some embodiments, the agent or combination of agents inhibit(s) ALK5. In some embodiments, the agent or combination of agents inhibit(s) ALK4 and ALK5. In some embodiments, the agent or combination of agents that inhibit(s) type 1 receptor signaling binds to ALK4, ALK5 or both ALK4 and ALK5.

In some embodiments, the type 2 receptor is selected from the group consisting of ACVR2A, ACVR2B, and TGFβRII. In some embodiments, the agent or combination of agents inhibit(s) ACVR2A. In some embodiments, the agent or combination of agents inhibit(s) ACVR2B. In some embodiments, the agent or combination of agents inhibit(s) TGFβRII. In some embodiments, the agent or combination of agents that inhibit(s) type 1 receptor signaling binds to ACVR2A, ACVR2B, TGFβRII, or any combination of two or three of the foregoing. In some embodiments, the agent or combination of agents inhibit(s) ALK4 and ALK5 signaling. In some embodiments, the agent or combination of agents inhibit(s) ACVR2A and ACVR2B signaling.

Some aspects of the present disclosure provide a method of increasing muscle weight in a subject, comprising administering to the subject an agent or a combination of agents that inhibit(s) (a) TGFβRII and/or (b) TGFβ1, TGFβ2, and/or TGFβ3 signaling in the subject.

Other aspects of the present disclosure provide a method of reducing body fat content in a subject, comprising administering to the subject an agent or a combination of agents that inhibit(s) (a) TGFβRII and/or (b) TGFβ1, TGFβ2, and/or TGFβ3 signaling in the subject.

Yet other aspects of the present disclosure provide a method improving glucose metabolism in a subject, comprising administering to the subject an agent or a combination of agents that inhibit(s) (a) TGFβRII and/or (b) TGFβ1, TGFβ2, and/or TGFβ3 signaling in the subject.

In some embodiments, the agent or a combination of agents is/are administered to the subject in an effective amount to increase total body bone mineral density by at least 3%, at least 4%, or at least 5% relative to a control or baseline.

In some embodiments, the agent or a combination of agents is/are administered to the subject in an effective amount to increase bone mineral density by at least 3%, at least 4%, or at least 5% at a site selected from the group consisting of lumbar spine, radius, ulna, and pelvis, relative to a control or baseline.

In some embodiments, the agent or a combination of agents is/are administered to the subject in an effective amount to increase bone mineral density, bone volume, and/or bone density in the subject by at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% relative to a control or baseline.

In some embodiments, the agent or combination of agents inhibit(s) (a) TGFβRII and/or (b) TGFβI, II and/or III signaling by binding to (a) TGFβRII and/or (b) TGFβ1, TGFβ2, and/or TGFβ3. In some embodiments, the agent or combination of agents bind(s) to TGFβRII. In other embodiments, the agent or combination of agents bind(s) to TGFβ1. In yet other embodiments, the agent or combination of agents bind(s) to TGFβ2. In still other embodiments, the agent or combination of agents bind(s) to TGFβ3.

In some embodiments, the method further comprises administering to the subject an agent or a combination of agents that inhibit(s) type 1 receptor signaling in the subject. In some embodiments, the method further comprises administering to the subject an agent or a combination of agents that inhibit(s) type 2 receptor signaling in the subject. In some embodiments, the method further comprises administering to the subject an agent or a combination of agents that inhibit(s) type 1 receptor signaling and activin A type 2 receptor signaling in the subject.

In some embodiments, the type 1 receptor is selected from the group consisting of ALK4 and ALK5. In some embodiments, the type 1 receptor is ALK4. In some embodiments, the type 1 receptor is ALK5.

In some embodiments, the type 2 receptor is selected from the group consisting of ACVR2A, ACVR2B, and TGFβRII. In some embodiments, the type 2 receptor is ACVR2A. In some embodiments, the type 2 receptor is ACVR2B. In some embodiments, the type 2 receptor is TGFβRII.

In some embodiments, the method comprises administering to the subject an agent or a combination of agents that inhibit(s) signaling through TGFβRII and one or more of the following pairs of receptors: (a) ALK4 and ALK5; (b) ACVR2A and AVCR2B; (c) ALK4 and ACVR2A; (d) ALK4 and ACVR2B; (e) ALK5 and ACVR2A; and (f) ALK5 and ACRV2B. In some embodiments, the method comprises administering to the subject an agent or a combination of agents that inhibit(s) signaling through TGFβRII, ALK4 and ALK5. In some embodiments, the method comprises administering to the subject an agent or a combination of agents that inhibit(s) signaling through TGFβRII, ACVR2A and AVCR2B. In some embodiments, the method comprises administering to the subject an agent or a combination of agents that inhibit(s) signaling through TGFβRII, ALK4 and ACVR2A. In some embodiments, the method comprises administering to the subject an agent or a combination of agents that inhibit(s) signaling through TGFβRII, ALK4 and ACVR2B. In some embodiments, the method comprises administering to the subject an agent or a combination of agents that inhibit(s) signaling through TGFβRII, ALK5 and ACVR2A. In some embodiments, the method comprises administering to the subject an agent or a combination of agents that inhibit(s) signaling through TGFβRII, ALK5 and ACRV2B.

### BRIEF DESCRIPTION OF THE DRAWINGS

FIGS. 1A-1F. Effect of targeting type II and type I receptors in myofibers on muscle weights. (FIG. 1A-1B) Relative weights of pectoralis, triceps, quadriceps, and gastrocnemius/plantaris muscles in mice in which Acvr2 and/or Acvr2b (FIG. 1A) or Alk4 and/or Alk5 (FIG. 1B) were targeted. Numbers are expressed as percent increase/decrease relative to the same receptor genotypes but in the absence of Myl1-Cre. (FIG. 1C) Gastrocnemius/plantaris muscle weights of individual wild type C57BL/6 and Mstn −/− mice or individual mice in which Acvr2/Acvr2b or Alk4/Alk5 were targeted in myofibers. Bars indicates mean values. (FIG. 1D) Relative muscle weights of mice in which an individual type II receptor (Acvr2 or Acvr2b) was targeted along with an individual type I receptor (Alk4 or Alk5). (FIGS. 1E-1F) Relative muscle weights of mice in which an individual type II or type I receptor was targeted along with Cfc1b (FIG. 1E) or Mstn (FIG. 1F). Numbers are expressed as percent increase/decrease relative to the same receptor genotypes but in the absence of Myl1-Cre, and the order of bars is the same as in FIGS. 1A-1B. The numbers shown in FIGS. 1A-1B and FIGS. 1D-1F were calculations based on muscle weights shown in Tables 1-3, which also contain the numbers of mice in each group. ^{a}p<0.001 vs. cre−; ^{b}p<0.01 vs. cre−; ^{c}p<0.05 vs. cre−; ^{d}p<0.001 vs. Mstn fl/fl, cre+; ^{e}p<0.01 vs. vs. Mstn fl/fl, cre+; ^{f}p<0.05 vs. vs. Mstn fl/fl, cre+.

FIGS. 2A-2C. Lack of effect of targeting Acvr2 and Acvr2b in myofibers on muscle regeneration following chemical injury. (FIG. 2A) Distribution of myofiber cross-sectional areas (CSA), mean CSA (FIG. 2B), and number of Pax7+ cells (FIG. 2C) in Acvr2 fl/fl, Acvr2b fl/fl mice with or without Myl1-cre either uninjured or 5 or 21 days post-injury.

FIG. 3. Total body fat content by DXA analysis, plasma leptin levels, fasting blood glucose levels, and fasting plasma insulin levels in one-year-old mice lacking MSTN and mice in which both type II receptors were targeted in myofibers. Numbers of mice in each group are shown underneath the bars.

FIGS. 4A-4C. Effect of a high fat diet on Mstn −/− mice and mice in which Acvr2 and Acvr2b have been targeted in myofibers. (FIG. 4A) Weight gain in male mice placed on a high fat diet starting at 12 weeks of age. (FIG. 4B) Fasting blood glucose levels and (FIG. 4C) glucose tolerance tests in 12-week-old male mice on standard diets or after placement on a high fat diet for 4 weeks. In (FIG. 4C), the numbers of mice in each group are the same as shown in panel (FIG. 4A).

FIGS. 5A-5C. Lack of bone effects of targeting Acvr2 and Acvr2b in myofibers. (FIG. 5A) Top panel, DXA analysis of wild type C57BL/6 mice either uninjected (n=9) or injected weekly with the ACVR2B/Fc decoy receptor at a dose of 10 mg/kg (n=8) starting at 10 weeks of age. Bottom panel, DXA analysis of Acvr2 flox/flox, Acvr2b flox/flox mice with (n=8) and without (n=12) the Myl1-cre transgene at the same ages as in the top panel. *p<0.05, **p<0.001. (FIG. 5B) MicroCT images of femurs taken from these same mice at 16 weeks of age. (FIG. 5C) Bone volume/total volume fraction, trabecular thickness, trabecular number), apparent density, and cortical thickness of femurs and L4 and L5 vertebrae determined by microCT analysis in these mice at 16 weeks of age. Numbers of mice in each group are shown underneath the bars.

FIGS. 6A-6F. Effect of Fst mutant alleles on skeletal muscle. (FIG. 6A) Weights of gastrocnemius muscles versus Fst RNA expression levels in mice carrying various combinations of Fst mutant alleles. Numbers are normalized to values for Fst muscles. RNA expression levels were measured by qPCR in 3 mice per group. (FIG. 6B) Plasma FST levels in mice carrying various Fst mutant alleles with or without Myl1-cre or Cdx2-cre transgenes (n=4 per group). (FIG. 6C) Relative weights of pectoralis (red), triceps (gray), quadriceps (blue), and gastrocnemius/plantaris (green) muscles in mice carrying various Fst mutant alleles with or without Myl1-cre. Numbers are expressed as percent increase/decrease relative to Fst^{+/+} mice and were calculated from the data shown in Table 1. (FIG. 6D) Relative weights of muscles of Fst^{fl/-} mice in the absence (blue bars) or presence (orange bars) of Cdx2-cre. Numbers are expressed as percent increase/decrease relative to Fst^{+/+} mice. P, pectoralis; T, triceps; Q, quadriceps; G, gastrocnemius. ^{a}p<0.001, ^{b}p<0.05 versus Cdx2-cre negative mice. (FIG. 6E) Distribution of fiber sizes in gastrocnemius muscles of F66 and Fst^{+/+} mice (upper panel) or Fst^{fl/-}; Cdx2-cre negative and positive mice (lower panel). Fiber diameters were measured in muscles isolated from 3 mice (250 fibers per mouse) and pooled for plotting. (FIG. 6F) Plasma MSTN levels in Fst^{fl/-}; Cdx2-cre negative and positive mice (n=8 per group). M, males; F, females.

FIGS. 7A-7B. Fiber type shifts in Fst mutant mice. (FIG. 7A) Percent type I, type 2a, and type 2b fibers in gastrocnemius and triceps muscles of Fst^{fl/-}; Cdx2-cre and F66 mice (n=3 per group). (FIG. 7B) Relative RNA expression levels for genes encoding sarcomeric protein isoforms and kinases. Numbers were determined by RNA-seq analysis and are presented as the loge ratio of values obtained for Cdx2-cre positive to Cdx2-cre negative (n=9 per group). Blue, slow isoforms; orange, fast isoforms; green, developmental and cardiac isoforms. ^{a}p<0.001, ^{b}p<0.01, ^{c}p<0.05

FIGS. 7A-7C. Lipid accumulation in Fst mutant mice. (FIG. 7A) Oil Red O stains of gastrocnemius sections of Fst^{fl/-}; Cdx2-cre negative and positive mice. (FIG. 7B) Fluorescence intensity of Nile red-stained sections of gastrocnemius and triceps muscles of Fst^{fl/-}; Cdx2-cre and F66 mice. Numbers represent means of measurements taken from Fst^{fl/-}; Cdx2-cre negative, Fst^{fl/-}; Cdx2-cre positive, wild-type, and F66 mice, with n=5, 5, 7, and 7 mice, respectively, and with 4 representative sections analyzed for each mouse. (FIG. 7C) Up-regulated pathways identified by RNA-seq analysis of gastrocnemius muscles isolated from Fst^{fl/-}; Cdx2-cre positive compared to cre negative mice (n=9 per group).

FIGS. 8A-8C. Effect of Fst mutant alleles on bone. Micro-CT analysis of (FIG. 8A) femurs and (FIG. 8B) L4 and L5 vertebrae of Fst^{flox/flox}, Fst^{+/+}, and Fst^{+/-} mice. ^{a}p<0.001, ^{b}p<0.01, ^{c}p<0.05. (FIG. 8C) Micro-CT analysis of femurs and humeri isolated from Fst^{fl/-}; Cdx2-cre negative and positive mice. F, females; M, males. In FIGS. 4A-4C, numbers of mice per group are shown at bottom.

FIGS. 9A-9B. Effect of targeting type 2 and type 1 receptors in osteoblasts. (FIG. 9A) Micro-CT analysis of humeri, femurs, and L5 vertebrae of Acvr2^{flox/flox}; Acvr2b^{flox/flox}; Oc-cre negative (n=9 females, 9 males), Acvr2^{flox/flox}; Acvr2b^{flox/flox}; Oc-cre positive (n=9 females, 6 males), Alk4^{flox/flox}; Alk5^{flox/flox}; Oc-cre negative (n =6 females, 9 males), and Alk4^{flox/flox}; Alk5^{flox/flox}; Oc-cre positive (n=6 females, 7 males) mice. ^{a}p<0.001, ^{b}p<0.01, ^{c}p<0.05. (FIG. 9B) Representative micro-CT images.

FIGS. 10A-10C. Effect of targeting Mstn and Inhba in the posterior half of mice. (FIG. 10A) Lean body mass (upper panel) and bone mineral density (lower panel) of Mstn^{flox/flox}; Inhba^{flox/flox}; Cdx2-cre negative (n=8) and positive (n=8) mice by DXA analysis. Data are shown for whole body or just the anterior or posterior half of the body. (FIG. 10B) Muscle weight increases in Mstn^{flpx/flox}; Inhba^{flox/flox}; Cdx2-cre mice relative to cre negative control mice. P, pectoralis; T, triceps; Q, quadriceps; G, gastrocnemius. (FIG. 10C) Micro-CT analysis of humeri, femurs, and L5 vertebrae of Mstn^{flox/flox}; Inhba^{flox/flox}; Cdx2-cre. ^{a}p<0.001, ^{b}p<0.01, ^{c}p<0.05.

FIG. 11 shows Venn diagrams of the numbers of genes whose RNA expression levels are either up- or down-regulated in gastrocnemius muscles isolated from either Fst^{flox/-}; Cdx2-cre (relative to cre negative mice) or F66 mice (relative to wild-type mice).

FIG. 12 shows expression levels of Mstn RNA in various muscles isolated from Fst^{flox/-} mice either positive or negative for Cdx2-cre. Expression levels in cre negative mice were arbitrarily set to one for each muscle group.

### DETAILED DESCRIPTION

Myostatin is a secreted protein that is made by skeletal muscle, circulates in the blood, and acts to limit muscle growth. Signaling of myostatin and other activin-like ligands through TGF-β superfamily receptors, such as the type 1 receptors ALK4 and ALK5 and the type 2 receptors ACVR2A and ACVR2B, regulates numerous developmental pathways, including muscle and bone growth. As a result, the myostatin signaling pathway has been the focus of extensive drug development efforts for indications characterized by muscle loss. These indications include muscle degenerative diseases (muscular dystrophy), muscle wasting caused by chronic diseases (e.g., cancer, AIDS, sepsis, COPD, kidney disease, heart disease), and muscle loss due to disuse (e.g., elderly,bedridden, and wheelchair-using individuals). Several biotechnology and pharmaceutical companies have developed myostatin inhibitors that have been tested in clinical trials, many of which reached phase 2 or 3 clinical trials. Although these trials have documented increases in muscle mass as a result of treatment, the effects have been small, and no one has yet documented any clinically meaningful benefits from treatment. None of these myostatin inhibitors have reached drug approval.

In parallel with its effects on muscle growth, myostatin also limits bone density by inhibiting osteoblast differentiation and promoting osteoclast activity. Consequently, this signaling pathway is also the focus of research and development for treating indications characterized by bone loss, such as osteoporosis, Cushing's syndrome, pituitary disorders, and hyperthyroidism, as well as bone loss due to inactivity or chronic diseases described above. Current therapies for treating bone loss include calcium supplementation to promote new bone deposition, and alendronic acid (Fosamax) to inhibit bone resorption by osteoclasts. However, the effectiveness of these therapies is limited, with minimal success in replenishing lost bone.

Studies in Mstn −/− mice have shown that the function of MSTN as a negative regulator of muscle mass and bone mineral density is partially redundant with activin A, a TGF-β family member. MSTN and activin A share many key regulatory and signaling components. For example, the activities of both MSTN and activin A can be modulated extracellularly by naturally occurring inhibitory binding proteins, including follistatin and the follistatin-related protein, FSTL-3 or FLRG. Furthermore, mice that express greater amounts of follistatin, which sequesters myostatin and prevents it from acting as a receptor agonist, experience increased bone density than wild-type mice or mice in which the FST gene is not expressed in the bone microenvironment. Moreover, MSTN and activin A also appear to share receptor components. Based on in vitro studies, MSTN binds initially to the type 2 receptors, ACVR2 and ACVR2B (also called ActRIIA and ActRIIB) followed by engagement of the type 1 receptors, ALK4 and ALK5. Genetic evidence supports a role for both ACVR2 and ACVR2B in mediating MSTN signaling, regulating muscle mass, and regulating bone mineral density in vivo. Specifically, mice expressing a truncated, dominant negative form of ACVR2B in skeletal muscle or carrying deletion mutations in Acvr2 and/or Acvr2b have significantly increased muscle mass. Lack of Acvr2 and/or Acvr2b is similarly believed to inhibit the ability of myostatin to signal in bone. The consequence of complete loss of both receptors is unknown, however, because double homozygous mutants die early during embryogenesis. Moreover, the roles that the two type 1 receptors, ALK4 and ALK5, play in regulating MSTN and activin A signaling in muscle mass and bone density in vivo have not yet been documented using genetic approaches. Herein, floxed alleles were used for each of the type 2 and type 1 receptor genes to target these receptors alone and in combination in muscle fibers (myofibers) or bone-producing cells (osteoblasts). The data provided herein demonstrates that these receptors are functionally redundant and that signaling through each of these receptors contributes to the overall control of muscle mass and bone mineral density. It now seems clear that drugs capable of targeting both myostatin and activin A will have a much greater effect in humans in terms of increasing muscle mass and bone mineral density, thereby counteracting the progression of muscle loss and bone degeneration.

Thus, the present disclosure provides, in some aspects, methods and compositions that utilize inhibitors that have a broader range of specificity (broader than just myostatin) while avoiding undesired effects in other tissues as a result of blocking the other signaling proteins. Provided herein is an extensive analysis of the receptors that are used in skeletal muscle cells and osteoblasts by myostatin and activin A for signaling. These ligands utilize a two-component system for signaling, with initial binding to a type 2 receptor (ACVR2A or ACVR2B) and subsequent engagement of a type 1 receptor (ALK4 or ALK5). The data herein show, for the first time, that there is functional redundancy in this system such that all four possible combinations of type 2 and type 1 receptors are utilized, suggesting that in order to generate the maximal effect, one might target all of these combinations. Unexpectedly, however, additional data herein suggest that targeting ALK5 and ACVR2A in combination produces the greatest effect of the pairwise combinations of type 2 and type 1 receptors, raising the possibility that a drug (or combination of drugs) capable of blocking specifically this combination of receptors might produce a significant effect in human patients. These findings are also significant because the ability of these two components to work together has not been described in other tissues.

Thus, provided herein, in some aspects, are therapeutic agents and the use of therapeutic agents that inhibit (e.g., specifically inhibit) signaling through ALK5 and ACVR2. Non-limiting examples of such agents, described in further detail below, include an ACVR2/ALK5 hybrid decoy receptor (e.g., a heterodimer of the extracellular domains of each of these receptors fused to a scaffold, such as an immunoglobulin Fc domain) and a peptide or small molecule inhibitors capable of disrupting the ACVR2-ALK5 interaction.

### Myostatin Signaling Pathways

The present disclosure provides, in some aspects, methods of administering inhibitors of type 1 receptor and/or type 2 receptor signaling to a subject, thereby reducing or blocking the activity of myostatin. Myostatin is a protein that is produced by muscle cells and associates with type 1 receptors and/or type 2 receptors on cell surfaces.

The first step in myostatin signaling is the association of myostatin with a type 2 receptor, such as ACVR2A, ACVR2B, or TGFβRII. Following association with myostatin, the type 2 receptor then associates with a type 1 receptor, forming a type 1 receptor/type 2 receptor complex. This complex then activates transcription factors, such as SMAD2 and SMAD3, which modulate gene expression in the cell and ultimately inhibit growth. In immature muscle cells (myoblasts), these signals inhibit differentiation into mature muscle fibers. Myostatin signaling also inhibits the activity of the protein kinase B (PKB), also known as Akt, a serine/threonine protein kinase that plays a key role in multiple cellular processes, including glucose metabolism, gene regulation, and cell proliferation. The activity of Akt promotes growth and/or proliferation of muscle cells, resulting in increased muscle weight in animals where myostatin signaling is inhibited. Similarly, myostatin signaling in bone-forming cells (osteoblasts) inhibits bone deposition. In these cells, myostatin signaling promotes the expression of several bone regulatory factors, such as sclerostin, DKK1, and RANKL, and suppresses other miRNAs involved in in osteoblast development. These combined effects prevent the differentiation of osteoblasts and, consequently, the formation of new bone.

In some embodiments, type 1 receptor and/or type 2 receptor signaling is inhibited specifically in myofibers. Myofibers, also referred to as myocytes or muscle cells, are cells present in muscle tissue, including skeletal muscle, smooth muscle, and cardiac muscle. Targeted inhibition of myostatin signaling in myofibers thus promotes muscle growth without undesired side effects that may result from inhibition of type 1 receptor signaling and/or type 2 receptor signaling in other cell types or tissues. In some embodiments, type 1 receptor and/or type 2 receptor signaling is inhibited specifically in osteoblasts. Osteoblasts are cells present in bones that synthesize collagen, osteocalcin, and osteopontin, which together form the organic matrix of bone. Inhibiton of myostatin signaling specifically in osteoblasts promotes bone deposition, while similarly avoiding the undesired side effects of signaling inhibiton in other cell types or tissues. Targeted inhibition in myofibers and/or osteoblasts may be achieved, for example, by conditionally expressing agents such as antisense oligonucleotides (ASOs), RNAi molecules (e.g., shRNA, siRNA, or miRNA), and/or programmable nuclease-based gene editing molecules (e.g., CRISPR/Cas9/gRNAs, TALE/TALENs, and ZFNs) in myofibers and/or osteoblasts.

Some aspects of the present disclosure provide methods of administering to a subject an agent or composition that inhibits signaling by receptors. “Signaling,” as used herein, is the process by which information about the state of a biological system, such as the presence, absence, or amount of a substance, is detected and processed by a cell or organism. A “receptor” is a protein that can detect and transduce a biological signal, such as the presence of a ligand or, more generally, associate with a ligand. Receptors may be present outside of a cell (soluble receptors), attached to the exterior surface of a cell (extracellular receptors), embedded in the plasma membrane of a cell (transmembrane receptors), attached to the interior surface of a cell (intracellular receptors), or contained within the cytoplasm (cytoplasmic receptors and/or adaptor proteins). Receptors may contain multiple domains, located outside of the cell (extracellular domain), within the plasma membrane (transmembrane domain), and/or inside the cell (intracellular or cytoplasmic domain).

Receptors may associate with, and thereby detect the presence of, a ligand. A “ligand” is a substance, such as a protein, that associates with a receptor or other biomolecule. Association between a ligand and receptor generally occurs via intramolecular forces such as hydrogen bonds, ionic bonds, and Van der Waals forces. Non-limiting examples of ligands include carbohydrates, lipids, small molecules, macromolecules, nucleic acids, peptides and neurotransmitters. Association between a ligand and receptor may result in a biological process that does not occur when the receptor is not associated with the ligand, such as signal transduction. As used herein, “signal transduction” refers to a process in which an extracellular signal is transmitted to the intracellular environment. The extracellular signal may be the presence of a ligand, or the association of a ligand with a receptor. Signal transduction may occur through phosphorylation, dephosphorylation, conformational changes, and/or other chemical modifications of the receptor following association of a ligand with a receptor. Such modifications may result in the association between the ligand-bound receptor and other receptors, and/or the association of other intracellular proteins with intracellular or transmembrane domains of the receptor.

### Inhibitory Agents

Compositions described herein may contain inhibitors of type 1 receptor or type 2 receptor signaling. As used herein, an “inhibitor” refers to an agent or a combination of agents that inhibit(s) signaling of a particular receptor. An inhibitor may specifically inhibit signaling by a receptor. “Specific inhibition,” as used herein, refers to the action of an agent that prevents (no measurable activity) or reduces (e.g., by at least 10% relative to a control) the function of a given protein, such as by preventing signal transduction by a protein association of the protein with a ligand, which occurs more frequently than the action of the same agent against other proteins. An inhibitor may inhibit protein function (activity) by acting directly or indirectly on the protein, or by modifying (e.g., preventing) expression of the protein by modifying translation of the protein or transcription of the DNA/mRNA encoding the protein. A reduction in protein activity may be a reduction of at least 10%, at least 20%, at least 30%, at least 40%, at least 50%, at least 60%, at least 70%, at least 80%, at least 90%, at least 95%, at least 98%, or 100% (no measurable activity), relative to a control (e.g., protein activity in the absence of the inhibitor).

In some embodiments, the inhibitor associates with (e.g., binds to) the receptor, preventing other ligands from associating with the same receptor (competitive inhibition), but this association does not trigger signal transduction, and thus the presence of the inhibitor reduces the signaling activity of the receptor. Inhibition may occur by specific binding. “Specific binding,” as used herein, refers to the non-random association of an agent with a given protein, with the agent showing significantly less affinity for other proteins. Binding affinity between an agent a given protein may be quantified as the dissociation constant (K_{D}) of the interaction between the agent and protein using methods known in the art. The binding affinity of an agent to one protein relative to another may be evaluated by measuring the K_{D} values of the agent's interaction with each protein and comparing them using statistical methods that are known in the art (e.g., Student's t-test, ANOVA, regression models). An agent is said to bind to a given protein with significantly more affinity relative to another protein if 1) the K_{D} of the agent's interaction with the given protein is lower than the K_{D} of the agent's interaction with the other protein, and 2) a statistical test or model determines that the probability of the results being due to chance is less than 0.1, less than 0.05, or less than 0.01.

In some embodiments, the inhibitor is a soluble receptor (or “decoy receptor”) that associates with (e.g., binds to) the same ligand with which the targeted receptor (e.g., type 1 and/or type 2 receptor) associates. As the soluble receptor is not associated with a cell, its association with ligand will not trigger signal transduction, but will reduce the concentration of ligand in an environment, such as the bloodstream, and thus reduce the frequency of signal transduction in cells due to the lower availability of ligand (indirect inhibition). In some embodiments, the soluble receptor comprises an extracellular domain of a type 1 and/or type 2 receptor. In some embodiments, the soluble receptor comprises an (at least one) extracellular domain of ALK4, ALK5, ACVR2A, ACVR2B, and/or TGFβRII.

In some embodiments, the soluble receptor comprises the extracellular domains of ALK4 and ALK5. In some embodiments, the soluble receptor comprises the extracellular domains of ALK4 and ACVR2A. In some embodiments, the soluble receptor comprises the extracellular domains of ALK4 and ACVR2B. In some embodiments, the soluble receptor comprises the extracellular domains of ALK5 and ACVR2A. In some embodiments, the soluble receptor comprises the extracellular domains of ALK5 and ACVR2B. In some embodiments, the soluble receptor comprises the extracellular domains of ACVR2A and ACVR2B.

In some embodiments, the soluble receptor comprises the extracellular domains of TGFβRII, ALK4, and ALK5. In some embodiments, the soluble receptor comprises the extracellular domains of TGFβRII, ALK4, and ACVR2A. In some embodiments, the soluble receptor comprises the extracellular domains of TGFβRII, ALK4, and ACVR2B. In some embodiments, the soluble receptor comprises the extracellular domains of TGFβRII, ALK5, and ACVR2A. In some embodiments, the soluble receptor comprises the extracellular domains of TGFβRII, ALK5, and ACVR2B. In some embodiments, the soluble receptor comprises the extracellular domains of TGFβRII, ACVR2A, and ACVR2B.

A soluble receptor may also comprise additional domains (e.g., antibody Fc domain) that promote its degradation, clearance from circulation, or sequestration of the receptor and bound ligand away from other cells. See, e.g., Puolakkainen et al. BMC Musculoskelet Disord. (2017). 18(1):20.

Inhibitors, in some embodiments, associate with domains of a receptor involved in signal transduction, such that the receptor may still associate with ligand but exhibit reduced signal transduction activity.

Specific examples of agents that inhibit type 1 and type 2 receptor signaling are provided throughout the present disclosure. See, e.g., “Methods of Inhibiting MSTN and/or Activin Signaling” below.

Inhibitors may reduce or prevent expression of a receptor in a cell, thereby reducing the frequency of signal transduction due to the lower availability of receptors on or in a cell, such as an osteoblast or muscle cell. Non-limiting examples of inhibitors that act in this manner include antisense oligonucleotides, small interfering RNAs, short hairpin RNAs, microRNAs, and programmable nucleases (e.g., RNA-guided nucleases, TALENs, and ZFNs). Inhibitors may specifically target an mRNA or genomic locus encoding a desired protein. “Specifically target,” as used herein, refers to the non-random action of an agent against a particular target, such as an mRNA or genomic locus encoding a particular protein, with the agent showing significantly less action against other targets, such as an mRNAs or genomic loci encoding proteins other than the particular protein. Action of an agent against a target may be quantified by measuring the change in DNA sequence, mutation frequency, mRNA transcription, mRNA abundance, and/or protein translation that result from the addition of the agent, using methods known in the art (e.g., PCR, qRT-PCR, western blotting). The action of an agent against one target relative to another target may be evaluated by measuring the action of the agent against each target, and comparing the measurements using statistical methods that are known in the art (e.g., Student's t-test, ANOVA, regression models). An agent is said to have significantly more action against a given target relative to another target if 1) the magnitude of the action as measured is higher against the given target than another target, and 2) a statistical test or model determines that the probability of the results being due to chance is less than 0.1, less than 0.05, or less than 0.01.

In some embodiments, an inhibitor is an antisense oligonucleotide (ASO). An ASO is a single-stranded DNA or RNA oligonucleotide that is complementary to a target sequence. An oligonucleotide is complementary to a target sequence if the oligonucleotide binds to a nucleic acid comprising the target sequence, forming a nucleic acid that is at least partially double-stranded through hydrogen bonds between base pairs on the oligonucleotide and target sequence. An oligonucleotide is most complementary to a sequence when the oligonucleotide comprises a sequence of bases that form canonical Watson-Crick base pairs (i.e., A-U, A-T, C-G) with the target sequence, in reverse order relative to the order of bases in the target sequence. Binding of an antisense oligonucleotide to an mRNA target can interfere with normal cellular processing of the mRNA, and therefore expression of the encoded protein, through multiple mechanisms. For example, nucleases of the ribonuclease H (RNase H or RNH) family hydrolyze the phosphodiester bond between nucleotides in the RNA component of a DNA/RNA hybrid nucleic acid, in which a single-stranded DNA sequence is hybridized with a single-stranded RNA sequence. An mRNA bound by an antisense DNA oligonucleotide may thus be cleaved by RNase H, thereby preventing translation of the mRNA into an encoded protein. ASOs may also modulate gene expression by interfering with the formation of a 5′ cap on mRNA, altering the splicing process (splice-switching), and hindering translation by ribosomes through steric hindrance. See, e.g., Rinaldi et al. Nat Rev Neurol. (2018). 14(1):9-21.

In some embodiments, an inhibitor is a small interfering RNA. A small interfering RNA (siRNA), also known as short interfering RNA or silencing RNA, is a double-stranded RNA (dsRNA) that contains one strand that is complementary to a chosen target sequence (guide strand), and one strand that is complementary to the guide strand (passenger strand). The protein Argonaute (Ago) associates with the dsRNA, after which the guide strand is integrated into the RNA-induced signaling complex (RISC), and the passenger strand is degraded. The guide strand, along with the associated proteins of the RISC, then associates with a target mRNA containing a complementary sequence to the guide strand, and the Argonaute protein of the RISC cleaves the target mRNA, thereby preventing translation of the target mRNA and reducing the amount of encoded protein in the cell. RISC may also inhibit the translation of mRNAs containing a sequence complementary to a guide RNA by preventing the addition of a 5′ cap, removing the 3′ poly(A) tail, or blocking the interaction of ribosomes with mRNA by steric hindrance. See, e.g., Wittrup et al. Nat Rev Genet. (2015). 16(9):543-552.

In some embodiments, an inhibitor is a short hairpin RNA. A short hairpin RNA (shRNA), also known as a small hairpin RNA, is a single-stranded RNA that contains a hairpin, or loop, structure of unpaired bases. The hairpin is formed when a sequence in the RNA hybridizes with another sequence in the same RNA molecule through Watson-Crick base pairing, with the hairpin comprising the unpaired bases between the two complementary sequence. shRNAs can be cleaved by the enzyme Dicer, resulting in the formation of double-stranded siRNAs that can inhibit gene expression as described previously. See, e.g., Moore et al. Methods Mol Biol. (2010). 629:141-158.

In some embodiments, an inhibitor is a microRNA. A microRNA (miRNA) is a small RNA molecule that can function in RNA interference and gene regulation. A miRNA is generated from a longer RNA precursor known as primary miRNA (pri-miRNA), which is cleaved by the enzyme Drosha to form a shorter precursor miRNA (pre-miRNA) containing a hairpin. This pre-miRNA is then processed by Dicer to form a double-stranded intermediate RNA similar to a double-stranded siRNA, which can be integrated into a RISC and inhibit the expression of mRNAs containing a target sequence as described previously. See, e.g., O'Brien et al. Front Endocrinol. (2018). 9:402.

In some embodiments, an inhibitor is a programmable nuclease. A programmable nuclease is a protein that can be directed to cleave nucleic acids at a target site, such as a specific nucleotide sequence. Programmable nucleases cut at or near target sequences, forming DNA double-stranded breaks. Cutting at a target sequence means cutting within the nucleotide sequence that is recognized by the programmable nuclease. Cutting near a target sequence may be within 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, or 20 nucleotides.

In some embodiments, the programmable nuclease is a zinc-finger nuclease. A zinc-finger nuclease (ZFN) is an endonuclease that can be programmed to cut specific sequences of DNA. ZFNs are composed of a zinc-finger DNA-binding domain and a nuclease domain. The DNA-binding domains of individual ZFNs generally contain 3-6 individual zinc finger repeats that recognize 9-18 nucleotides. For example, if the zinc finger domain perfectly recognizes a three base pair sequence, then a three-zinc finger array can be generated to recognize a nine-base pair target DNA sequence. Because individual zinc fingers recognize relatively short (e.g., three base pairs) target DNA sequences, ZFNs with 4, 5, or 6 zinc finger domains are typically used to minimize off-target DNA cutting. Non-limiting examples of zinc finger DNA-binding domains that may be used with methods of the present disclosure include Zif268, Gal4, HIV nucleocapsid protein, MYST family histone acetyltransferases, myelin transcription factor Myt1, and suppressor of tumorigenicity protein 18 (ST18). A ZFN may contain homogeneous DNA binding domains (all from the same source molecule) or a ZFN may contain heterogeneous DNA binding domains (at least one DNA binding domain is from a different source molecule).

Zinc finger DNA-binding domains work in concert with a nuclease domain to form a zinc finger nucleases (ZFNs) that cut target DNA (e.g., breakpoint junction). The nuclease cuts the DNA in a non-sequence specific manner after being recruited to the target DNA (e.g., breakpoint junction) by the zinc fingers DNA-binding domains. The most widely-used ZFN is the type 2 restriction enzyme FokI, which forms a heterodimer before producing a double-stranded break in the DNA. Thus, two ZFN proteins must bind to opposite strands of DNA to create the FokI heterodimer and form a double-stranded break, reducing off-target DNA cleavage events (see, e.g., Kim, et al., Proc Natl Acad Sci USA, 1996, 93(3): 1156-1160). Additionally, ZFNs may be nickases that only cleave one strand of the double-stranded DNA. By cleaving only one strand, the DNA is more likely to be repaired by error-free HR as opposed to error-prone NHEJ (see, e.g., Ramirez, et al. Nucleic Acids Research, 40(7): 5560-5568). Non-limiting examples of nucleases that may be used with methods in this disclosure include FokI and DNaseI.

In some embodiments, the programmable nuclease is a transcription activator-like effector nuclease. A transcription activator-like effector nuclease (TALEN) is an endonuclease that can be programmed to cut specific sequences of DNA. TALENs are composed of transcription activator-like effector (TALE) DNA-binding domains, which recognize single target nucleotides in the TNA, and transcription activator-like effector nucleases (TALENs) which cut the DNA at or near the target nucleotide. Transcription activator-like effectors (TALEs) found in bacteria are modular DNA binding domains that include central repeat domains made up of repetitive sequences of residues (see, e.g., Boch J. et al. Annual Review of Phytopathology 2010; 48: 419-36; Boch J Biotechnology 2011; 29(2): 135-136). The central repeat domains contain multiple repeat regions, with certain amino acids of the repeat region, known as the repeat variable diresidue (RVD) determining the nucleotide specificity of the TALE (see, e.g., Moscou M J et al. Science 2009; 326 (5959): 1501; Juillerat A et al. Scientific Reports 2015; 5: 8150). Unlike SF DNA sensors, TALE-based sequence detectors can recognize single nucleotides. Combining multiple repeat regions may produce sequence-specific synthetic TALEs (see, e.g., Cermak T et al. Nucleic Acids Research 2011; 39 (12): e82). Non-limiting examples of TALEs include IL2RG, AvrB s, dHax3, and thXoI.

In some embodiments, the programmable nuclease is an RNA-guided nuclease. RNA-guided nucleases are directed to a target sequence through the use of a guide RNA (gRNA) that is complementary to the target sequence. Thus, a specific guide RNA may be utilized to direct the activity of the RNA-guided nuclease such that only the target sequence is cleaved.

In some embodiments, the RNA-guided nuclease is a Clustered Regularly Interspace Palindromic Repeats-Associated (CRISPR/Cas) nuclease. CRISPR/Cas nucleases exist in a variety of bacterial species, where they recognize and cut specific DNA sequences. The CRISPR/Cas nuclease are grouped into two classes. Class 1 systems use a complex of multiple CRISPR/Cas proteins to bind and degrade nucleic acids, whereas Class 2 systems use a large, single protein for the same purpose. A CRISPR/Cas nuclease as used herein may be selected from Cas9, Cas10, Cas3, Cas4, C2c1, C2c3, Cas13a, Cas13b, Cas13c, and Cas14 (see, e.g., Harrington, L. B. et al., Science, 2018 (DOI: 10.1126/scienceaav4294)).

Non-limiting examples of bacterial CRISPR/Cas9 nucleases for use herein include Streptococcus thermophilus Cas9, Streptococcus thermophilus Cas10, Streptooccus thermophilus Cas3, Staphylococcus aureus Cas9, Staphylococcus aureus Cas10, Staphylococcus aureus Cas3, Neisseria meningitidis Cas9, Neisseria meningitidis Cas10, Neisseria meningitidis Cas3, Streptococcus pyogenes Cas9, Streptococcus pyogenes Cas10, and Streptococcus pyogenes Cas3. Other variant endonucleases may be used.

In some embodiments, an RNA-guided nuclease is a CRISPR-associated endonuclease in Prevotella and Francisella 1 (Cpf1). Cpf1 is a bacterial endonuclease similar to Cas9 nuclease in terms of activity. However, Cpf1 only requires a short (˜42 nucleotide) gRNA, while Cas9 requires a longer (˜100 nucleotide) gRNA. Additionally, Cpf1 cuts the DNA 5′ to the target sequence and leaves blunted ends, while Cas9 leaves sticky ends with DNA overhangs. Cpf1 proteins from Acidaminococcus and Lachnospiraceae bacteria efficiently cut DNA in human cells in vitro. In some embodiments, the RNA-guided nuclease is Acidaminococcus Cpf1 or Lachnospiraceae Cpf1, which require shorter gRNAs than Cas nuclease proteins.

In some embodiments, inhibitors such as antisense oligonucleotides (ASOs), RNAi molecules (e.g., shRNA, siRNA, or miRNA), and/or programmable nuclease-based gene editing molecules (e.g., CRISPR/Cas9/gRNAs, TALE/TALENs, and ZFNs) are conditionally expressed in myofibers and/or osteoblasts. “Conditional expression,” as used herein, refers to the non-random expression of a gene, transcription or generation of an RNA, or production of a protein in a particular environment (e.g. a specific cell type, or in response to a signal) with significantly less expression in other environments (e.g., other cell types or in the absence of a signal). Expression may be quantified by measuring mRNA transcription, mRNA abundance, protein translation, and/or protein abundance using methods that are known in the art (e.g., qRT-PCR, Western blotting, immunoassays). The frequency of expression in one environment relative to another may be evaluated by measuring expression in both environments and comparing the measurements using statistical methods that are known in the art (e.g., Student's t-test, ANOVA, regression models). An agent is said to have significantly more expression in a given environment relative to another environment if 1) the magnitude of expression as measured is higher in the given environment than in another environment, and 2) a statistical test or model determines that the probability of the results being due to chance is less than 0.1, less than 0.05, or less than 0.01. This may be achieved by operably linking the gene to be expressed to a promoter that is active in the desired environment, but not other environments. A promoter is said to be operably linked to a gene if the promoter controls the degree to which the gene is expressed. Conditional expression of a gene (e.g., for the production of an ASO, RNAi molecule, and/or programmable nuclease) in myofibers, for example, may be achieved through the use of the promoter. Other non-limiting examples of promoters active in muscle cells include the promoter regions of TnIs, TnCf, and skAct. See, e.g., Corin et al. Proc Natl Acad Sci USA. (1995). 92(13): 6185-6189. Conditional expression of a gene in osteobalsts, for example, may be achieved using the promoter region of any gene that is specifically active in osteoblasts. Non-limiting examples of promoters active in osteoblasts include the promoter regions of the COL1A2 and OCN genes.

### Methods of Inhibiting TGFIβ Family Signaling

The present disclosure provides, in some embodiments, methods of administering to a subject a composition containing inhibitors of type 1 receptor and/or type 2 receptor signaling. Type I receptors and type 2 receptors are receptors that associate with (e.g., bind to) activin or activin-like ligands. Non-limiting examples of activin-like ligands include myostatin, TGFβ1, TGFβ2, TGFβ3, inhibin α, inhibin βA, inhibin βB, inhibin PC, BMP1, BMP2, BMP3, BMP4, BMP5, BMP6, BMP7, BMP8a, BMP8b, BMP9, BMP10, BMP11, BMP12, BMP13, BMP14, BMP15, GDF-1, GDF-2, GDF-3, GDF-4, GDF-5, GDF-6, GDF-7, GDF-8, GDF-9, GDF-10, GDF-11, GDF-12, GDF-13, GDF-14, GDF-15, nodal, and anti-müllerian hormone (AMH). See, e.g., Tsuchida et al. Cell Commun Signal. (2009). 7:15; Loomans et al. Am J Cancer Res. (2016). 6(11): 2431-2447.

Signaling through type 1 receptors and/or type 2 receptors occurs when activin, or an activin-like ligand such as myostatin, binds to a type 2 receptor, such as ACVR2A or ACVR2B, and the type 2 receptor then forms a complex with a type 1 receptor, such as ALK4 or ALK5. This complex then recruits and phosphorylates proteins such as SMAD2 or SMAD3. Phosphorylated SMAD2 and SMAD3 dimerize, then form a complex with SMAD4. This SMAD2-SMAD3-SMAD4 complex then translocates to the nucleus, where it regulates gene expression. Signal transduction may also include the inhibition of Akt, a serine/threonine kinase. Active Akt phosphorylates Forkhead transcription factors (FoxO proteins), and thus inhibition of Akt activity results in the generation of dephosphorylated FoxO as phosphate groups are lost from phosphorylated FoxO and not replaced. Dephosphorylated FoxO translocates to the nucleus and activate transcription of E3 ubiquitin ligases MuRF1 and Atrogin 1. These E3 ubiquitin ligases mark muscle contractile proteins for degradation by the proteasome, and so inhibition of Akt by myostatin, type 1 receptor(s), and/or type 2 receptor(s) can result in a reduction in muscle weight. See, e.g., Han et al. Curr Opin Support Palliat Care. (2011). 5(4):334-341. E3 ubiquitin ligases similarly mark proteins such as the osteocalcin required for bone formation, resulting in proteasomal degradation, decreased bone deposition, and reduced bone mineral density. See, e.g., Xi et al. J Recept Signal Transduct Res. 2015. 35(6):640-645.

### Type 1 Receptor Inhibition

Some aspects of the present disclosure provide methods of administering inhibitors of type 1 receptors signaling to a subject. Type I receptors are required for signaling by activin and other activin-like ligands such as myostatin. Following association between a ligand and a type 2 receptor, the type 2 receptor associates with a type 1 receptor, after which signal transduction occurs. The data provided herein show that type 1 receptors are functionally redundant with each other, such that the deletion of one type 1 receptor is insufficient to abrogate myostatin and/or activin signaling in a subject, but the deletion of multiple type 1 receptors reduces myostatin and/or activin signaling and thus increases muscle weight, improves blood glucose, reduces body fat content, and increases bone density in a subject.

In some embodiments, the type 1 receptor is ALK4, also referred to as activin receptor type-1B (ACVR1B). ALK4 is encoded by the ACVR1B gene, and acts as a transducer of signals activin or activin-like ligands. Non-limiting examples of ligands that interact with ALK4 are myostatin, activin A, activin B, activin AB, nodal, GDF-1, GDF-3, GDF-8, GDF-11, BMP11, TGFβ1, TGFβ2, TGFβ3 and Vg1. Non-limiting examples of ALK4 inhibitors (i.e. agents that inhibit signaling through ALK4) include follistatin, inhibin A, inhibin B, left-right determination factor 1, left-right determination factor 2, A 83-01, SB-431542, SB-505124, EW-7197, K02288, LDN-212854, LY-364947, LY-2157299, Galunisertib, GW-6604, SD-208, AZ12799734, Vactosertib, EW-7195, TP-008, E616452, SB525334, SJN2511, AZ12601011, GW 788388, and SM16. See, e.g., Cui et al. Mol Med Rep. (2019). 19(6): 5053-5062; Loomans et al. Am J Cancer Res. (2016). 6(11): 2431-2447; Chapman et al. Mol Endocrinol. (2001). 15(4): 668-679; Fields et al. Expert Opin Investig Drugs. (2013) 22(1):87-101.

An example of a DNA sequence for human ALK4 is given by Accession No. NM 020328.4 and is reproduced as SEQ ID NO: 1.

An example of an amino acid sequence for human ALK4 is given by Accession No. P36896-1 and is reproduced as SEQ ID NO: 2.

In some embodiments, the type 1 receptor is ALK5, also referred to as transforming growth factor β receptor I or TGFβRI. ALK5 is encoded by the TGFBR1 gene, and acts as a receptor for activins, which belong the TGFβ superfamily of signaling ligands. Non-limiting examples of ligands that interact with ALK5 are myostatin, avotermin, GDF-10, BMP3B, GDF-11, BMP11, TGFβ1, TGFβ2, TGFβ3. Non-limiting examples of ALK5 inhibitors (i.e. agents that inhibit signaling through ALK5) include follistatin, fresolimumab, lerdelimumab, metelimumab, A 83-01, D-4476, GW-788388, LY-364947, LY-2109761, Galunisertib (LY-2157299), R-268712, RepSox (E-616452, SJN-2511), SB-431542, SB-505124, SB-525334, SD-208, EW-7197, K02288, LDN-212854, GW-6604, AZ12799734, Vactosertib, EW-7195, TP-008, AZ12601011, and SM16. See, e.g., Cui et al. Mol Med Rep. (2019). 19(6): 5053-5062; Loomans et al. Am J Cancer Res. (2016). 6(11): 2431-2447; Chapman et al. Mol Endocrinol.

(2001). 15(4): 668-679; Fields et al. Expert Opin Investig Drugs. (2013) 22(1):87-101.

An example of a DNA sequence for human ALK5 is given by Accession No. NM 001130916.3 and is reproduced as SEQ ID NO: 3.

An example of an amino acid sequence for human ALK5 is given by Accession No. P36897 and is reproduced as SEQ ID NO: 4.

### Type 2 Receptor Inhibition

Some aspects of the present disclosure provide methods of administering inhibitors of type 2 receptors signaling to a subject. A type 2 receptor, after association with activin or an activin-like ligand such as myostatin, can dimerize with a type 1 receptor, resulting in signal transduction. The data provided herein show that type 2 receptors are functionally redundant with each other, such that the deletion of one type 2 receptor is insufficient to abrogate myostatin signaling in a subject, but the deletion of multiple type 2 receptors reduces myostatin signaling and thus increases muscle weight, improves blood glucose, reduces body fat content, and increases bone density in a subject.

In some embodiments, the type 2 receptor is ACVR2A, also referred to as activin receptor type-2A or ACVR2. ACVR2A is encoded by the ACVR2A gene, and acts as a receptor for activins, which belong the TGFβ superfamily of signaling ligands. Non-limiting examples of ligands that interact with ACVR2A are myostatin, activin A, activin B, activin AB, BMP2, BMP4, BMPS, BMP6, BMP7, BMP8A, BMP8B, BMP11, BMP12, BMP13, BMP14, BMP15,

GDF-1, GDF-3, GDF-5, GDF-6, GDF-7, GDF-9, GDF-8, GDF-11, GDF-15, dibotermin alfa, eptotermin alfa, nodal, and radotermin. Non-limiting examples of ACVR2A inhibitors (i.e. agents that inhibit signaling through ACVR2A) include follistatin, inhibin A, inhibin B, left-right determination factor 1, left-right determination factor 2, sotarercept, CZC24758, dorsomorphin, LDN-193189, bimagrumab, CDD861, BYM338, ACVR2A/Fc, and ACVR2-ECD. See, e.g., Goh et al. J Biol Chem (2017). 292(33): 13809-13822.

An example of a DNA sequence of human ACVR2A is given by Accession No. NM_001616.5 and is reproduced as SEQ ID NO: 5.

An example of an amino acid sequence of human ACVR2A is given by Accession No. P27037 and is reproduced as SEQ ID NO: 6.

In some embodiments, the type 2 receptor is ACVR2B, also referred to as activin receptor type-2B. ACVR2B is encoded by the ACVR2B gene, and acts as a receptor for activins, which belong the TGFβ superfamily of signaling ligands. Non-limiting examples of ligands that interact with ACVR2B are myostatin, activin A, activin B, activin AB, BMP1, BMP2, BMP3, BMP3A, BMP4, BMP5, BMP6, BMP7, BMP8A, BMP8B, BMP11, BMP12, BMP13, BMP14, BMP15, GDF-1, GDF-3, GDF-5, GDF-6, GDF-7, GDF-9, GDF-8, GDF-11, GDF-15, dibotermin alfa, eptotermin alfa, nodal, and osteogenin, and radotermin. Non-limiting examples of ACVR2B inhibitors (e.g., agents that inhibit signaling through ACVR2B) include follistatin, inhibin A, inhibin B, left-right determination factor 1, left-right determination factor 2, ramatercept, CZC24758, dorsomorphin, LDN-193189, bimagrumab, CDD861, BYM338, DLK1, RAP-031, ACVR2B/Fc, and ACVR2B-ECD. See, e.g., Formicola et al. Front Physiol. (2018). 9:515; Sako et al. J Biol Chem. (2010). 285(27): 21037-21048; Goh et al. J Biol Chem. (2017) 292(33): 13809-13822.

An example of a DNA sequence of human ACVR2B is given by Accession No. NM_001106.4 and is reproduced as SEQ ID NO: 7.

An example of an amino acid sequence of human ACVR2B is given by Accession No. Q13705 and is reproduced as SEQ ID NO: 8.

In some embodiments, the type 2 receptor is transforming growth factor β receptor II (TGFβRII). TGFβRII is encoded by the TGFBR2 gene, and acts as a receptor for TGFβ1, TGFβ2, and TGFβ3, which belong the TGFβ superfamily of signaling ligands. Non-limiting examples of TGFβRII inhibitors (e.g., agents that inhibit signaling through TGFβRII) include follistatin, fresolimumab, lerdelimumab, metelimumab, ITD-1 DMH-2, LY-364947, LY-2109761, galunisertib (LY-2157299), compound 13a (PMID: 23639540), compound 13d (PMID: 23639540), and compound 15b (PMID: 16539403). See, e.g., Willems et al. Cell Stem Cell. (2012). 11(2): 242-252; Neuzillet et al. Pharmacol Ther. (2015) 147:22-31.

An example of a DNA sequence of human TGFBR2 is given by Accession No. NM_001024847.2 and is reproduced as SEQ ID NO: 9.

An example of an amino acid sequence of human TGFβRII is given by Accession No. P37173 and is reproduced as SEQ ID NO: 10.

### Inhibition of TGFβ Signaling

Some aspects of the present disclosure provide methods of administering inhibitors of TGFβ signaling to a subject. The transforming growth factor beta (TGFβ) superfamily is a family of multifunctional cytokines that regulate growth and developmental processes. TGFβ is present in three different isoforms, TGFβ1, TGFβ2, and TGFβ3, which are encoded by the genes TGFB1, TGFB2, and TGFB3, respectively. TGFβ1 regulates the proliferation, differentiation, and activation of multiple cell types, including T cells, B cells, myeloid cells, muscle cells, osteoblasts, and osteoclasts. TGFβ2 modulates multiple processes such as cellular metabolism, embryonic development, and tumor suppression. TGFβ3 regulates cellular adhesion, mammalian development, and wound healing. The three isoforms TGFβ1, TGFβ2, and TGFβ3 interact with ALK5 and TGFβRII. Inhibition of TGFβRII signaling may thus be achieved through the use of an agent or combination of agents that bind(s) to TGFβRII or the use of an agent or combination of agents that bind(s) to TGFβ1, TGFβ2, and/or TGFβ3. Similarly, inhibition of TGFβ1, TGFβ2, and/or TGFβ3 signaling may be achieved through the use of an agent or combination of agents that bind(s) to TGFβ1, TGFβ2, and/or TGFβ3, or the use of an agent or combination of agents that bind(s) to TGFβRII. The TGFβ superfamily also includes myostatin and activin A, which act as negative regulators of skeletal muscle growth, osteoblast differentiation, and bone deposition. The data provided herein show that inhibiting myostatin signaling and/or signaling through type 1 and/or type 2 receptors, either through targeted inhibition of myostatin, or receptors that associate with myostatin and other TGFβ cytokines, results in increased muscle mass and bone mineral density in a subject. See, e.g., Hata et al. Cold Spring Harb Perspect Biol. (2016). 8(9):a022061.

### Compositions

Some aspects of the present disclosure provide compositions (e.g., for use in a method of treatment) comprising an agent or combination of agents that inhibit signaling through type 1 receptors and/or type 2 receptors. In some embodiments, the composition comprises an agent or combination of agents that inhibit signaling through ALK4, ALK5, ACVR2A, ACVR2B, and/or TGFβRII. In some embodiments, agents that inhibit signaling through ALK4, ALK5, ACVR2A, ACVR2B, and/or TGFβRII are co-formulated (present in the same composition). In some embodiments, the composition is administered in an effective amount.

An effective amount, which may also be referred to as a therapeutically effective amount, refers to the amount (e.g., dose) at which a desired clinical result (e.g., muscle growth and/or bone deposition) is achieved in a subject. An effective amount is based, at least in part, on the target tissue, target cell type, means of administration, physical characteristics of the inhibitor, other components of the composition, and other determinants, such as age, body weight, height, sex and general health of the subject. A subject may be a mammal, such as a human, a non-human primate (e.g., Rhesus monkey, chimpanzee), or a rodent (e.g., a mouse or a rat). In some embodiments, the subject is a human subject.

In some embodiments, the subject has a disease associated with myostatin signaling. In some embodiments, the subject has a disease associated with type 1 and/or type 2 receptor signaling. In some embodiments, the subject has a disease associated with type 1 and/or type 2 receptor signaling in muscle cells. In some embodiments, the subject has Duchenne muscular dystrophy, facioscapulohumeral muscular dystrophy, inclusion body myositis, muscle atrophy, spinal muscle atrophy, age-related sarcopenia, Charcot-Marie-Tooth disease, cachexia, chronic obstructive pulmonary disease, kidney disease, or cancer. In some embodiments, the subject has a disease associated with type 1 and/or type 2 receptor signaling in osteoblasts. In some embodiments, the subject has osteoporosis, Cushing's syndrome, pituitary disorders, and hyperthyroidism.

Some aspects of the present disclosure provide compositions and methods of use for treating Duchenne muscular dystrophy (DMD). DMD is a genetic disorder characterized by progressive muscle degeneration and weakness due to a mutation in the DMD gene, which encodes the dystrophin protein, a critical protein for muscle cell function.

Some aspects of the present disclosure provide compositions and methods of use for treating facioscapulohumeral muscular dystrophy (FSHD). FSHD is a genetic disorder associated with mutations that alter expression of the DUX4 gene, resulting in weakening of the muscles of the face, surrounding the scapula, and of the upper arm.

Some aspects of the present disclosure provide compositions and methods of use for treating inclusion body myositis (IBM). IBM is an inflammatory muscle disease of unknown cause characterized by progressive muscle degeneration due to the infiltration of muscle tissue by immune cells, deposition of abnormal proteins, and filamentous inclusions in muscle fibers.

Some aspects of the present disclosure provide compositions and methods of use for treating muscle atrophy. Muscle atrophy is the loss of skeletal muscle mass due to one of many potential causes, including disuse, immobility, aging, malnutrition, or injury.

Some aspects of the present disclosure provide compositions and methods of use for treating spinal muscular atrophy (SMA). SMA is a neuromuscular disorder that results in the loss of motor neurons and progressive degeneration and weakening of muscles, especially those of the arms, legs, and respiratory system.

Some aspects of the present disclosure provide compositions and methods of use for treating age-related sarcopenia. Age-related sarcopenia is the degenerative loss of muscle mass and strength that occurs with aging, involving reduction in the number of muscle fibers and loss of muscle regeneration activity.

Some aspects of the present disclosure provide compositions and methods of use for treating Charcot-Marie-Tooth disease (CMT). CMT is a genetic disorder of the nervous system characterized by the progressive loss of muscle tissue in multiple parts of the body.

Some aspects of the present disclosure provide compositions and methods of use for treating cachexia. Cachexia is a complex syndrome resulting in loss of muscle tissue due to increased proteolysis, decreased protein synthesis, and signaling of TGFβ and activin in muscle cells. Cachexia is often associated with chronic obstructive pulmonary disease (an inflammatory disease that blocks airflow to and from the lungs, impairing breathing), kidney disease (disruption in normal kidney function), and cancer (unregulated cell growth resulting in tumor formation and/or disruption of healthy physiology in the affected organ(s)).

Some aspects of the present disclosure provide compositions and methods of use for treating osteoporosis. Osteoporosis is skeletal disorder characterized by low bone mineral density and deterioration of bone tissue. Individuals with osteoporosis experience fragility in deteriorating bones, and are at increased risk for fractures, particularly in vertebrae, arms, and hips.

Some aspects of the present disclosure provide compositions and methods of use for treating Cushing's syndrome. Cushing's syndrome is a condition resulting from prolonged exposure to glucocorticoid hormones, such as cortisol. Multiple signs and symptoms are associated with Cushing's syndrome, including weak bones, which present an increased risk for fractures.

Some aspects of the present disclosure provide compositions and methods of use for treating a pituitary disorder. Pituitary disorders are associated with abnormal activity of the pituitary gland, and consequently abnormal development. Hormone imbalances associated with pituitary disorders can hinder the accumulation of bone mineral density during development and puberty, and thus place individuals with pituitary disorders at greater risk for bone weakness and fractures.

Some aspects of the present disclosure provide compositions and methods of use for treating hyperthyroidism. Hyperthyroidism is a condition characterized by overproduction of the thyroid hormone thyroxine. Hyperthyroidism is associated with accelerated bone remodeling by osteoclasts, leading to reduced bone density, osteoporosis, and increased risk of fractures.

In some embodiments, a composition is a pharmaceutical composition. A pharmaceutical composition is a combination of an (at least one) active agent, such as an ALK4 inhibitor, ALK5 inhibitor, ACVR2A inhibitor, ACVR2B inhibitor, and/or TGFβRII inhibitor, with an excipient, inert or active, making the composition especially suitable for therapeutic use in vivo or ex vivo. A pharmaceutically acceptable excipient can also be incorporated in a formulation and can be any excipient (e.g., carrier) known in the art. Non-limiting examples include water, lower alcohols, higher alcohols, polyhydric alcohols, monosaccharides, disaccharides, polysaccharides, hydrocarbon oils, fats and oils, waxes, fatty acids, silicone oils, nonionic surfactants, ionic surfactants, silicone surfactants, and water-based mixtures and emulsion-based mixtures of such carriers.

Any pharmaceutically acceptable excipients are known in the art (see, e.g., Remington, The Science and Practice of Pharmacy (21st Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and triglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, and salicylate. Each pharmaceutically acceptable excipients used in a pharmaceutical composition of the invention must be “acceptable” in the sense of being compatible with the other ingredients of the formulation and not injurious to the subject. Excipients suitable for a selected dosage form and intended route of administration are well known in the art, and acceptable diluents or carriers for a chosen dosage form and method of administration can be determined using ordinary skill in the art.

In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with DNA or RNA (e.g., for transplantation into a subject), hyaluronidase, nanoparticle mimics and combinations thereof.

Relative amounts of the active agent, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, or at least 80% (w/w) active ingredient.

The ratio of a first inhibitor to a second inhibitor in a composition may vary. In some embodiments, the ratio of the first inhibitor to the second inhibitor is 1:1 to 1:10, or 1:1 to 1:5. For example, the ratio of ALK4 inhibitor to ALK5 inhibitor may be 1:1, 1:2, 1:3, 1:4, 1:5, 1:6, 1:7, 1:8., 1:9, or 1:10. In other embodiments, the ratio of a second inhibitor to a first inhibitor is 1:1 to 1:10, or 1:1 to 1:5. For example, the ratio of ALK5 inhibitor to ALK4 inhibitor may be 1:1, 1:2, 1:3, 1:4, 1:5, 1:6, 1:7, 1:8, 1:9, or 1:10.

The ratio of a first inhibitor to a second inhibitor in a composition may vary. In some embodiments, the ratio of the first inhibitor to the second inhibitor is 1:1 to 1:10, or 1:1 to 1:5. For example, the ratio of ACVR2A inhibitor to ALK5 inhibitor may be 1:1, 1:2, 1:3, 1:4, 1:5, 1:6, 1:7, 1:8., 1:9, or 1:10. In other embodiments, the ratio of a second inhibitor to a first inhibitor is 1:1 to 1:10, or 1:1 to 1:5. For example, the ratio of ALK5 inhibitor to ACVR2A inhibitor may be 1:1, 1:2, 1:3, 1:4, 1:5, 1:6, 1:7, 1:8, 1:9, or 1:10.

The ratio of a first inhibitor to a second inhibitor in a composition may vary. In some embodiments, the ratio of the first inhibitor to the second inhibitor is 1:1 to 1:10, or 1:1 to 1:5. For example, the ratio of ACVR2B inhibitor to ALK5 inhibitor may be 1:1, 1:2, 1:3, 1:4, 1:5, 1:6, 1:7, 1:8., 1:9, or 1:10. In other embodiments, the ratio of a second inhibitor to a first inhibitor is 1:1 to 1:10, or 1:1 to 1:5. For example, the ratio of ALK5 inhibitor to ACVR2B inhibitor may be 1:1, 1:2, 1:3, 1:4, 1:5, 1:6, 1:7, 1:8, 1:9, or 1:10.

The ratio of a first inhibitor to a second inhibitor in a composition may vary. In some embodiments, the ratio of the first inhibitor to the second inhibitor is 1:1 to 1:10, or 1:1 to 1:5. For example, the ratio of ACVR2A inhibitor to ACVR2B inhibitor may be 1:1, 1:2, 1:3, 1:4, 1:5, 1:6, 1:7, 1:8., 1:9, or 1:10. In other embodiments, the ratio of a second inhibitor to a first inhibitor is 1:1 to 1:10, or 1:1 to 1:5. For example, the ratio of ACVR2B inhibitor to ACVR2A inhibitor may be 1:1, 1:2, 1:3, 1:4, 1:5, 1:6, 1:7, 1:8, 1:9, or 1:10.

In some embodiments, the composition comprises an agent or combination of agents that inhibit(s) signaling through TGFβRII, ALK4, and ALK5. In some embodiments, the composition comprises an agent or combination of agents that inhibit(s) signaling through TGFβRII, ALK4, and ACVR2A. In some embodiments, the composition comprises an agent or combination of agents that inhibit(s) signaling through TGFβRII, ALK4, and ACVR2B. In some embodiments, the composition comprises an agent or combination of agents that inhibit(s) signaling through TGFβRII, ALK5, and ACVR2A. In some embodiments, the composition comprises an agent or combination of agents that inhibit(s) signaling through TGFβRII, ALK5, and ACVR2B. In some embodiments, the composition comprises an agent or combination of agents that inhibit(s) signaling through TGFβRII, ACVR2A, and ACVR2B.

### Methods of Treatment

Some aspects of the present disclosure provide methods for treating diseases associated with myostatin signaling by administering to a subject an agent or combination of agents that inhibit signaling through type 1 receptors and/or type 2 receptors. In some embodiments, the method comprises administering to a subject (e.g., a human subject having muscular dystrophy) a composition comprising an agent or combination of agents that inhibit signaling through ALK4, ALK5, ACVR2A, ACVR2B, and/or TGFβRII. Non-limiting examples of routes of administration include oral (e.g., tablet, capsule), intravenous, intramuscular, intraperitoneal, subcutaneous, intranasal, and intratumoral.

In some embodiments, at least two inhibitors are administered sequentially. For example, an ALK4 inhibitor) may be administered before or after (e.g., on the order of minutes, hours, or days before or after) an ALK5 inhibitor. In some embodiments, at least two inhibitors are administered concomitantly (at the same time). For example, an ALK4 inhibitor and an ALK5 inhibitor may be formulated in the same composition.

In some embodiments, at least two inhibitors are administered sequentially. For example, an ACVR2A inhibitor may be administered before or after (e.g., on the order of minutes, hours, or days before or after) an ALK5 inhibitor. As another example, an ACVR2B inhibitor may be administered before or after (e.g., on the order of minutes, hours, or days before or after) an ALK5 inhibitor. As yet another example, an ACVR2A inhibitor may be administered before or after (e.g., on the order of minutes, hours, or days before or after) an ACVR2B inhibitor.

In some embodiments, at least two inhibitors are administered concomitantly (at the same time). For example, an ACVR2A inhibitor and an ALK5 inhibitor may be formulated in the same composition. As another example, an ACVR2B inhibitor and an ALK5 inhibitor may be formulated in the same composition. As yet another example, an ACVR2A inhibitor and an ACVR2B inhibitor may be formulated in the same composition.

In some embodiments, the method comprises administering to the subject an agent or combination of agents that inhibit(s) signaling through TGFβRII, ALK4, and/or ALK5. In some embodiments, the method comprises administering to the subject an agent or combination of agents that inhibit(s) signaling through TGFβRII, ALK4, and/or ACVR2A. In some embodiments, the method comprises administering to the subject an agent or combination of agents that inhibit(s) signaling through TGFβRII, ALK4, and/or ACVR2B. In some embodiments, the method comprises administering to the subject an agent or combination of agents that inhibit(s) signaling through TGFβRII, ALK5, and/or ACVR2A. In some embodiments, the method comprises administering to the subject an agent or combination of agents that inhibit(s) signaling through TGFβRII, ALK5, and/or ACVR2B. In some embodiments, the method comprises administering to the subject an agent or combination of agents that inhibit(s) signaling through TGFβRII, ACVR2A, and ACVR2B. In some embodiments, the dose of ALK4 inhibitor, ALK5 inhibitor, ACVR2A inhibitor, ACVR2B inhibitor, and/or TGFβRII inhibitor administered to a subject is equivalent to (e.g., within 10% of), or lower than (e.g., at least 0.5-fold, at least 1-fold, at least 2-fold lower than), a control standard-of-care dose. A standard-of-care refers to a medical treatment guideline and can be general or specific. “Standard of care” specifies appropriate treatment based on scientific evidence and collaboration between medical professionals involved in the treatment of a given condition. It is the diagnostic and treatment process that a physician/ clinician should follow for a certain type of patient, illness or clinical circumstance. A standard-of-care dose as provided herein refers to the dose of ALK4 inhibitor, ALK5 inhibitor, ACVR2A inhibitor, ACVR2B inhibitor, and/or TGFβRII inhibitor that a physician/clinician or other medical professional would administer to a subject to treat or prevent cancer, while following the standard of care guideline for treating or preventing a disease associated with myostatin signaling.

In some embodiments, the dose of the inhibitor administered to a subject is a standard-of-care dose. In some embodiments, the dose of the inhibitor administered to a subject is at least 10% lower than the standard-of-care dose for the inhibitor. For example, the dose of ALK4 inhibitor administered to a subject is at least 15%, at least 20%, at least 30%, at least 40%, or at least 50% less than the standard-of-care dose for the inhibitor. In some embodiments, the dose of inhibitor administered to a subject is 10%-50%, 10%-40%, 10%-30%, 10%-20%, 20%-50%, 20%-40%, 20%-30%, 30%-50%, 30%-40%, or 40%-50% less than the standard-of-care dose for the inhibitor.

In some embodiments, the ALK4 inhibitor, ALK5 inhibitor, ACVR2A inhibitor, ACVR2B inhibitor, and/or TGFβRII inhibitor are administered in an amount effective to increase muscle weight in the subject relative to a control (e.g., baseline, prior to administration of the inhibitors, or following administration of only one of the inhibitors). The control may be a subject that is not administered a composition, or is administered a composition that does not contain any agents that inhibit signaling through ALK4, ALK5, ACVR2A, ACVR2B, and/or TGFβRII (e.g. a composition containing only a pharmaceutically acceptable excipient). The baseline may be the muscle weight of a subject as measured before administration of a composition. Muscle weight, also known as muscle mass, is the total weight or mass of muscle present in a body, organ, or particular muscle. Muscle weight may include the weight of skeletal muscle, smooth muscle, and cardiac muscle. Muscle weight may be quantified by measuring lean body mass (total body weight minus body fat weight), muscle volume (total size of muscle), and/or muscle cross-sectional area (area of the cross section of a muscle, generally at its largest point), and calculating muscle weight accordingly. Muscle weight, lean body mass, muscle volume, and muscle cross-sectional area in a subject may be determined and calculated using any number of muscle measurement methods that are known in the art and apparent to one of ordinary skill in the art (e.g., bioelectric impedance, dual-energy X-ray absorptiometry, computed tomography, and magnetic resonance imaging) (Heymsfield et al. Proc Nutr Soc. (2015). 74(4):355-366; Buckinx et al. J Cachexia Sarcopenia Muscle. (2018). 9(2):269-278). In some embodiments, muscle weight is increased by at least 3%, at least 4%, at least 5%, least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 105%, at least 110%, at least 115%, at least 120%, at least 125%, at least 130%, at least 135%, at least 140%, at least 145%, or at least 150% relative to a control or baseline. In some embodiments, muscle weight is increased by 3-300%, 3-250%, 3-100%, 3-80%, 3-50%, 3-25%, 3-10%, 3-5%, 5-300%, 5-250%, 5-100%, 5-80%, or 5-50%, 10-300%, 10-250%, 10-100%, 10-80%, or 10-50%, 25-300%, 25-250%, 25-100%, 25-80%, or 25-50% relative to a control or baseline. Non-limiting examples of muscles in which weight may be increased include the tricep, quadricep, gastrocnemius, plantaris, pectoralis, trapezius, latissimus, romboid, levator scapulae, subclavius, serratus, deltoid, teres, supraspinatus, infraspinatus, subscapularis, brachialis, anconeus, pronator, radialis, palmaris, ulnaris, pronator, flexor digitorum, flexor pollicis, extensor digitorum, extensor digitii, brachioradialis, supinator, extensor indicis, opponens, abductor, adductor, and tibialis muscles.

In some embodiments, the ALK4 inhibitor, ALK5 inhibitor, ACVR2A inhibitor, ACVR2B inhibitor, and/or TGFβRII inhibitor are administered in an amount effective to increase bone weight in the subject relative to a control (e.g., baseline, prior to administration of the inhibitors, or following administration of only one of the inhibitors). The control may be a subject that is not administered a composition, or is administered a composition that does not contain any agents that inhibit signaling through ALK4, ALK5, ACVR2A, ACVR2B, and/or TGFβRII (e.g. a composition containing only a pharmaceutically acceptable excipient). The baseline may be the muscle weight of a subject as measured before administration of a composition. Bone weight, also known as bone mineral density, is the total weight or mass of bone present in a body or particular bone. Muscle weight may include the weight of skeletal muscle, smooth muscle, and cardiac muscle. Bone weight and density in a subject may be determined and calculated using any number of muscle measurement methods that are known in the art and apparent to one of ordinary skill in the art (e.g., dual X-ray absorptiometry, quantitative CT scanning, and ultrasonography) (Sheu and Diamond. Aust Prescr. 2016. 39(2):35-39).). In some embodiments, bone weight is increased by at least 3%, at least 4%, at least 5%, least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 105%, at least 110%, at least 115%, at least 120%, at least 125%, at least 130%, at least 135%, at least 140%, at least 145%, or at least 150% relative to a control or baseline. In some embodiments, bone weight is increased by 3-300%, 3-250%, 3-100%, 3-80%, 3-50%, 3-25%, 3-10%, 3-5%, 5-300%, 5-250%, 5-100%, 5-80%, or 5-50%, 10-300%, 10-250%, 10-100%, 10-80%, or 10-50%, 25-300%, 25-250%, 25-100%, 25-80%, or 25-50% relative to a control or baseline. Non-limiting examples of bones in which bone mineral density may be increased include the occipital bone, parietal bone, frontal bone, temporal bone, sphenoid bone, ethmoid bone, mandible, humerus, scapula, clavicle, ulna, radius, carpals metacarpals, phalanges, cervical vertebrae, thoracic vertebrae, lumbar vertebrae, sacrum, coccyx, pelvis, femur, patella, tibia, fibula, tarsals, and metatarsals.

In some embodiments, the ALK4 inhibitor, ALK5 inhibitor, ACVR2A inhibitor, ACVR2B inhibitor, and/or TGFβRII inhibitor are administered in an amount effective to improve glucose metabolism in the subject relative to a control (e.g., baseline, prior to administration of the inhibitors, or following administration of only one of the inhibitors). The control may be a subject that is not administered a composition, or is administered a composition that does not contain any agents that inhibit signaling through ALK4, ALK5, ACVR2A, ACVR2B, and/or TGFβRII (e.g. a composition containing only a pharmaceutically acceptable excipient). The baseline may be the glucose metabolism of a subject as measured before administration of a composition. Glucose metabolism, as used herein, refers to the process of breaking down glucose and/or converting glucose to other molecules, as well as the rate at which these processes occur. Glucose metabolism in a subject may be measured using any number of methods that are known in the art and apparent to one of ordinary skill in the art (e.g., blood glucose concentration, insulin secretion, and magnetic resonance imaging) (Ayala et al. Dis Model Mech. (2010). 3(9-10):525-534).

In some embodiments, glucose metabolism is improved by at least 5%, least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 105%, at least 110%, at least 115%, at least 120%, at least 125%, at least 130%, at least 135%, at least 140%, at least 145%, or at least 150% relative to a control or baseline. In some embodiments, glucose metabolism is improved by 10-100%, 20-100%, 30-100%, 40-100%, 50-100%, 10-75%, 20-75%, 30-100%, 50-75%, 10-50%, 20-50%, 30-50%, or 40-50%, relative to a control or baseline.

Glucose metabolism may also be defined as the magnitude of reduction in blood glucose in a given amount of time. In some embodiments, at least 5%, least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 105%, at least 110%, at least 115%, at least 120%, at least 125%, at least 130%, at least 135%, at least 140%, at least 145%, or at least 150% more glucose is metabolized in a given time, relative to a control or baseline. In some embodiments, 20-100%, 30-100%, 40-100%, 50-100%, 10-75%, 20-75%, 30-100%, 40-75%, 10-50%, 20-50%, 30-50%, or 40-50%, more glucose is metabolized in a given time, relative to a control or baseline.

In some embodiments, the ALK4 inhibitor, ALK5 inhibitor, ACVR2A inhibitor, ACVR2B inhibitor, and/or TGFβRII inhibitor are administered in an amount effective to reduce body fat content in the subject relative to a control (e.g., baseline, prior to administration of the inhibitors, or following administration of only one of the inhibitors). The control may be a subject that is not administered a composition, or is administered a composition that does not contain any agents that inhibit signaling through ALK4, ALK5, ACVR2A, ACVR2B, and/or TGFβRII (e.g. a composition containing only a pharmaceutically acceptable excipient). The baseline may be the body fat content of a subject as measured before administration of a composition. Body fat content, also known as body fat percentage, is the total mass of fat contained in a body divided by the total mass of the body and expressed as a proportion or percentage. Body fat content in a subject may be determined using any number of body fat measurement methods that are known in the art and apparent to one of ordinary skill in the art (e.g., skin calipers, bioelectrical impedance, hydrostatic weighing, dual-energy X-ray absorptiometry, air-displacement plethysmography, 3-dimensional body scanning, and magnetic resonance imaging) (Lemos et al. Curr Opin Endocrinol Diabetes Obes. (2017). 24(5):310-314). In some embodiments, body fat content is reduced by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50% relative to a control or baseline. In some embodiments, body fat content is reduced by 5-100%, 10-100%, 20-100%, 30-100%, 40-100%, 50-100%, 10-75%, 20-75%, 30-100%, 40-75%, 50-75%, 10-50%, 20-50%, 30-50%, or 40-50%, relative to a control or baseline.

### EXAMPLES

### Example 1

### Functional Redundancy of Type I and Type II Receptors in the Regulation of Skeletal Muscle Growth by Myostatin and Activin A

To determine the effect of targeting the two known type II receptors for MSTN specifically in skeletal muscle, mice carrying floxed alleles for both Acvr2 and Acvr2b were generated and these alleles were targeted using a transgene expressing Cre recombinase from a myosin light chain promoter/enhancer (Myl1-Cre), which is expressed by skeletal muscle fibers but not by satellite cells (23). Quantification of receptor mRNA levels in various muscles showed that expression was reduced significantly in each of the floxed lines, with the residual expression likely representing RNA made either by type I fibers or by non-muscle cells (data not shown). Significantly, in mice in which a given receptor was targeted in muscle, expression levels of each of the other receptors were relatively unchanged, indicating that there was not a compensatory upregulation of expression of any of the other receptors.

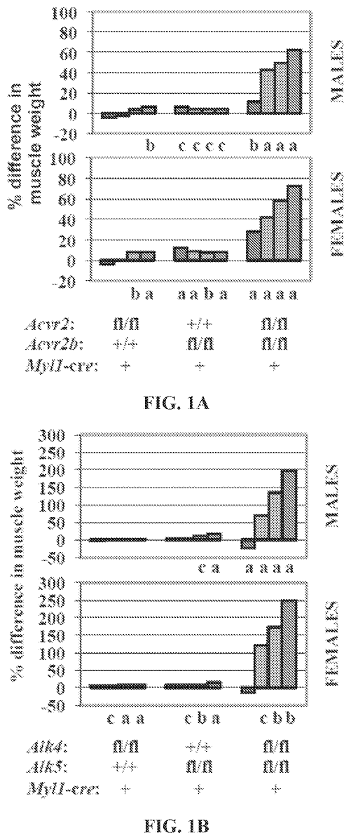

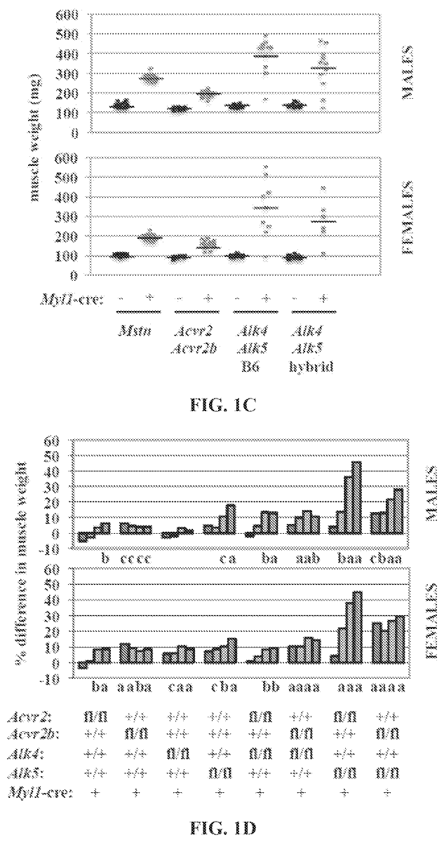

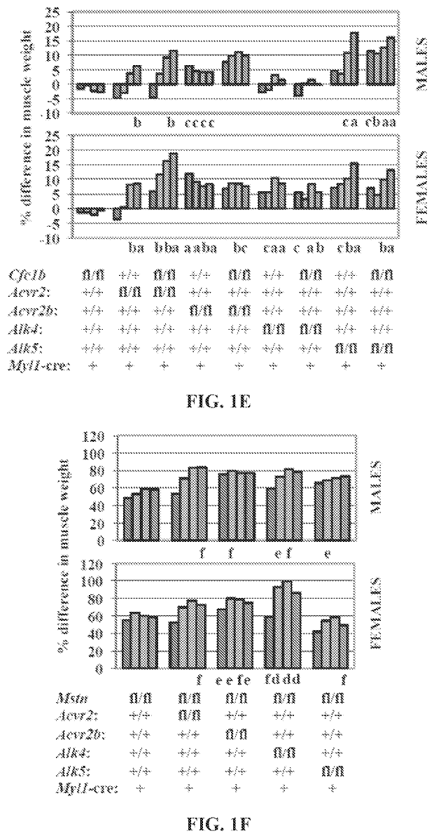

As reported previously, targeting Acvr2b alone in muscle fibers resulted in increases in muscle mass of about 8-12% in females and about 4-6% in males depending on the specific muscle examined (FIG. 1A). Targeting Acvr2 alone resulted in similar, statistically significant increases in the quadriceps and gastrocnemius but not in the pectoralis or triceps. Targeting both receptors simultaneously resulted in much more substantial increases in all muscles examined, with the greatest effect being seen in the quadriceps (58% and 50% in females and males, respectively) and gastrocnemius (72% and 62% in females and males, respectively). These results show definitively that Acvr2 and Acvr2b are functionally redundant with one another with respect to limiting muscle mass and that targeting signaling just in myofibers is sufficient to induce significant muscle growth, confirming the conclusions from earlier studies that myofibers are the primary direct target for signaling by MSTN in the regulation of muscle growth.

In vitro studies have suggested that binding of MSTN to ACVR2 and/or ACVR2B leads to engagement of the type I receptors ALK4 and ALK5. To provide genetic support for roles of these type I receptors in mediating MSTN signaling in vivo, the effect of targeting Alk4 and Alk5 specifically in myofibers was analyzed. For Alk4, a mouse line was generated in which exons 2-3 were flanked with LoxP sites (data not shown); removal of these exons by Cre recombinase would be predicted to delete the entire ligand binding and transmembrane domains and result in a null allele. For Alk5, a previously described floxed Alk5 line was used. As in the case of the type II receptors, the Myl1-Cre transgene significantly reduced RNA levels for both Alk4 and Alk5 in muscle (data not shown). Targeting Alk4 or Alk5 in myofibers resulted in statistically significant effects on muscle mass, ranging from up to 11% in the case of Alk4 and up to 18% in the case of Alk5 depending on the sex of the mice and the specific muscles examined (FIG. 1B). As in the case of the type II receptors, targeting both type I receptors simultaneously resulted in much more substantial increases, with the greatest effects being seen in the quadriceps (173% and 136% in females and males, respectively) and gastrocnemius (249% and 197% in females and males, respectively). These increases were even more pronounced than those seen in Mstn −/− mice and more reminiscent of the magnitude of effects seen upon targeting multiple ligands. These results demonstrate that both ALK4 and ALK5 play critical roles in limiting skeletal muscle mass in vivo, that these two receptors are functionally redundant in muscle, and that together, they regulate signaling by multiple ligands in myofibers, most likely MSTN and activin A.

The increases seen upon targeting the two type I receptors were much more extensive than those seen upon targeting the two type II receptors. This finding raised the possibility that another type II receptor may be utilized in addition to ACVR2 and ACVR2B. One possible candidate was BMPRII, although significant binding of MSTN to BMPRII was not detected in transfected cells. Moreover, a previous study suggested that BMP signaling may actually have the opposite effect of inducing muscle growth; specifically, this study reported that expression of a constitutively active type I BMP receptor (ALK3) can induce muscle growth and, conversely, that overexpression of the BMP inhibitor, noggin, can cause muscle atrophy not only in wild type mice but also in Mstn −/− mice. Nevertheless, mice carrying a floxed TGFβRII allele were used to examine the possibility that MSTN and/or activin A may signal through BMPRII in muscle. Mice in which TGβRII was targeted in myofibers generally had lower muscle weights than cre negative mice, with the effects being more pronounced in females (7-13% depending on the muscle). Mice in which all three type II receptors (TGFβRII, Acvr2, and Acvr2b) were targeted had muscle weights that were comparable to those seen in mice in which just Acvr2 and Acvr2b were targeted. These results imply that BMPRII activity in Acvr2/Acvr2b mutant mice is unlikely to explain the difference seen between Acvr2/Acvr2b targeted mice and Alk4/Alk5 targeted mice.

The effects of targeting Alk4 in combination with Alk5 were striking not only in terms of their magnitude but also in terms of the variability from mouse to mouse. As shown in FIG. 1C, the weight of the gastrocnemius muscle was relatively consistent in wild type and Mstn −/− mice as well as in mice in which both Acvr2 and Acvr2b were targeted in myofibers. In contrast, the weight of the gastrocnemius muscle was highly variable in mice targeting both Alk4 and Alk5 in myofibers, ranging from wild type levels in some mice to over 5 times wild type levels in other mice. This variability, which was also seen in the other muscles that were examined, was unlikely to be due to one or more genetic modifiers segregating independently in these crosses, as similar distributions of effects were observed both on a hybrid C57BL/6-129SvJ and on a pure C57BL/6 background. Clearly, further investigation of the basis for this variability may provide important insights into the control of muscle growth by this signaling pathway. Given that the results of these genetic studies clearly demonstrated roles for both type II receptors and both type I receptors in mediating muscle growth, the question of whether all four possible combinations of type II and type I receptors (ACVR2/ALK4, ACVR2/ALK5, ACVR2B/ALK4, and ACVR2B/ALK5) are utilized in vivo was investigated. These receptors were targeted in a pairwise manner, such that one type II receptor and one type I receptor would be targeted. The rationale for this approach was that by targeting just one receptor of each type, only a single possible type II/type I receptor combination would remain free to respond to myostatin. For example, by targeting both Acvr2b and Alk5, the role of ACVR2/ALK4 was examined, which is the only one of the four possible combinations whose function would be preserved in these mice. As shown in FIG. 1D, targeting both Acvr2b and Alk5 resulted generally in greater increases in muscle mass than targeting either alone, implying that the ACVR2/ALK4 combination cannot be the sole one that is used in vivo; however, the increases seen in these mice did not approach those seen either in mice lacking both type II receptors or in mice lacking both type I receptors, implying that the ACVR2/ALK4 combination does play some role in signaling. By targeting all four pairwise combinations in this manner, it was determined that all four type II/type I combinations are functional in vivo and that no one combination is sufficient to maintain wild type signaling levels in the absence of the other combinations. The most significant effects were observed in mice in which both Acvr2 and Alk5 were targeted, which exhibited muscle mass increases of approximately 40% in some muscles, implying that the ACVR2B/ALK4 combination is the least important of the four combinations in limiting muscle growth; these increases, however, were significantly less than those seen even upon targeting both Acvr2 and Acvr2b, implying that the ACVR2B/ALK4 combination is capable of playing some role in regulating growth of myofibers.

Another receptor component that has been implicated in MSTN signaling is Cripto (Cfc1b), which is known to serve as a co-receptor for certain ligands and to antagonize the activity of other ligands. In this regard, one study using C2C12 myoblasts reported that Cripto is required for MSTN signaling but inhibits activin A signaling; another study, however, showed that during muscle regeneration in vivo, Cripto expressed by satellite cells acts to antagonize MSTN signaling. To determine whether Cripto plays a role in regulating signaling in myofibers, the Myl1-Cre transgene was used to target Cfc1b either alone or in combination with each of the type I or type II receptors. As shown in FIG. 1E, no effect of targeting Cfc1b alone on muscle mass was observed. There was, however, an effect of targeting Cfc1b in combination with Acvr2, which resulted in small, but significant increases in muscle mass compared to targeting Acvr2 alone. This finding implies that at least some signaling through ACVR2B requires Cripto function, although the fact that the effects seen in these mice were substantially lower than those seen in mice in which both Acvr2 and Acvr2b were targeted implies that most signaling through ACVR2B in myofibers does not require Cripto. Significantly, no consistent effect of targeting Cfc1b in combination with Alk5 was observed, implying that Cripto expressed in myofibers is not required for signaling through ALK4 in myofibers, which contrasts with what has been reported in cell culture studies (30).

Previous studies showed that the function of MSTN in limiting muscle growth is redundant with that of at least one other TGF-β family member (13, 14), and several studies have implicated activin A as at least one of the key cooperating ligands (15, 16). To determine whether particular receptor components are used exclusively by particular ligands, the effects of targeting each of the type II and type I receptors in combination with Mstn were analyzed. Targeting Mstn specifically in myofibers utilizing mice carrying a previously generated floxed Mstn allele resulted in a reduction of Mstn mRNA in muscle by 95-99% and a reduction in circulating levels of MSTN protein by 92% and 85% in females and males, respectively (data not shown). It is presumed that the residual expression represents MSTN made by type I fibers, and although the possibility of non-muscle-derived MSTN contributing significantly to the circulating pool cannot be excluded, these data are consistent with myofibers being at least the predominant (if not sole) source of circulating MSTN protein.

Targeting Mstn in myofibers led to increases in muscle mass of 55-64% and 49-59% in females and males, respectively, depending on the specific muscle (FIG. IF). The approach used herein was to determine whether targeting a receptor component in addition to Mstn would result in further increases in muscle mass. The rationale was that by simultaneously targeting, for example, both Mstn and one type II (or type I) receptor, the contribution of the other type II (or type I) receptor in mediating activin A signaling could be determined. In general, targeting a given receptor in combination with Mstn led to greater increases in muscle mass compared to targeting Mstn alone. Of the tested combinations, the greatest statistically significant effects were seen in females in which Mstn was targeted along with Acvr2b or Alk4. These results imply that ACVR2B and ALK4 (and perhaps the combination of these two receptors) are likely to be utilized by activin A for signaling in myofibers more than ACVR2 and ALK5; however, these additional increases seen upon targeting a given receptor in combination with Mstn were still relatively small compared to the dramatic increases that observed in previous studies in which multiple ligands were targeted simultaneously, suggesting that the ACVR2B/ALK4 combination is unlikely to be the sole receptor combination that is utilized by activin A in myofibers in vivo.

The findings presented here as well as a prior study demonstrate that myofibers are the primary cellular targets for signaling by MSTN/activin A with respect to the regulation of muscle growth by these ligands. A variety of studies, however, have reported additional effects of genetic and pharmacological targeting of MSTN/activin A on other physiological processes. These findings have raised the question as to whether these other physiological effects reflect loss of MSTN/activin A signaling to other cell types and tissues or whether these are indirect effects resulting from inhibition of signaling to myofibers. In this respect, both MSTN and activin A are known to circulate in the blood, and these receptors are expressed by multiple cell types in multiple tissues. Because targeting receptors in myofibers resulted in significant effects on muscle growth, other physiological effects were investigated in these targeted mice to attempt to address this fundamental question. These studies focused on mice in which the two type II receptors, ACVR2 and ACVR2B, were targeted. Although the magnitude of the muscle mass increases was significantly larger in mice in which the two type I receptors were targeted, mice lacking ACVR2 and ACVR2B exhibited a much more consistent phenotype (as discussed above), making these studies easier to interpret.

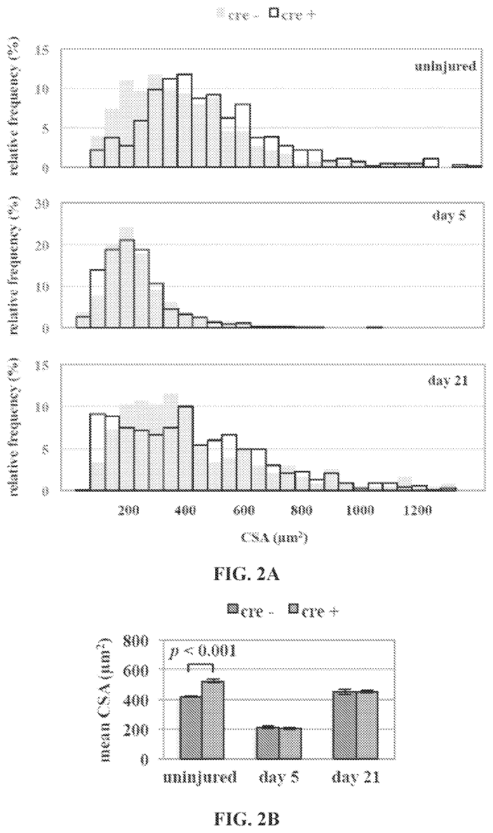

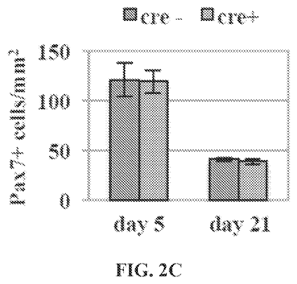

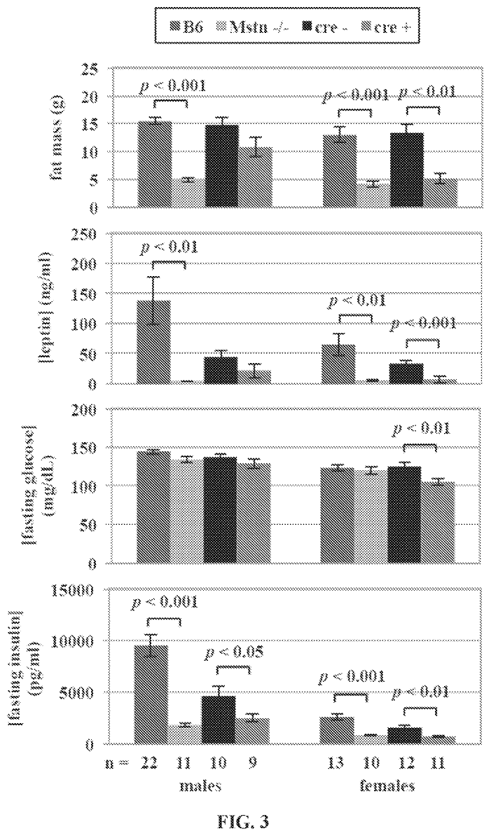

One set of studies examined the effect of targeting Acvr2 and Acvr2b in myofibers on muscle regeneration. Many studies have shown beneficial effects of targeting MSTN signaling on muscle regeneration in the setting of muscle degeneration or following muscle injury. A key question in this regard is whether these beneficial effects reflect inhibition of direct MSTN signaling to muscle satellite cells or whether inhibition of MSTN signaling to myofibers can enhance satellite cell activation and/or function in the setting of muscle degeneration or injury. To this end, muscle damage and regeneration were induced by injection of barium chloride intramuscularly in Acvr2 fl/fl-Acvr2b fl/fl mice carrying the Myl1-Cre transgene compared to mice lacking Myl1-Cre. The injured muscles were examined at 5 days and 21 days post-injury (DPI) to assess the effect of myofiber-specific knockout of Acvr2 and Acvr2b on muscle regeneration. As shown in FIGS. 2A-2B, fiber cross sectional area (CSA) was significantly greater in uninjured muscles lacking both Acvr2 and Acvr2b. In contrast, no differences in fiber CSA were observed in injured muscles assessed at 5 DPI and 21 DPI. Next, the effects of targeting Acvr2 and Acvr2b in myofibers on the expansion and self-renewal of satellite cells during the regenerative process were examined. No differences were observed in the number of Pax7⁺ cells, either at 5 DPI or 21 DPI, between Acvr2 fl/fl-Acvr2b fl/fl mice carrying the Myl1-Cre transgene compared to mice lacking Myl1-Cre (FIG. 2C). These results suggest that loss of Acvr2 and Acvr2b in the muscle fibers results in no significant alteration in satellite cell function during muscle regeneration.

Another physiological consequence of MSTN loss or inhibition in addition to stimulating muscle fiber growth is an overall effect on fat and glucose metabolism. Mstn −/− mice exhibit a significant suppression of fat accumulation and improved glucose metabolism in an otherwise wild type background as well as in ob/ob and agouti lethal yellow backgrounds. Beneficial metabolic effects have also been described in mice treated with MSTN inhibitors. A key question is whether these beneficial effects on fat accumulation and glucose metabolism are the result of inhibition of MSTN signaling to myofibers, leading to muscle growth, or whether they reflect lack of direct MSTN signaling to other cell types and tissues, including adipose tissue. Previous studies showed that differences in fat accumulation between Mstn −/− and wild type C57BL/6 mice become more pronounced as mice age. Hence, initial analysis was focused on one year old mice. By DXA analysis, total body fat content in one year old Mstn −/− mice was reduced to less than one-third that of wild type C57BL/6 mice, with a concomitant reduction in percent body fat (FIG. 3). Similarly, total fat content and percent body fat were reduced in Acvr2 fl/fl-Acyr2b fl/fl mice carrying the Myl1-Cre transgene compared to mice lacking Myl1-Cre, with the effects being more pronounced in females. Consistent with these differences in body fat content, serum leptin levels were also reduced in Mstn −/− mice as well as in Acvr2 fl/fl-Acvr2b fl/fl mice carrying the Myl1-Cre. Hence, the reduced fat mass seen upon loss of MSTN signaling is likely an indirect effect of increased muscling in these mice. Fasting glucose levels were generally similar among the different genotypes at this age, although statistically significant lower fasting glucose levels were observed in Acvr2 fl/fl-Acyr2b fl/fl female mice carrying the Myl1-Cre transgene compared to mice lacking Myl1-Cre. Most importantly, these normal to lower fasting glucose levels were maintained despite significantly lower fasting insulin levels both in Mstn −/− mice compared to wild type C57BL/6 mice and in Acvr2 fl/fl-Acvr2b fl/fl mice carrying the Myl1-Cre transgene compared to mice lacking Myl1-Cre with the effect again being more pronounced in females.

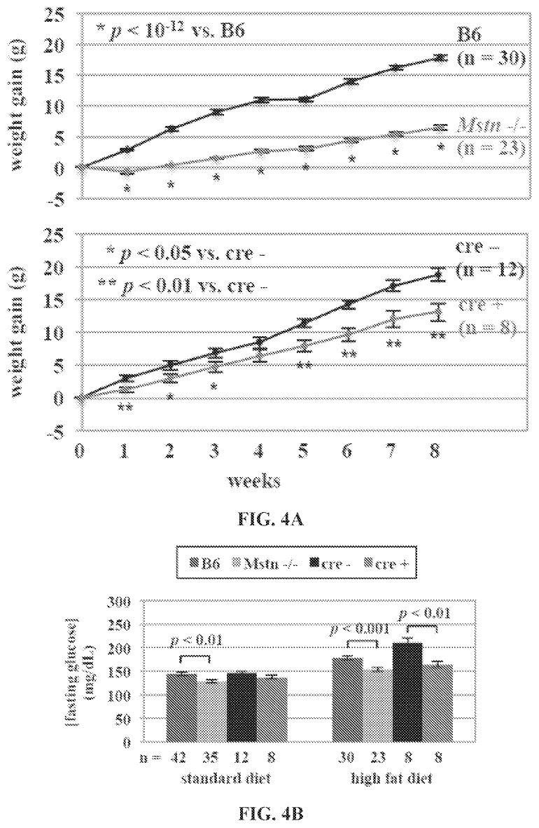

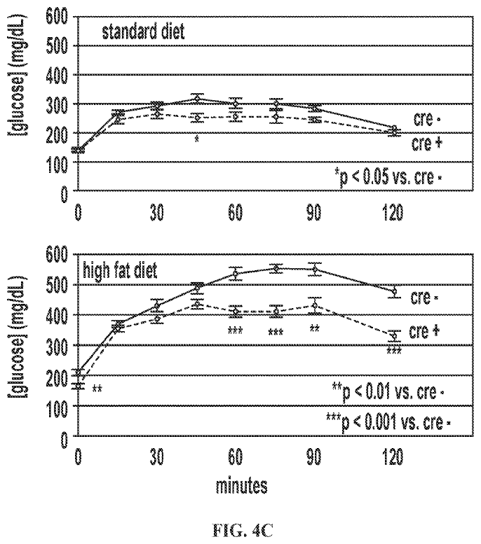

The effects of placing these mice on high fat diets were also observed. Younger mice (12 weeks of age) of both sexes were monitored in these studies. Although effects were generally similar in both males and females, they were more pronounced in males. As shown in FIG. 4A, Mstn −/− mice gained much less weight than wild type mice throughout an 8-week period on a high fat diet. Similarly, Acvr2 fl/fl-Acyr2b fl/fl mice carrying the Myl1-Cre transgene gained significantly less weight on a high fat diet than mice lacking Cre. These mice also differed in terms of glucose metabolism when maintained on a high fat diet. Fasting glucose levels were lower in Mstn −/− mice compared to wild type mice, and this lowering of fasting glucose levels was also seen in Acvr2 fl/fl-Acyr2b fl/fl-Myl1-Cre mice compared to mice lacking Cre (FIG. 4B). In glucose tolerance tests of mice maintained on standard diets, Acvr2 fl/fl-Acyr2b fl/fl-Myl1-Cre mice had glucose levels that trended lower, although only one time point reached statistical significance (FIG. 4C). Following 4 weeks on a high fat diet, however, these differences were accentuated, with Acvr2 fl/fl-Acyr2b fl/fl-Myl1-Cre mice having significantly lower glucose levels, particularly in the later time points. Taken together, these results clearly demonstrate that targeting this signaling pathway specifically in myofibers can lead to beneficial effects on fat and glucose metabolism.

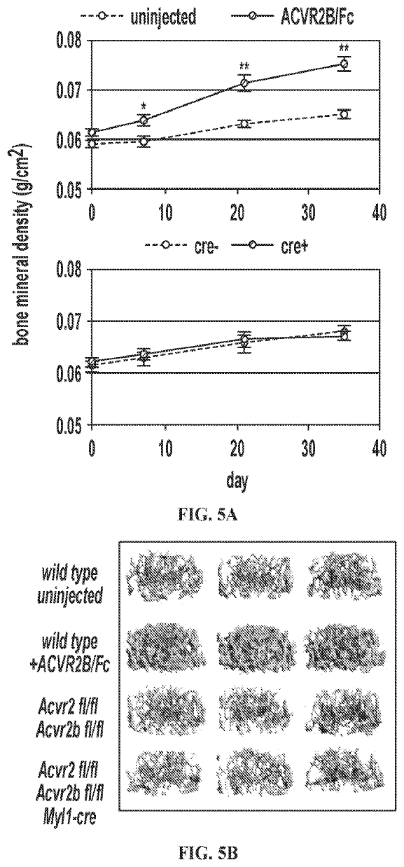

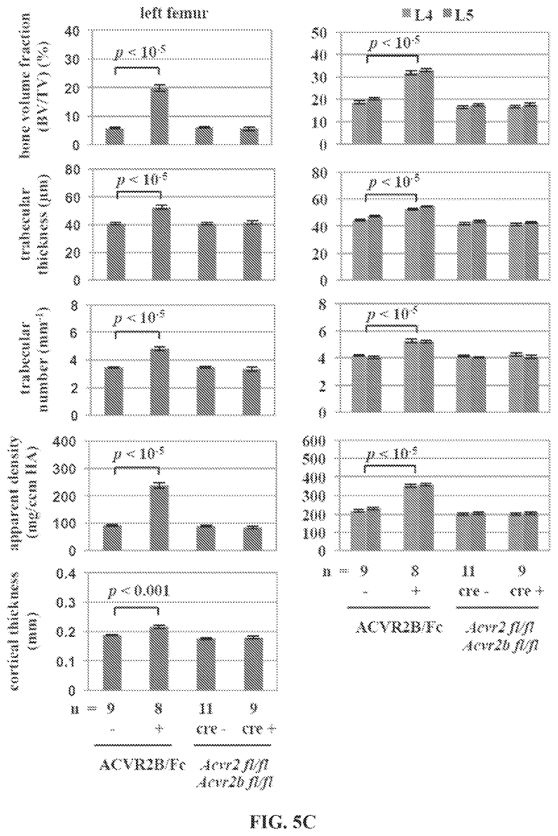

Finally, the effects of these targeted deletions on bones were examined. Prior studies have identified effects on bone density and structure in Mstn −/− mice. In addition, administration of a soluble form of the type IIB receptor (ACVR2B/Fc) systemically to mice can increase not only muscle mass, but also increase bone mineral density. At least part of this effect is due to inhibition of signaling directly to bone (likely by activin A), as targeting Acvr2 and Acvr2b in osteoblasts is sufficient to increase bone density in vivo. It is also possible, however, that some of the effects on bone may be indirect as a result of increased mechanical load on the bone due to enhanced muscle mass resulting from inhibition of signaling to myofibers. To test the role of increased muscle mass on bone, the bones of mice in which Acvr2 and Acvr2b were targeted in myofibers were compared to the bones of wild type mice, Cre-negative mice, and mice receiving the ACVR2B/Fc decoy receptor. Systemic administration of the ACVR2B/Fc decoy receptor to mice can induce rapid and significant muscle growth, and at the dose used in a previous study, individual muscle weights increased by about 40-50% over the 5-week treatment period (data not shown). Administration of the decoy receptor to wild type mice also resulted in significant increases in bone density as assessed by DXA analysis, with bone mineral density being approximately 15% higher in treated compared to untreated mice after 4 weeks of treatment with ACVR2B/Fc (FIG. 5A). This bone anabolic effect was confirmed by microCT analysis (FIGS. 5B-5C), which showed dramatic increases in bone volume, bone surface, and trabecular thickness and number in both the femur and L4 and L5 vertebrae in ACVR2B/Fc-treated mice. In contrast, bones of mice in which Acvr2 and Acvr2b were targeted in myofibers exhibited no statistically significant differences in any of these parameters. These results demonstrate that increasing muscle mass per se by targeting these receptors specifically in myofibers does not lead to corresponding increases in bone density and that the effect of ACVR2B/Fc in increasing bone density is almost certainly due to inhibition of direct signaling to bone, likely by activin A. Like other TGF-β family members, myostatin (MSTN) signals by utilizing a two-component receptor mechanism. Using biochemical approaches, it was previously shown that MSTN is capable of binding initially to either of the two type II receptors, ACVR2 (also called ACVR2A or ActRIIA) and ACVR2B (also called ActRIIB) Because MSTN appeared to bind ACVR2B with higher affinity than ACVR2 and because overexpression of a truncated form of ACVR2B (lacking the cytoplasmic kinase domain) in skeletal muscle could phenocopy the Mstn loss-of-function mutation in terms of increased muscle mass, it was presumed that ACVR2B is the primary type II receptor utilized by MSTN in vivo. In a follow-up genetic study, however, mice null for either Acvr2 or Acvr2b exhibit increased muscling and that these two receptors are partially functionally redundant. One limitation of that study was that examining the consequence of complete loss of both receptors was not possible using the deletion alleles for these genes because mice completely lacking both receptors are not viable. In order to circumvent this lethality issue, these receptors were targeted specifically in myofibers, as myofibers are direct targets for MSTN signaling and that blocking signaling in myofibers is sufficient to induce muscle growth. These receptors were targeted by crossing in a myosin light chain-Cre (Myl1-Cre) transgene, which is expressed specifically by myofibers, into mice carrying floxed alleles for Acvr2 and Acvr2b. Targeting Acvr2 and Acvr2b individually resulted in small, though significant, increases in muscle mass but that simultaneously targeting both type II receptors leads to much more dramatic effects, demonstrating conclusively that both receptors function to suppress muscle growth and that the two receptors are functionally redundant in this regard.

The identification of ACVR2 and ACVR2B as MSTN receptors led to two strategies to develop therapeutics targeting MSTN signaling to treat patients with muscle loss or degeneration. One approach was to generate a soluble form of ACVR2B in which the ligand-binding domain was fused to an immunoglobulin Fc domain. Indeed, this decoy receptor (ACVR2B/Fc) is still the most potent agent described to date in terms of its ability to promote muscle growth; in fact, just two injections of this decoy receptor at high doses to mice can cause greater than 50% muscle growth throughout the body in just two weeks. Although this effect of the decoy receptor has been widely cited in the literature as evidence that ACVR2B is the primary receptor utilized by MSTN in vivo, it is important to note that because this decoy receptor is a ligand trap, it is capable of blocking signaling of ligands through other receptors as well. A second approach, which was taken by Novartis, was to develop a monoclonal antibody targeting ACVR2B directly. Although the initial publication of this monoclonal antibody (bimagrumab, or BYM338) reported that it had over 200-fold higher affinity for ACVR2B compared to ACVR2, subsequent studies showed that by x-ray crystallography analysis, BYM338 is capable of blocking the ligand binding domain of both receptors. They further showed that highly specific monoclonal antibodies directed against each receptor (with no cross-reactivity to the other receptor) had an additive effect in terms of stimulating muscle growth when given to mice. Hence, these pharmacologic studies taken together with genetic studies presented here demonstrate conclusively that both ACVR2 and ACVR2B play critical roles in regulating muscle growth.

Binding of MSTN to the type II receptors then leads to engagement of the two type I receptors, ALK4 and ALK5. Although a number of studies have examined the roles of these receptors in cell culture systems, very few studies have addressed the functions of these receptors in vivo with respect to control of muscle growth. Some of these in vivo studies reached somewhat contradictory conclusions. In particular, whereas small molecule inhibitors of ALK4/5 were shown to induce muscle fiber hypertrophy in both wild type and dystrophic mice as well as to preserve muscle mass in a cancer cachexia model, delivery of an antisense oligonucleotide directed against Alk4 had the opposite effect, leading to a reduction in muscle mass. Here, a genetic approach used the Myl1-Cre transgene to target floxed Alk4 and Alk5 alleles in myofibers. Targeting Alk4 alone had no effect on muscle mass and that targeting Alk5 alone has a small, though significant effect. Targeting both type I receptors simultaneously, however, led to dramatic increases in muscle size. These increases were substantially higher than those seen upon targeting ACVR2 and ACVR2B, raising the possibility that yet another type II receptor may also be involved in regulating muscle mass. Targeting TGFBRII along with ACVR2 and ACVR2B did not cause further increases in muscle mass, leaving TGFBR2 as the only remaining candidate, although no binding of either MSTN or activin A to TGFBR2 has been reported.

These increases seen upon targeting both Alk4 and Alk5 even surpassed the doubling of muscle mass seen in mice completely lacking MSTN and are more reminiscent of the quadrupling of muscle mass seen in Mstn −/− mice that also carry a transgene overexpres sing the MSTN inhibitor, follistatin, in skeletal muscle. The ability of follistatin as well as the ACVR2B/Fc decoy receptor to induce further muscle growth even in Mstn −/− mice showed that at least one other TGF-β family member besides MSTN must also play an important role in suppressing muscle growth, and subsequent studies have identified activin A as the key cooperating ligand. The dramatic increases in muscle growth that were obtained by targeting both Alk4 and Alk5 in myofibers suggests that these receptors must be involved in mediating signaling by both MSTN and activin A to myofibers. Furthermore, by simultaneously targeting different combinations of type I and type II receptors and by simultaneously targeting Mstn with each type I and type II receptor, it was possible to investigate whether specific receptor combinations are utilized by specific ligands. Although some quantitative differences were detected by targeting different combinations, the overall conclusion from all of these studies is that muscle mass is regulated by at least two ligands (MSTN, activin A) signaling directly to myofibers through two type II (ACVR2, ACVR2B) and two type I (ALK4, ALK5) receptors, with all ligand and receptor combinations being utilized in a highly redundant manner. These findings suggest that in order to generate maximal effects on muscle growth, it is essential that any therapeutic strategy be capable of targeting either multiple receptor components simultaneously, such as with BYM338, or multiple ligands simultaneously, such as with the ACVR2B/Fc decoy receptor.

These studies also demonstrate conclusively that myofibers are the direct targets for MSTN and activin A signaling with respect to control of muscle growth. Given that it was possible to generate substantial effects on muscle mass by blocking MSTN/activin signaling just in myofbers, this experimental approach was used to investigate other physiological effects in these mice. In particular, previous studies using Mstn −/− mice have identified effects of MSTN loss on other tissues besides skeletal muscle, raising the question as to whether MSTN is capable of signaling directly to these other tissues in vivo or whether these are indirect effects of lack of MSTN signaling to skeletal muscle. In the studies presented here, direct versus indirect effects were distinguished by blocking MSTN and activin A signaling in just myofibers. Although targeting the two type I receptors generated the greatest effects on muscle mass, the phenotype was highly variable in these mice, and further experiments therefore focused on mice in which the two type II receptors, ACVR2 and ACVR2B, were targeted, as the phenotype was much more consistent.

One tissue other than skeletal muscle known to be affected in Mstn −/− mice is adipose tissue. Mstn −/− mice have a reduction in fat accumulation, particularly as a function of age, not only in a wild type background but also in ob/ob and agouti lethal yellow backgrounds, as well as beneficial effects on glucose metabolism. Furthermore, loss or inhibition of MSTN can increase skeletal muscle glucose uptake and energy expenditure and protect against high fat diet-induced weight gain as well as glucose intolerance. A key question is whether each effect on adipose tissue and glucose metabolism reflects a loss of MSTN signaling to skeletal muscle, or some effects reflect a loss of MSTN signaling to other tissues. In this regard, a previous study showed that mice overexpressing a truncated form of ACVR2B in skeletal muscle also exhibit some of the metabolic effects seen in Mstn −/− mice. However, because this truncated receptor could act as a ligand trap, one possibility is that one mode of action of this truncated receptor may be to act as a sink by binding MSTN produced by skeletal muscle and thereby leading to inhibition of MSTN signaling not only to muscle but also to other tissues. Indeed, among the cell types known to be responsive to MSTN in cell culture are adipocytes. Moreover, although Mstn is expressed at low levels in adipose tissue in wild type mice, Mstn expression is significantly upregulated in both subcutaneous and visceral fat in ob/ob mice. To address whether the effects on adipose tissue and glucose metabolism seen in Mstn −/− mice reflect direct or indirect effects of MSTN loss, the metabolic effects of targeting Acvr2 and Acvr2b in myofibers were analyzed. These mice, like Mstn −/− mice, had reduced overall body fat, lower serum leptin levels, and reduced weight gain on a high fat diet. These receptor-targeted mice also had lower fasting blood glucose despite having lower fasting insulin levels and are able to maintain lower glucose levels in glucose tolerance tests. These findings demonstrate that these metabolic effects as well as the suppression of fat accumulation can be achieved by inhibition of signaling solely in myofibers.

Another tissue known to be affected in Mstn −/− mice is bone. In particular, Mstn −/− mice have been reported to have a generalized increase in bone mineral density at many sites, including femurs. A key question raised by these findings is whether this increased bone mineral density results from increased mechanical load on the bones due to the hypermuscularity in these mice or rather from loss of MSTN signaling directly to bone. In this regard, MSTN has been reported to be capable to acting directly on bone progenitor cells in vitro to regulate cell differentiation. It is also known that MSTN inhibitors, like follistatin and the ACVR2B/Fc decoy receptor, can have significant effects on bone repair and bone density in vivo, but because these inhibitors can also block activin signaling, the identities of the key ligands being blocked in these studies is not clear. Similarly, it is known that targeting type II receptors in osteoblasts in vivo can also increase bone density, but this effect likely reflects inhibition of signaling by activin rather than by MSTN. In order to determine the contribution of increased skeletal muscle mass on bone density, the bones of mice in which Acvr2 and Acvr2b were in myofibers were analyzed. Whereas systemic administration of the ACVR2B/Fc decoy receptor to wild type mice can induce dramatic increases in both muscle mass and bone mineral density, increasing muscle mass by targeting these receptors in myofibers had no effect on bone mineral density either in the femurs or in the lumbar vertebrae. These findings demonstrate that the effect of the ACVR2B/Fc decoy receptor on increasing bone mass is almost certainly due to inhibition of signaling by TGF-β-related ligands directly to bone and, furthermore, that increasing muscle mass by up to even 60-70% in the hindlimbs was not sufficient to increase bone density either through increased mechanical load on the bones or through the release of hypothetical secondary mediators by muscle.

Finally, blocking signaling specifically in myofibers also had no effect on muscle regeneration, implying that the effects of MSTN loss or inhibition observed in prior studies likely reflect inhibition of signaling directly to satellite cells. Although a formal demonstration of the role of signaling in satellite cells will require targeting these receptors specifically in that cell population, these findings suggest that MSTN and/or activin A signal to multiple cell types within skeletal muscle and play at least two distinct roles in regulating muscle homeostasis, one to regulate myofiber growth and another to regulate muscle regeneration following injury.

### Materials and Methods

All animal experiments were carried out in accordance with protocols that were approved by the Institutional Animal Care and Use Committees at the Jackson Laboratory, University of Connecticut School of Medicine, and Johns Hopkins University School of Medicine. Mice carrying floxed alleles for Acvr2, Acvr2b, Alk5, TGFβRII, Cfc1b, and Mstn have been described previously. To generate Alk4 conditional knockout mice, a targeting construct was generated in which exons 2-3 were flanked with LoxP sites (not shown). Following electroporation of the targeting construct into embryonic stem (ES) cells, ES cell colonies carrying the homologously-targeted allele were injected into blastocysts, and mice generated from these blastocysts were bred to identify those exhibiting germ-line transmission of the targeted allele. Offspring from these matings were then bred with EIIa-Cre transgenic mice in order to delete the neomycin resistance cassette in the germ-line. From these crosses, mice carrying an Alk4 flox allele lacking the NEO cassette were obtained.

For measurement of muscle weights, individual muscles were dissected from both sides of 10-week-old mice, and the average weight was used for each muscle. Circulating MSTN levels were determined on acid activated serum samples by ELISA using the R&D Systems DGDF80 kit. To induce muscle damage and regeneration, 50μL 1.2% barium chloride (w/v) was delivered to the right TA muscle over ten intramuscular punctures. The left TA served as the uninjured control. TA muscles were harvested either 5 days or 21 days post injury, mounted in OCT, and frozen in thawing isopentane. Serial sections (8 μm) were cut transversely through the belly of the TA muscle using a refrigerated cryostat. TA sections were immunoreacted to laminin and Pax7 applied with the M.O.M Basic Kit. Sections were then counterstained with DAPI to visualize nuclei and imaged with a Zeiss Observer Z1 microscope with a color camera controlled by Volocity software. Images were then quantified using ImageJ software.

Live animal imaging was performed using a Piximus dual-energy X-ray absorptiometer (DXA). Glucose tolerance tests (GTT) were performed by giving mice an intraperitoneal injection of 1 g glucose/kg body weight following a 6 hour fast. Mice were then placed on a 60 kcal % fat diet (D12492) for 8 weeks, with a repeat GTT being performed after 4 weeks. The ACVR2B/Fc decoy receptor was expressed in Chinese hamster ovary cells, purified from the conditioned medium using a protein A Sepharose column, and administered intraperitoneally at a dose of 175 μg per injection. For microCT analysis, the left femur and lumbar vertebrae were placed in 70% ethanol. μCT was performed in a Scanco μCT40 at 8 μm3 resolution. Samples were scanned in 70% ethanol 55kVp, 145 μA intensity, 300 ms. The instrument is calibrated weekly using Scanco phantoms, and all scans passed routine quality control verification. Analysis of femurs and vertebrae was conducted using standard protocols, with a lower threshold of 2485 Hounsfield units (HU) for femoral trabeculae, 4932HU for femoral cortex, and 3078HU for vertebral trabeculae. Surface renderings were generated corresponding to each of these thresholds.

### Example 2

### Local Versus Systemic Control of Bone and Skeletal Muscle Mass by Components of the Transforming Growth Factor-β Signaling Pathway

Myostatin (MSTN) is a transforming growth factor-β (TGF-β) superfamily member that normally acts to limit skeletal muscle mass (1). Mice lacking MSTN exhibit dramatic increases in skeletal muscle mass throughout the body, with individual muscles growing to about twice the normal size. The amino acid sequence of MSTN has been strongly conserved through evolution (2) and engineered or naturally-occurring mutations in the MSTN gene have been shown to lead to increased muscling in many other species as well, including cattle (2-4), sheep (5), dogs (6), rabbits (7), rats (8), swine (9), goats (10), and humans (11). MSTN activity is regulated by various extracellular binding proteins, including follistatin (FST) (12), FSTL-3 (13), GASP-1 (14), and GASP-2 (15, 16) as well as the MSTN propeptide, which maintains MSTN in an inactive, latent state (12, 17-19). MSTN signals initially by binding to the activin type 2 receptors, ACVR2 and ACVR2B (12, 20-22), followed by engagement of the type 1 receptors, ALK4 and ALK5 (22, 23).

The function of MSTN as a negative regulator of muscle growth is partially redundant with that of another TGF-β family member, activin A (20, 24-27), which shares many regulatory and signaling components with MSTN. Indeed, one of these components, FST, was originally identified for its ability to inhibit secretion of follicle stimulating hormone (FSH) by cultured pituitary cells (28), and subsequent work showed that FST is capable of binding and inhibiting activins (29), which are capable of signaling to pituitary gonadotrophs to induce FSH secretion (30). FST undergoes alternative splicing to generate two isoforms, the full-length FST315 and a carboxyl-terminal truncated FST288 (31). A third form, FST303, is derived from proteolytic cleavage of the C-terminal domain. All of the FST isoforms contain a heparin binding domain that mediates binding to cell surface proteoglycans. The presence of the C-terminal acidic tail in FST315, however, appears to neutralize the basic residues present in the heparin binding domain, and as a result, FST315 binds poorly to proteoglycans and is the predominant form of FST in the circulation. FST288, which lacks the C-terminal 26 amino acid extension, tends to remain locally sequestered following secretion.

Based on the existence of these multiple isoforms and their differential biodistribution following secretion, a major question has been whether the mode of action of FST is primarily local, regulating signaling by target ligands at or near the site of FST synthesis, or whether circulating FST can influence signaling to tissues distant from its site of synthesis. This question is relevant not only in terms of understanding the mechanism of action of FST but also in terms of interpreting human studies seeking associations between circulating FST levels and various physiological and pathological states. Here, genetic approaches in mice were used to address this fundamental question with respect to the roles of FST and this regulatory system in regulating two tissues, skeletal muscle and bone.

A number of studies have shown that FST, by binding and inhibiting both MSTN and activin A, plays an important role in regulating muscle growth. Specifically, transgenic overexpression of FST in skeletal muscle leads to muscle hypertrophy, consistent with inhibition of the MSTN/activin A signaling pathway (12), and conversely, heterozygous loss of Fst in mice leads not only to reductions in muscle weights (by about 15-20%), but also to a shift toward oxidative fiber types, an impairment of muscle regeneration following cardiotoxin-induced injury, and reduced tetanic force production (25), all consistent with overactivity of this signaling pathway. Fst^{-/-} mice have also been shown to have a reduced amount of muscle at birth (32), but because mice completely lacking FST are not viable, mice carrying a conditional Fst^{flox} allele (33) were used to target Fst in specific cell types and regions of the body in order to examine the effects of FST loss in tissues of adult mice. Even after extensive backcrossing of the flox allele onto a C57BL/6 background, mice carrying this allele (in the absence of cre recombinase) were heavier than wild-type mice, with total body weights of Fst^{flox/flox} mice being increased by 13% and 19% in males and females, respectively. These differences in body weights reflected increased expression of Fst from the flox allele, likely resulting from retention of the neomycin selection cassette in the targeted locus during the construction of this mutant line (33).

The increased expression of Fst from the flox allele allowed for the generation of mice carrying various combinations of wild-type, deletion, and flox alleles to produce an allelic series with varying levels of Fst expression. Analysis of the gastrocnemius muscle showed that Fst RNA expression levels ranged from a 30% decrease in Fst ^{+/-} mice to 55% and 82% increases in Fst^{flox/+} and Fst^{flox/flox} mice, respectively (FIG. 5A). Levels of circulating FST also generally followed the same trends, with serum FST levels being approximately 50% and 200% of wild-type levels in Fst^{+/-} and Fst^{flox/flox} mice, respectively (FIG. 5B). These differences in Fst expression levels correlated not only with total body weight (Table S1) but also with weights of individual muscles, which ranged from decreases of 18-23% in Fst^{+/-} mice to increases of 29-48% in Fst^{flox/flox} mice depending on the specific muscle and sex (FIGS. 5A, 5C). Fst^{flox/+} and Fst^{flox/-} mice had intermediate muscle weights reflecting intermediate Fst expression levels in these mice. Hence, FST acts in a dose-dependent manner to regulate muscle mass, with an approximately linear relationship between levels of FST expression and muscle weights.

Because Fst^{-/-} mice die immediately after birth (32), the effect of complete loss of FST could not be examined in adult mice. To determine the effect of further loss of FST beyond that seen in Fst^{+/-} mice, the effect of targeting Fst^{flox} alleles in specific tissues and regions of the body was examined. In one set of experiments, the effect of targeting Fst in skeletal muscle was examined using an Myl1-cre transgene (34), which was previously shown to be expressed specifically in myofibers, but not in satellite cells (35). Male mice carrying one or more Fst^{flox} alleles in conjunction with Myl1-cre generally had lower circulating FST levels compared to mice lacking Myl1-cre, demonstrating that myofiber-derived FST does contribute to the circulating pool (FIG. 5B); similar trends were seen in female mice, although the individual comparisons were not statistically significant. Targeting Fst in myofibers resulted in decreases in muscle weights in both males and females; although the effects were relatively small, many of the differences were statistically significant in both sexes (FIG. 5C). Hence, myofiber-derived FST does play some role in regulating muscle mass.

Because the effects of targeting Fst in myofibers were small, however, the effects of more global targeting of Fst were also examined using the Cdx2-cre transgene, which is expressed specifically in the posterior but not anterior region of the animal (36); in particular, Cdx2-cre is expressed in all cells posterior to the umbilicus but not in any cells anterior to the umbilicus. In order to eliminate as much Fst expression as possible, Fst^{+/-}; Cdx2-cre males were crossed with Fst^{flox/flox} females, and Fst^{flox/-} offspring that were either negative or positive for Cdx2-cre were analyzed. Fst^{flox/-}; Cdx2-cre mice were viable, which allowed for analysis of mice at adulthood. As shown in FIG. 5D, muscles located in the posterior region of Fst^{flox/-}; Cdx2-cre mice (quadriceps and gastrocenemius), where Cdx2-cre is expressed, exhibited significant decreases in muscle weights that exceeded those seen in Fst^{+/-} mice (25; see also FIG. 5C). These decreases were seen in both males and females and ranged from 51-58% in both muscle groups compared to Fst^{flox/-} mice lacking Cdx2-cre.

To determine whether the decreased muscle sizes were due to differences in fiber numbers or fiber sizes, morphometric analysis of gastrocnemius muscles was conducted. Although no significant differences in fiber number between cre negative and cre positive muscles were observed, myofiber sizes were significantly reduced in cre positive muscles. As shown in FIG. 5E, the distribution of fiber sizes of Fst^{flox/-}; Cdx2-cre muscles had a similar overall shape to that of cre negative mice but was shifted significantly to smaller diameters, with the mean fiber diameter being reduced from 45.4 μm in Cdx2-cre negative mice to 33.6 μm in Cdx2-cre positive mice. For comparison, morphometric analysis of F66 transgenic mice, which exhibit dramatic increases in muscle mass as a result of an Myl1-Fst transgene located on the Y chromosome (24), was conducted. As reported previously, fiber number in the gastrocnemius was increased by 12% in F66 mice compared to wild-type mice, and the distribution of fiber diameters, which was slightly more spread out than in wild-type mice, was shifted to larger diameters, with mean fiber diameter being increased from 42.5 μm in wild-type mice to 59.7 μm in F66 mice (FIG. 5E). Hence, by manipulating levels of Fst expression, a 4-fold range in muscle size was generated, from a slightly over 50% decrease in Fst^{flox/-}; Cdx2-cre mice to an approximate doubling in F66 mice.

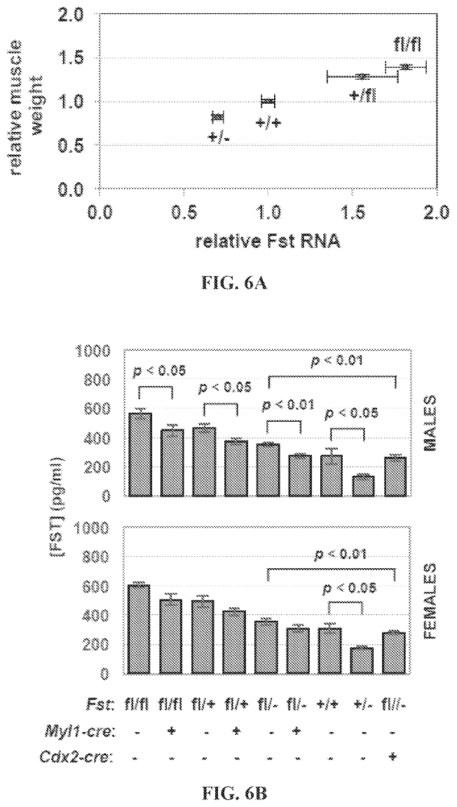

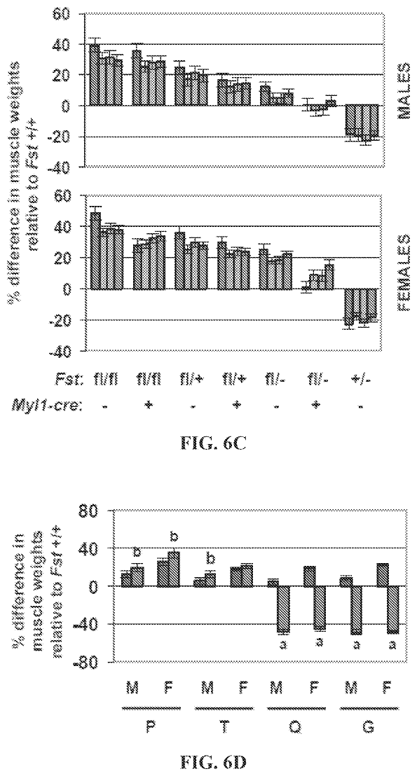

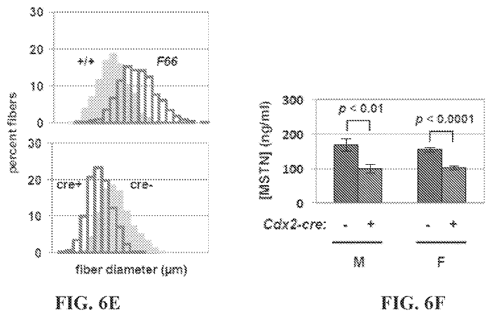

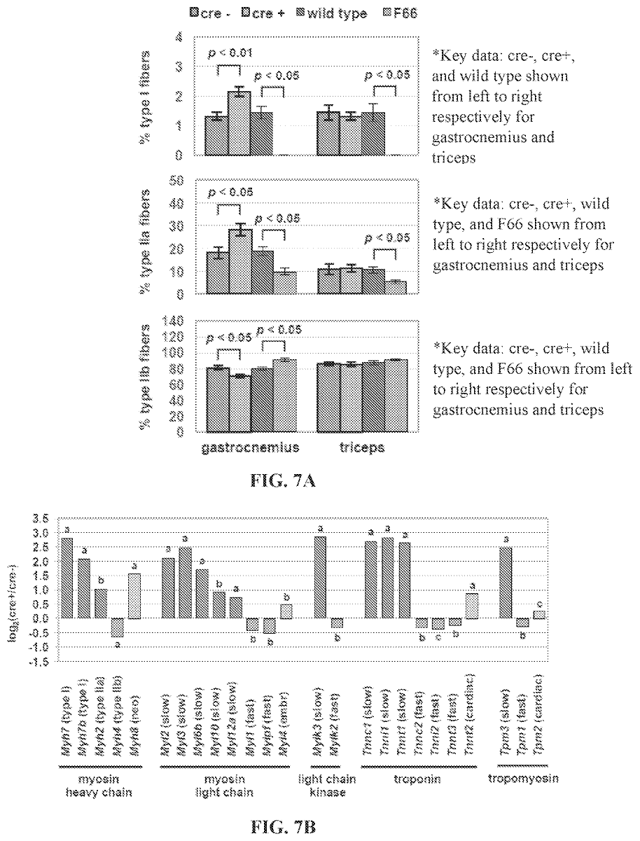

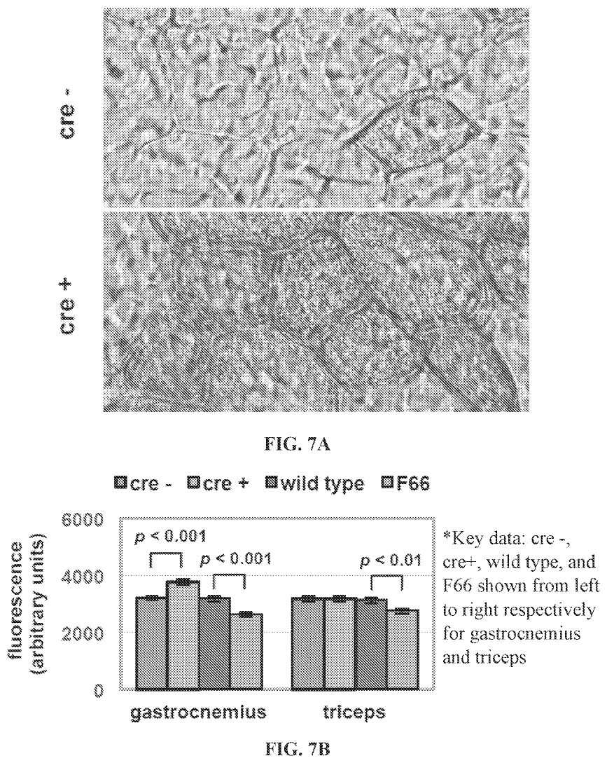

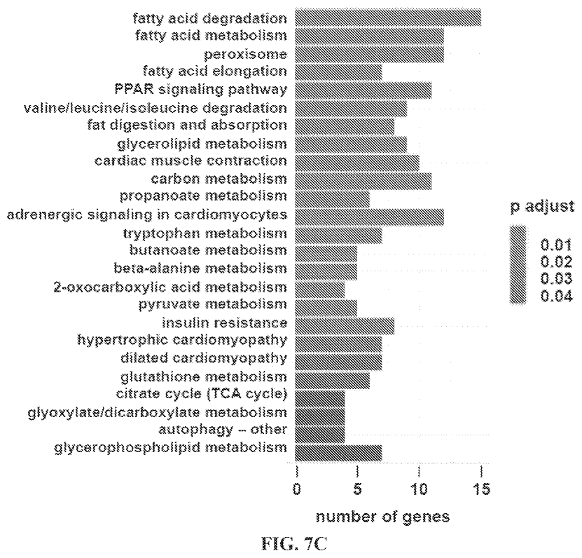

Further histological examination of muscles from Fst^{flox/-}; Cdx2-cre mice revealed two other effects of FST loss. First, fiber type analysis of gastrocnemius muscles revealed a significant shift away from glycolytic type 2b fibers and toward mixed glycolytic/oxidative type 2a fibers and oxidative type 1 fibers in Cdx2-cre positive muscles compared to cre negative muscles (FIG. 6A). This fiber type shift is consistent with that seen in Fst^{+/-} mice (25) but to a greater degree. In contrast, the reverse fiber type shift was seen in F66 muscles compared to wild-type muscles; in fact, in F66 gastrocnemius muscles, type 1 fibers were completely absent, and the percentage of type 2a fibers was reduced by nearly 50%. Hence, decreasing FST levels led to a fiber type shift toward more oxidative fibers, and increasing FST levels led to a fiber type shift toward more glycolytic fibers. Second, Oil Red O staining of gastrocnemius sections revealed the presence of numerous fat droplets in muscle fibers of Fst^{flox/-}; Cdx2-cre mice compared to cre negative controls (FIG. 7A). These fat droplets were seen in the majority of muscle fibers and not just in type 1 and type 2a fibers, which have been reported to show lipid accumulation by histological examination (37). Quantification of representative sections by fluorescence imaging of Nile red-stained sections showed significantly increased fluorescence intensity in gastrocnemius muscles isolated from Cdx2-cre positive compared to cre negative mice (FIG. 7B). We also analyzed sections of muscles isolated from F66 transgenic mice, which showed decreased fluorescence intensity compared to wild-type mice. Hence, the extent of fat accumulation in muscle was inversely correlated with levels of FST.

These histological changes with respect to fiber type shifts and lipid accumulation were consistent with changes in gene expression patterns in muscles isolated from these mice. RNA-seq analysis identified 399 up-regulated and 234 down-regulated transcripts in gastrocnemius muscles isolated from Cdx2-cre positive compared to cre negative mice (FIG. 11). Among these differentially regulated genes were ones encoding myosin heavy chain isoforms characteristic of specific fiber types (38, 39). In particular, Myh7, Myh7B, and Myh2 were all up-regulated in Cdx2-cre positive muscles, consistent with increased numbers of type 1 and type 2a fibers, and Mhy4 was down-regulated, consistent with decreased numbers of type 2b fibers (FIG. 6B). We observed similar shifts in other sarcomere components as well (38, 39), including myosin light chain, troponin, and tropomyosin isoforms, with components characteristic of slow fibers (Myl2, Myl3, Myl6b, Myl10, Myl12a, Tnnc1, Tnni1, Tnnt1, and Tpm3) being up-regulated and components characteristic of fast fibers (Myl1, Mylpf, Tnnc2, Tnni2, Tnnt3, and Tpm1) being down-regulated in Cdx2-cre positive muscles. Moreover, expression levels of myosin light chain kinase isoforms also tracked with these fiber type shifts, with Mylk3 (slow fibers) and Mylk2 (fast fibers) being up-regulated and down-regulated, respectively. Finally, up-regulation of certain sarcomere protein isoforms not typically expressed in adult skeletal muscle, including Myh8 (neonatal), Myl4 (embryonic), Tnnt2 (cardiac), and Tpm2 (cardiac) were observed, raising the possibility that there might be enhanced regeneration occurring in Cdx2-cre positive muscles. However, no increase was observed in the number of centrally-located nuclei in muscles of Cdx2-cre positive mice. Also consistent with the histological changes were the results of pathway analysis of the entire set of differentially-expressed transcripts in Cdx2-cre positive muscles. Although no enriched pathways were identified among the 234 down-regulated transcripts, 25 enriched pathways were identified among the 399 up-regulated transcripts. Strikingly, 7 of the top 8 enriched pathways with respect to statistical significance corresponded to pathways involved in lipid metabolism (FIG. 7C). These pathways encompassed a total of 40 up-regulated genes, 36 of which encode enzymes directly involved in lipid metabolism (Table 2). Hence, changes in RNA expression profiles in Cdx2-cre positive muscles were consistent with changes observed on histological examination of the muscles.

A considerably larger number of differentially expressed genes were identified in RNA-seq analysis of F66 muscles, with 2275 up-regulated and 2667 down-regulated transcripts compared to wild-type muscles (FIG. 11). Among the down-regulated transcripts in F66 muscles were 75 that were oppositely regulated (i.e. up-regulated) in Cdx2-cre positive mice. Pathway analysis of this subset of 75 genes identified three enriched pathways, namely thermogenesis, TCA cycle, and oxidative phosphorylation. These three enriched pathways, which comprised overlapping sets of genes, included a total of 17 genes whose functions were consistent with the shift toward oxidative fibers seen in Cdx2-cre positive mice and shift toward glycolytic fibers seen in F66 mice (Table 3). Among the up-regulated transcripts in F66 muscles were 101 that were oppositely regulated (down-regulated) in Cdx2-cre positive mice. Pathway analysis of this subset of 101 genes identified seven enriched pathways, encompassing an overlapping set of 38 genes. Among these 38 genes were Rps6kb1 encoding ribosomal protein S6 kinase B1, which plays an important role in regulating protein synthesis, three proto-oncogenes (Braf, Mras, Nras), and genes encoding 13 components of the cytoskeleton and extracellular matrix, including ankyrin 1, radixin, sarcoglycan alpha, decorin, thrombospondin 1, two integrin subunits, and six collagen chains (Table S3).

The dramatic effects of FST loss seen in the quadriceps and gastrocnemius of Fst^{flox/-}; Cdx2-cre mice clearly show the important role that FST plays in regulating muscle size and structure. The effects seen in these mice could result either from loss of local action by FST or from loss of systemic action by FST, or both. Indeed, circulating levels of FST were decreased by 26% and 21% in Fst^{flox/-}; Cdx2-cre males and females, respectively (FIG. 5B), reflecting the fact that FST production is eliminated in the posterior region of the body in these mice. Given that the phenotype of Fst^{flox/-}; Cdx2-cre mice is much more pronounced than that seen in Fst^{+/-} mice, which exhibit decreases in circulating FST levels by about 50%, and in Fst^{flox/-}; Myl1-cre mice, which exhibit decreases of slightly more than 20%, it seemed unlikely that systemic loss of FST could explain the greater effects seen in the quadriceps and gastrocnemius muscles of Fst^{flox/-}; Cdx2-cre mice compared to these other mice. To investigate further this question of local versus systemic modes of FST action, muscles located in the anterior region of the animal, where Cdx2-cre is not expressed, were analyzed. In contrast to the dramatic decreases in muscle weights seen in the posterior muscles, weights of anterior muscles (pectoralis and triceps) were not decreased in Fst^{flox/-}; Cdx2-cre mice (FIG. 5D), implying that FST produced in the anterior region of the body is sufficient to maintain FST function in the anterior region even with the lower circulating levels of FST protein. Similarly, unlike the gastrocnemius, the triceps muscles showed no differences in fiber type distribution between Cdx2-cre positive and cre negative mice (FIG. 6A). Moreover, Nile red staining of triceps muscles showed no differences in fluorescence intensity between Cdx2-cre positive and cre negative mice, suggesting that the accumulation of fat droplets seemed to be restricted to the posterior region of the mice in which Cdx2-cre is expressed (FIG. 7B). Finally, whereas RNA-seq analysis of the gastrocnemius muscles identified a total of 633 differentially expressed transcripts between Cdx2-cre positive and cre negative mice, RNA-seq analysis of triceps muscles revealed no differences between Cdx2-cre positive compared to cre negative mice with an adjusted p value less than 0.05. In contrast, fiber type shifts toward glycolytic type 2b fibers and reductions in fat accumulation were seen in both the triceps and gastrocnemius muscles of F66 mice (FIGS. 6A, 7B), reflecting the fact that the Fst overexpressing transgene was expressed in skeletal muscles throughout the body. The simplest interpretation of all these data is that FST acts predominantly, if not exclusively, in a local manner to regulate muscle size, fiber type, and lipid accumulation.

Analysis of the anterior muscles did reveal, however, some systemic effects of targeting Fst in the posterior region. In particular, instead of muscle weights being reduced in the anterior region of Fst^{flox/-}; Cdx2-cre mice, weights of the pectoralis and triceps muscles actually exhibited small, but significant increases compared to cre negative mice (FIG. 5D), which is the opposite of what would be expected upon loss of FST. Although this effect on anterior muscles, which was seen in both males and females, could reflect a variety of physiological changes occurring in the animal as a result of loss of FST production in the posterior half of the body, one possible explanation is that circulating levels of MSTN in Cdx2-cre positive mice are reduced by approximately 35-40% compared to cre negative mice (FIG. 5F). Given that Mstn RNA levels were unchanged in both anterior and posterior muscles of Cdx2-cre positive mice compared to cre negative mice, the drop in circulating MSTN levels likely reflects the reduced amount of total muscle mass in Fst^{flox/-}; Cdx2-cre mice. We have shown that skeletal muscle is by far the predominant source of circulating MSTN protein (22), and in a previous study using a similar approach to target a Mstn^{flox} allele in the posterior region of the body with Cdx2-cre, MSTN was shown to regulate muscle mass both by autocrine/paracrine and by endocrine modes of signaling (40). Hence, the reduction in circulating MSTN in Fst^{flox/-}; Cdx2-cre mice would be predicted to compensate partially for reduced FST with respect to control of muscle mass, and in anteriorly-located muscles, lower circulating MSTN levels would be expected to cause increases in muscle growth.

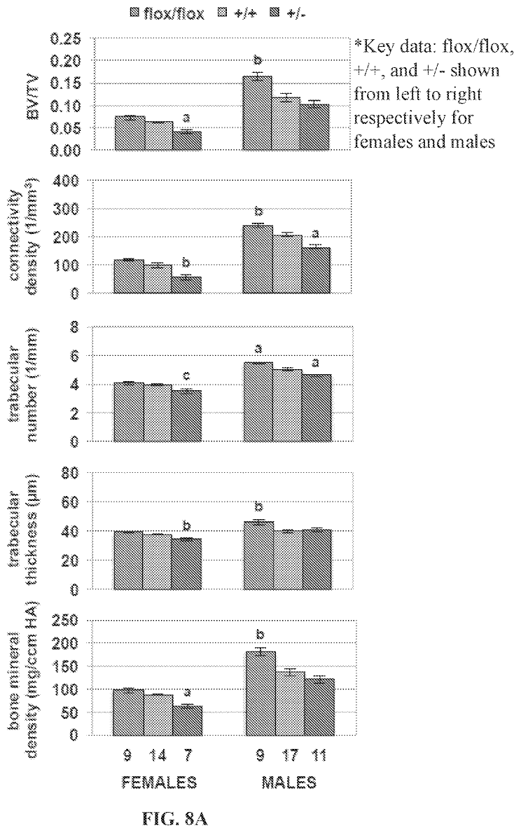

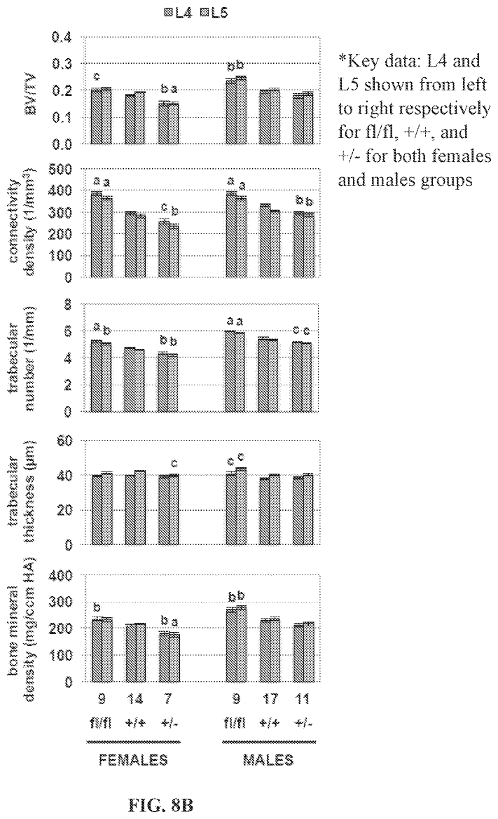

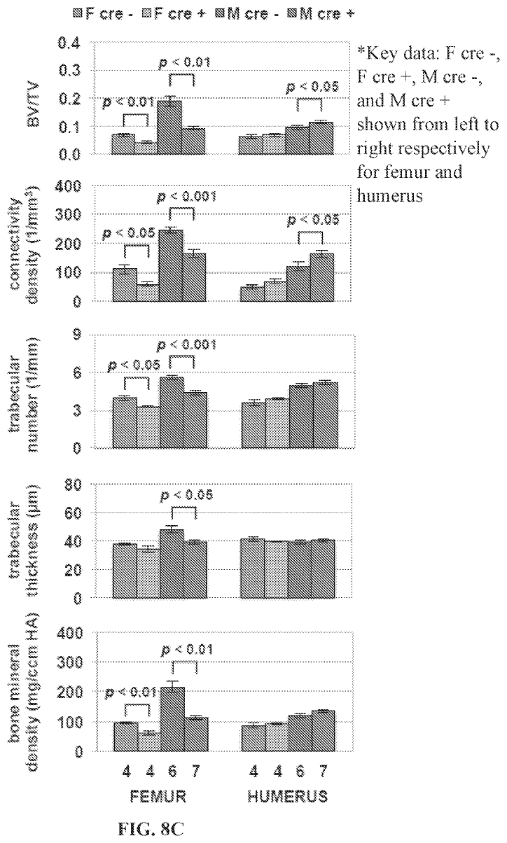

In addition to the key role that the MSTN/activin A signaling pathway plays in regulating muscle mass, several studies have shown that this regulatory system is also important in the regulation of bone structure and bone density. Specifically, systemic administration of a decoy receptor consisting of the extracellular domain of ACVR2B fused to an immunoglobulin Fc domain has been shown to cause significant increases in bone mineral density and various micro-CT parameters, such as BV/TV, connectivity density, and trabecular number (41-45), which are also seen to a milder degree upon targeting Acvr2 and Acvr2b specifically in osteoblasts in vivo (44). Given that FST is capable of blocking the activities of ligands signaling through activin type 2 receptors, the effects of manipulating Fst expression levels on bone density and structure were investigated. As shown in FIGS. 8A-8B, micro-CT analysis of femurs, humeri, and L4 and L5 vertebrae showed opposite trends in Fst^{flox/flox} and Fst^{+/-} mice in comparison to wild-type mice. Specifically, parameters such as bone volume, bone surface, BV/TV, connectivity density, trabecular number, trabecular thickness, and bone mineral density were generally higher in bones of Fst^{flox/flox} mice and were generally lower in bones of Fst^{+/-} mice compared to those of wild-type mice. Many, though not all, of the differences observed among the different genotypes were statistically significant, and the general trends were clearly evident in considering data from both males and females as well as from multiple bones. Hence, as in the case of muscle mass, varying levels of Fst expression also affected bone structure, with reduced levels of Fst expression leading to reduced bone density, demonstrating that FST plays an important, dose-dependent role in regulating bone homeostasis.

To determine whether FST acts locally versus systemically to regulate bone density, the bones of Fst^{flox/-}; Cdx2-cre mice were observed. First, DXA analysis was used to compare different regions of the body in Cdx2-cre positive versus cre negative mice. Bone density by DXA was significantly lower in Cdx2-cre positive compared to cre negative mice in examining bones located in the posterior region of the body, including left and right femurs as well as L2/L3 and L4/L5 vertebrae. In contrast, bone density in either the left or right humerus was similar between Cdx2-cre positive and cre negative mice. Hence, bone density appeared to be affected in bones located in the posterior but not anterior region of the body. To examine bone structure in greater detail, micro-CT analysis was conducted on various bones isolated from these mice. Analysis of femurs showed that bone volume, bone surface, BV/TV, connectivity density, trabecular number, trabecular thickness, and bone mineral density were all significantly reduced in Cdx2-cre positive compared to cre negative mice (FIG. 8C). These decreases were not seen, however, in micro-CT analysis of humeri isolated from these mice, and some of these parameters, such as bone volume, bone surface, BV/TV, and connectivity density, were even slightly increased in Cdx2-cre positive compared to cre negative male mice. Although the basis for these small increases is unclear, the fact that decreases in bone density were seen in femurs but not in humeri of Cdx2-cre positive mice suggests that FST acts locally to regulate bone structure.

The effects on bone seen upon manipulating expression levels of Fst are consistent with prior studies showing that pharmacological inhibition of signaling by systemic administration of the ACVR2B/Fc decoy receptor can cause dramatic changes in bone structure, including increases in bone density, BV/TV, connectivity density, and trabecular number and thickness (41-45). Moreover, targeting Acvr2 either alone or in combination with Acvr2b in osteoblasts in vivo also leads to increases in femoral BV/TV and trabecular number (44). The effects of targeting Acvr2 and Acvr2b in osteoblasts, however, were milder than those seen following treatment with ACVR2B/Fc, and treatment with ACVR2B/Fc was shown to cause even further increases in these parameters in mice in which both receptors were targeted in osteoblasts. These findings imply either that targeting the two activin type 2 receptors, ACVR2 and ACVR2B, may not be sufficient to completely block signaling by key ligands to osteoblasts or that osteoblasts may not be the sole target for these ligands with respect to regulation of bone homeostasis. To address the former possibility, the effect of targeting ALK4 and ALK5, which are the two type 1 receptors utilized by MSTN and activin A in regulating skeletal muscle mass (22), was examined. Specifically, mice carrying floxed alleles for Alk4 and Alk5 were used to examine the effect of simultaneously targeting these type 1 receptors in osteoblasts utilizing the Oc-cre transgene. For comparison, mice in which Acvr2 and Acvr2b were simultaneously targeted using the same Oc-cre transgene, were also analyzed.

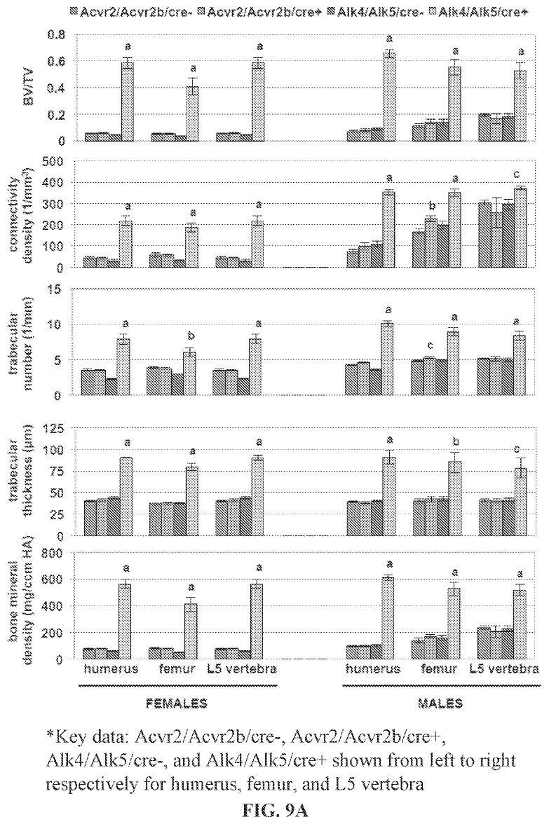

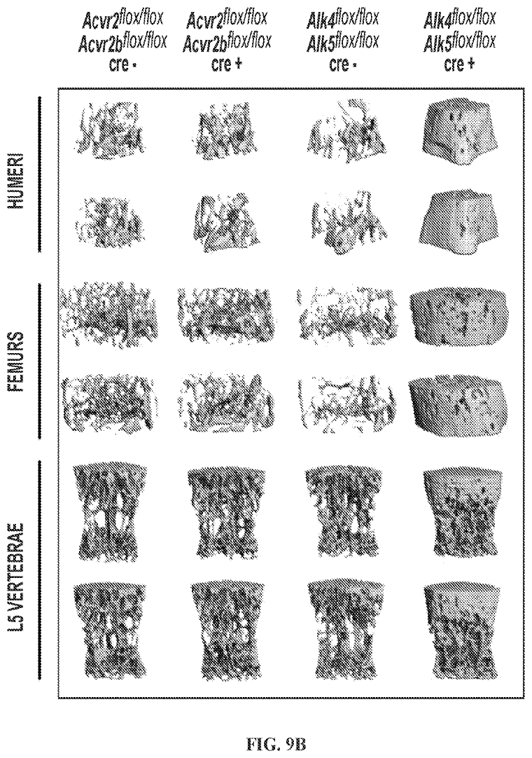

By micro-CT analysis, very few differences were detected in femurs, humeri, or L5 vertebrae of Acvr2^{flox/flox}; Acvr2^{flox/flox}; Oc-cre mice compared to cre negative mice in either males or females (FIG. 9A). In contrast, the effects on bone structure in Alk4^{flox/flox}; Alk5^{flox/flox}; Oc-cre mice were readily apparent upon visual inspection of the micro-CT images (FIG. 9B), and nearly all of the micro-CT parameters showed dramatic differences compared to cre negative control mice in all of the bone groups analyzed in both males and females (FIG. 9A). In females, for example, BV/TV was increased by 12-13-fold and bone mineral density was increased by 8-9-fold in humeri, femurs, and L5 vertebrae of Alk4^{flox/flox}; Alk5^{flox/flox}; Oc-cre mice compared to cre negative mice. Hence, targeting signaling specifically in osteoblasts leads to massive increases in bone volume and density, with the effects of targeting the two type 1 receptors being much more pronounced compared to targeting the two type 2 receptors.

Finally, genetic studies were conducted to determine whether MSTN and activin A are the key ligands signaling through ALK4 and ALK5 to regulate bone. Previous studies have shown that bones of Mstn −/− mice have altered morphology and increased density (for review, see ref. 46), suggesting that MSTN plays either a direct or indirect role in regulating bone structure. The effects of Mstn loss on parameters such as bone density, however, have been reported to be relatively mild compared to the observed effects of targeting the type 1 receptors in osteoblasts. Therefore, the effect of loss of Inhba (encoding the βA subunit of activin A and activin AB) on bone structure was examined. Because complete loss of Inhba leads to embryonic lethality (47), the effect of targeting Inhba in a regionally-restricted manner using the Cdx2-cre transgene was examined, by targeting an Inhba^{flox} allele only in the posterior region of the animal. In order to maximize the potential effects on bone, Mstn was simultaneously targeted using a Mstn^{flox} allele in conjunction with the Cdx2-cre transgene.

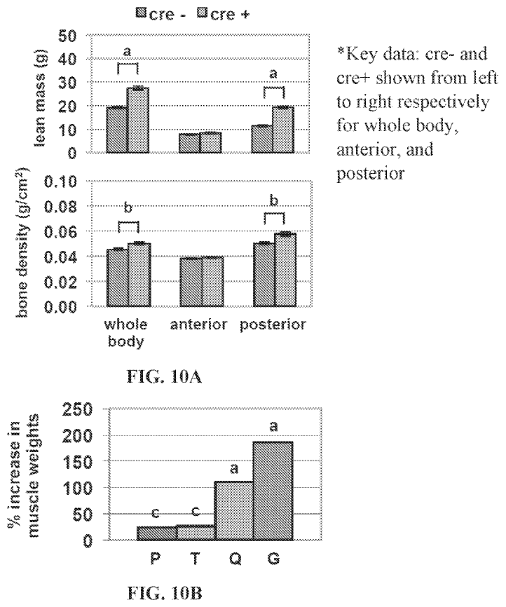

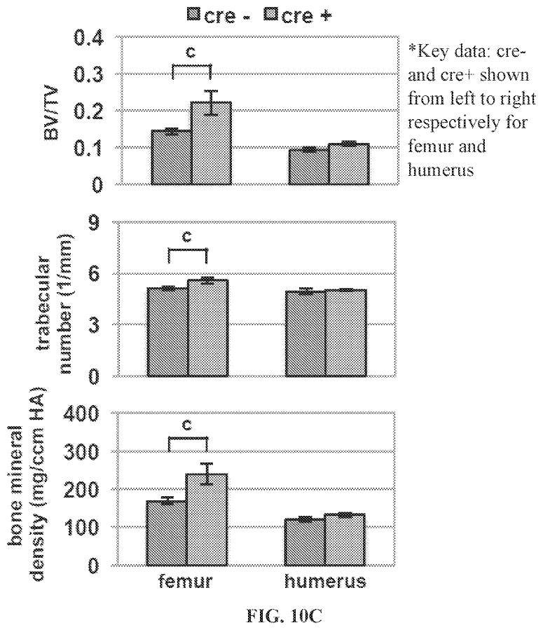

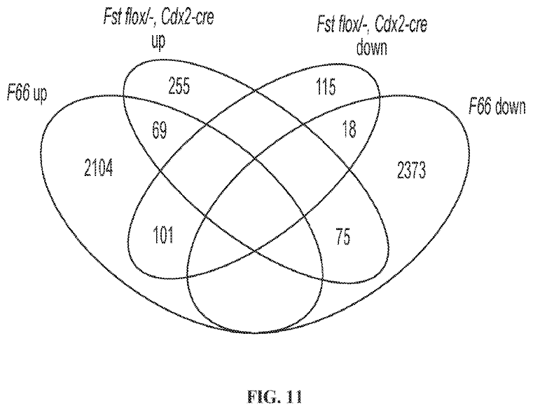

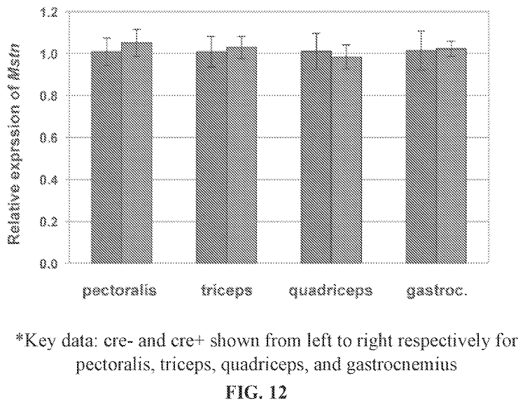

By DXA analysis, lean body mass was significantly increased in Mstn^{flox/flox}; Inhba^{flox/flox}; Cdx2-cre mice compared to that of cre negative mice, which could be attributed almost entirely to increases in the posterior half of the animals (FIG. 10A). Analysis of individual muscles revealed that weights of the quadriceps and gastrocnemius muscles were increased by 111% and 185%, respectively, compared to cre negative mice (FIG. 10B). A previous study showed that targeting Mstn alone in the posterior half of the animal using this same approach led to increases in weights of the quadriceps and gastrocnemius muscles by 73% and 85%, respectively (40). Hence, targeting Mstn and Inhba together led to much more substantial increases in muscle mass compared to targeting Mstn alone, consistent with prior studies documenting that activin A is capable of cooperating with MSTN to limit muscle growth (20, 24-27). Targeting Mstn and Inhba also led to significant changes in bone structure. As in the case of muscle, by DXA analysis, bone mineral density was increased in the posterior half of Mstn^{flox/flox}; Inhba^{flox/flox}; Cdx2-cre mice (FIG. 10A). By micro-CT analysis, parameters such as BV/TV, trabecular number, and bone mineral density were all significantly increased in femurs of Mstn^{flox/flox}; Inhba^{flox/flox}; Cdx2-cre mice (FIG. 10C); the effects, however, were significantly milder than those seen upon targeting Alk4 and Alk5.

To determine whether these ligands act in an autocrine/paracrine or endocrine manner to regulate muscle and bone, tissues isolated from the anterior region of Mstn^{flox/flox}; Inhba^{flox/flox}; Cdx2-cre mice were analyzed. It was previously shown that targeting Mstn in the posterior region leads not only to significant increases in weights of posteriorly-located muscles, but also to modest increases in weights of anteriorly-located muscles (40). As shown in FIG. 10B, weights of pectoralis and triceps muscles, which are located in the anterior region, were increased by approximately 25% in Mstn^{flox/flox}; Inhba^{flox/flox}; Cdx2-cre mice compared to cre negative mice, consistent with a systemic role of circulating MSTN and/or activin A in regulation of muscle mass. In contrast, although trends toward increased BV/TV, trabecular number, and bone mineral density were observed in humeri of male mice (FIG. 10C), none of these differences were significant, and these parameters trended in the opposite direction in female mice. Hence, these ligands appear to act predominantly locally to regulate bone structure.

Presented here are the results of genetic studies demonstrating a critical role for follistatin in regulating both skeletal muscle and bone homeostasis. Using an allelic series corresponding to varying Fst RNA expression levels and circulating FST protein levels, FST was shown to act in an exquisitely dose-dependent manner to regulate both muscle mass and bone density. With respect to muscle, reductions in FST levels lead not only to decreased muscle mass but also a shift toward oxidative fibers and increased intramuscular lipid accumulation, and conversely, increased FST levels lead to increased muscle mass, a shift toward glycolytic fibers, and reduced lipid accumulation. With respect to bone, altered levels of FST lead to significant changes in bone structure, with reductions in FST levels leading to decreased bone density, decreased BV/TV, decreased trabecular number, and decreased trabecular thickness. Moreover, by employing a genetic strategy to target Fst expression only in the posterior region of the animal, it was shown that the effects of Fst loss are mostly restricted to the posterior region, implying that FST acts predominantly in a local manner in both muscle and bone. Our findings suggest that locally produced FST, most likely the FST288 isoform, plays a much more important role than circulating FST, at least with respect to regulation of muscle and bone. We believe that these findings have important implications both with respect to understanding the physiological mechanisms by which muscle and bone homeostasis are regulated by members of the TGF-β family and with respect to the interpretation of studies attempting to correlate circulating levels of FST with various disease states in humans.

Given that the only known mode of action of FST is to bind to and inhibit the activities of members of the TGF-β family of secreted signaling molecules, the effects of altered levels of FST seen in our studies almost certainly reflect altered levels of signaling by various TGF-β family members that are normally targets for FST. Previous studies have demonstrated that blocking the activities of certain TGF-β ligands using a decoy form of the ACVR2B/Fc receptor can lead to dramatic increases in muscle mass (20) and bone density (41-45), implying that the key ligands involved in regulating muscle and bone are likely to be ones capable of binding both FST and ACVR2B/Fc. Two such ligands are MSTN and activin A, and extensive genetic and pharmacological studies have documented the critical role that MSTN and activin A play in regulating muscle growth (1, 20, 24-27). Here, definitive genetic studies are presented showing that targeting these two ligands simultaneously can lead to massive muscle growth. Furthermore, by targeting these ligands only in the posterior region of mice, systemic effects were observed in terms of muscle mass increases in anteriorly-located muscles. These findings, taken together with results of similar studies in which Mstn alone was targeted in the posterior region (40), demonstrate that MSTN and activin A each act in both an autocrine/paracrine and an endocrine manner to regulate muscle mass. In this regard, targeting Fst only in the posterior region led to reduced mass of posteriorly located muscles but actually had the reverse effect of slightly increasing mass of anteriorly located muscles, which is the opposite of what would be predicted for increased signaling by MSTN and/or activin A. This effect in anterior muscles is most likely the result of the dramatically reduced muscle mass in the posterior region of Fst^{flox/-}; Cdx2-cre mice leading to decreased circulating levels of MSTN, which through its endocrine mode of action then affects muscles throughout the body. In contrast to muscle, less is known regarding the key ligands being targeted by FST and ACVR2B/Fc in bone, as definitive genetic studies examining the effects of targeting this subgroup of ligands in bone have not yet been reported. Here, targeting MSTN and activin A led not only to increases in muscle mass, but also to increases in bone density. Although the effects of targeting these two ligands were significant, the magnitude of the effects was considerably lower than what was achieved by targeting the receptors for these ligands, implying that other ligands utilizing these receptors must also play important roles in regulating bone homeostasis. Moreover, unlike in the case of muscle, no systemic effects on bone were detected from targeting these ligands in a regionally-restricted manner, suggesting that these ligands act predominantly locally to regulate bone density.

Whatever the key ligands may be in bone, targeting signaling in osteoblasts is sufficient to cause changes in bone structure, including increases in bone mineral density and density. Although minimal effects of targeting the two type 2 receptors, ACVR2 and ACVR2B, were observed in osteoblasts, the effects on bone structure and density were extensive upon targeting the two type 1 receptors, ALK4 and ALK5. The increases in parameters such as BV/TV and bone mineral density seen upon targeting ALK4 and ALK5 were quite remarkable, reaching levels of 12-13 fold in the case of BV/TV and 8-9 fold in the case of bone mineral density. We previously reported a similar discrepancy in the magnitude of muscle mass increases resulting from targeting these receptors in myofibers (22). In particular, significant increases in muscle mass upon targeting ACVR2 and ACVR2B were observed, but substantially greater increases in muscle mass (up to three-fold) occurred upon targeting ALK4 and ALK5. The differential effects observed upon targeting type 1 versus type 2 receptors in both bone and muscle suggest that the simple model that combinations of ACVR2 and ACVR2B with ALK4 and ALK5 mediate all of the signaling by key TGF-β related ligands in these tissues is likely incomplete. One possible explanation could be that this model is missing a key type 2 receptor or that signaling can occur through some other mechanism. An alternative explanation could be that the spectrum of ligands being blocked by targeting ACVR2 and ACVR2B may be different than those being blocked by targeting ALK4 and ALK5. In this regard, it is known that certain BMPs are capable of utilizing activin type 2 receptors for signaling, and BMP signaling has been shown to have the opposite effect of MSTN/activin A signaling in both muscle (48, 49) and bone (for review, see ref. 50). In addition, TGF-f3 is known to utilize ALK5 for signaling, but TGF-β utilizes a different type 2 receptor, namely TGFBR2, to couple to ALK5.

Further study of this regulatory system will be critical not only for understanding the mechanisms underlying the control of tissue growth but also for developing the most optimal strategies to target this signaling pathway for clinical applications, such as to treat bone loss and muscle loss. In this regard, it will be important to elucidate the precise identities of the key ligands involved in regulating bone homeostasis as well as the precise receptors and other components utilized by these ligands for signaling in both muscle and bone. Moreover, FST is only one of multiple extracellular binding proteins that are capable of binding and inhibiting these ligands, and although considerable work has been done in examining the roles of these binding proteins in muscle, much less has been reported with respect to bone. Understanding the extent to which specific regulatory and signaling components are shared between bone and muscle will be especially important given the extensive ongoing effort among pharmaceutical and biotechnology companies to target this signaling pathway to treat patients with muscle loss and metabolic diseases (for review, see ref. 51), as some of these therapeutic approaches and agents could also be useful to treat bone loss, including conditions characterized by comorbid bone and muscle loss.

The magnitude of the effect observed upon targeting the type 1 receptors in osteoblasts reveals the enormous inherent capacity for bone growth that is normally kept in check by this signaling pathway, and in many respects, these findings are reminiscent of the massive increases in muscle mass observed upon targeting these same receptors in myofibers (22). These findings demonstrate that ligands capable of signaling through these receptors normally function to limit growth of muscle and bone and raise the question as to the extent to which signaling through this pathway may also limit growth of other tissues. Bullough originally proposed the term chalone to describe hypothetical circulating negative growth regulators that function to control tissue size (52). MSTN seems to fulfill all the essential criteria for a chalone for skeletal muscle (53, 54), and our findings raise the possibility that a similar regulatory mechanism with shared signaling components may be operating in other tissues as well.

All animal experiments were carried out in accordance with protocols that were approved by the Institutional Animal Care and Use Committees at the Jackson Laboratory, University of Connecticut School of Medicine, and Johns Hopkins University School of Medicine. Mice carrying floxed alleles for Fst (33), Acvr2 (44), Acvr2b (35), Alk4 (22), Alk5 (55), and Inhba (56) have been described previously. For measurement of muscle weights, individual muscles were dissected from both sides of 10-week old mice, and the average weight was used for each muscle. Circulating protein levels were determined on acid activated serum samples by ELISA using the LSBio F13187 kit (FST) and R&D Systems DGDF80 kit (MSTN). Serial sections (15 μm) were cut transversely through the widest point of the gastrocnemius and triceps muscles using a cryostat. Fiber diameters were measured (as the shortest distance across each fiber passing through the midpoint) from hematoxylin and eosin-stained sections. Measurements were carried out on 250 fibers per muscle, and all data for a given genotype were pooled. Fiber type analysis was carried out using antibodies (BA-D5, SC-71, and BF-F3 for myosin heavy chains type I, IIa, and IIb, respectively) developed by Schiaffino et al. (57) and obtained from the Developmental Studies Hybridoma Bank developed under the auspices of the National Institute of Child Health and Human Development and maintained by the University of Iowa. Nile red staining for lipids was carried out using Nile red dissolved in acetone as described (58). Fluorescence images were taken with a Nikon Ti Eclipse Widefield microscope, and fluorescence was quantified using ImageJ. RNA-seq, DXA scan, and micro-CT analyses were carried out as described previously (45). RNA extraction and analysis by real time quantitative PCR were performed as described previously (40). Histological and RNA-seq analyses were carried out on tissues isolated from male mice so that comparisons could be made with F66 mice, which carry an Fst overexpressing transgene located on the Y chromosome (24).

### REFERENCES

1. A. C. McPherron, A. M. Lawler, & S.-J. Lee, Regulation of skeletal muscle mass in mice by a new TGF-β superfamily member. Nature 387, 83-90 (1997).
2. A. C. McPherron & S.-J. Lee, Double muscling in cattle due to mutations in the myostatin gene. Proc. Natl. Acad. Sci. U.S.A. 94, 12457-12461 (1997).
3. L. Grobet, et al., A deletion in the bovine myostatin gene causes the double-muscled phenotype in cattle. Nature Genet. 17, 71-74 (1997).
4. R. Kambadur, M. Sharma, T. P. L. Smith, & J. J. Bass, Mutations in myostatin (GDF8) in double-muscled Belgian Blue and Piedmontese cattle. Genome Res. 7, 910-915 (1997).
5. A. Clop, et al., A mutation creating a potential illegitimate microRNA target site in the myostatin gene affects muscularity in sheep. Nature Genet. 38, 813-818 (2006).
6. D. S. Mosher, et al., A mutation in the myostatin gene increases muscle mass and enhances racing performance in heterozygote dogs. PLoS Genetics 3, 779-786 (2007).
7. Q. Lv, et al., Efficient Generation of Myostatin Gene Mutated Rabbit by CRISPR/Cas9. Sci. Rep. 6, 25029 (2016).
8. H. Gu, et al., Establishment and phenotypic analysis of an Mstn knockout rat. Biochem. Biophys. Res. Commun. 477, 115-122 (2016).
9. K. Wang, et al., CRISPR/Cas9-mediated knockout of myostatin in Chinese indigenous Erhualian pigs. Transgenic Res. 26, 799-805 (2017).
10. Z. He, et al., Use of CRISPR/Cas9 technology efficiently targeted goat myostatin through zygotes microinjection resulting in double-muscled phenotype in goats. Biosci. Rep. 38, BSR20180742 (2018).
11. M. Schuelke, et al., Myostatin mutation associated with gross muscle hypertrophy in a child. N. Engl. J. Med. 350, 2682-2688 (2004).
12. S.-J. Lee & A. C. McPherron, Regulation of myostatin activity and muscle growth. Proc. Natl. Acad. Sci. U.S.A. 98, 9306-9311 (2001).
13. J. J. Hill, et al., The myostatin propeptide and the follistatin-related gene are inhibitory binding proteins of myostatin in normal serum. J. Biol. Chem. 277, 40735-40741 (2002).
14. J. J. Hill, Y. Qiu, R. M. Hewick, & N. M. Wolfman, Regulation of myostatin in vivo by growth and differentiation factor-associated serum protein-1: a novel protein with protease inhibitor and follistatin domains. Mol. Endocrinol. 17, 1144-1154 (2003).
15. Y.-S. Lee & S.-J. Lee, Regulation of GDF-11 and myostatin activity by GASP-1 and GASP-2. Proc. Natl. Acad. Sci. U.S.A. 110, E3713-22 (2013).
16. K. Kondas, G. Szlama, M. Trexler, & L. Patthy, Both WFIKKN1 and WFIKKN2 have high affinity for growth and differentiation factors 8 and 11. J. Biol. Chem. 283, 23677-23684 (2008).
17. R. S. Thies, et al., GDF-8 propeptide binds to GDF-8 and antagonizes biological activity by inhibiting GDF-8 receptor binding. Growth Factors 18, 251-259 (2001).
18. N. M. Wolfman, et al., Activation of latent myostatin by the BMP-1/tolloid family of metalloproteinases. Proc. Natl. Acad. Sci. U.S.A. 100, 15842-15846 (2003).
19. S.-J. Lee, Genetic analysis of the role of proteolysis in the activation of latent myostatin. PLoS One 3, e1628 (2008).
20 S.-J. Lee, et al., Regulation of muscle growth by multiple ligands signaling through activin type 2 receptors. Proc. Natl. Acad. Sci. U.S.A. 102, 18117-18122 (2005).
21. F. Morvan, et al., Blockade of activin type 2 receptors with a dual anti-ActRIIA/IIB antibody is critical to promote maximal skeletal muscle hypertrophy. Proc. Natl. Acad. Sci. U.S.A. 114, 12448-12453 (2017).
22. S.-J. Lee, et al., Functional redundancy of type 1 and type 2 receptors in the regulation of skeletal muscle growth by myostatin and activin A. Proc. Natl. Acad. Sci. U.S.A. 117, 30907-30917 (2020).
23. A. Rebbapragada, H. Benchabane, J. Wrana, A. J. Celeste, & L. Attisano, Myostatin signals through a transforming growth factor β-like signaling pathway to block adipogenesis. Mol. Cell. Biol. 23, 7230-7242 (2003).
24. S.-J. Lee, Quadrupling muscle mass in mice by targeting TGF-β signaling pathways. PloS One 2, e789 (2007).
25. S.-J. Lee, et al., Regulation of muscle mass by follistatin and activins. Mol. Endocrinol. 24, 1998-2008 (2010).
26. E. Latres, et al. Activin A more prominently regulates muscle mass in primates than does GDF8. Nat. Commun. 8, 15153 (2017).
27. J. L. Chen, et al., Specific targeting of TGF-β family ligands demonstrates distinct roles in the regulation of muscle mass in health and disease. Proc. Natl. Acad. Sci. U.S.A. 114, E5266-E5275 (2017).
28. N. Ueno, et al., Isolation and partial characterization of follistatin: a single-chain Mr monomeric protein that inhibits the release of follicle-stimulating hormone. Proc. Natl. Acad. Sci. U.S.A. 84, 8282-8286 (1987).
29. T. Nakamura, et al., Activin-binding protein from rat ovary is follistatin. Science 247, 836-838 (1990).
30. N. Ling, et al., Pituitary FSH is released by a heterodimer of the beta-subunits from the two forms of inhibin. Nature 321, 779-782 (1986).
31. K. Sugino, et al., Molecular heterogeneity of follistatin, an activin-binding protein. Higher affinity of the carboxyl-terminal truncated forms for heparan sulfate proteoglycans on the ovarian granulosa cell. J. Biol. Chem. 268, 15579-15587 (1993).
32. M. M. Matzuk, et al., Multiple defects and perinatal death in mice deficient in follistatin. Nature 374, 360-363 (1995).
33. C. J. Jorgez, M. Klysik, S. P. Jamin, R. R. Behringer, & M. M. Matzuk, Granulosa cell-specific inactivation of follistatin causes female fertility defects. Mol. Endocrinol. 18, 953-967 (2004).
34. A. C. McPherron, T. V. Huynh, & S.-J. Lee, Redundancy of myostatin and growth/differentiation factor 11. BMC Dev. Biol. 9, 24-32 (2009).
35. S.-J. Lee, et al., Role of satellite cells versus myofibers in muscle hypertrophy induced by inhibition of the myostatin/activin signaling pathway. Proc. Natl. Acad. Sci. U.S.A. 109, E2353-2360 (2012).
36. T. Hinoi, et al., Mouse model of colonic adenoma-carcinoma progression based on somatic Apc inactivation. Cancer Res. 67, 9721-9730 (2007).
37. Y. Komiya, et al., Mouse soleus (slow) muscle shows greater intramyocellular lipid droplet accumulation than EDL (fast) muscle: fiber type-specific analysis. J. Muscle Res. Cell. Motil. 38, 163-173 (2017).
38. D. Pette, & R. S. Staron, Cellular and molecular diversities of mammalian skeletal muscle fibers. Rev. Physiol. Biochem. Pharmacol. 116, 1-76 (1990).
39. S. Schiaffino, & C. Reggiani, Fiber types in mammalian skeletal muscles. Physiol. Rev. 91, 1447-531 (2011).
40. Y.-S. Lee, T.V. Huynh TV, & S.-J. Lee, Paracrine and endocrine modes of myostatin action. J. Appl. Physiol. 120, 592-598 (2016).
41. A. Koncarevic, et al., A soluble activin receptor type 2b prevents the effects of androgen deprivation on body composition and bone health. Endocrinology 151, 4289-4300 (2010).
42. C. S. Chiu, et al., Increased muscle force production and bone mineral density in ActRIIB-Fc-treated mature rodents. J. Gerontol. A Biol. Sci. Med. Sci. 68, 1181-1192 (2013).
43. P. Bialek, et al., A myostatin and activin decoy receptor enhances bone formation in mice. Bone 60, 162-171 (2014).
44. B.C. Goh, et al. Activin receptor type 2A (ACVR2A) functions directly in osteoblasts as a negative regulator of bone mineral density. J. Biol. Chem. 292, 13809-13822 (2017).
45. S.-J. Lee, et al., Targeting myostatin/activin A protects against skeletal muscle and bone loss during spaceflight. Proc Natl Acad Sci USA 117:23942-23951 (2020).
46. M. N. Elkasrawy M N & M. W. Hamrick, Myostatin (GDF-8) as a key factor linking muscle mass and bone structure. J. Musculoskelet. Neuronal. Interact. 10, 56-63 (2010).
47. M. M. Matzuk, et al., Functional analysis of activins during mammalian development. Nature 374, 354-356 (1995).
48. C. E. Winbanks, et al., The bone morphogenetic protein axis is a positive regulator of skeletal muscle mass. J. Cell. Biol. 203, 345-357 (2013).
49. R. Sartori, et al., BMP signaling controls muscle mass. Nat. Genet. 45, 1309-1318 (2013).
50. J. W. Lowery & V. Rosen, The BMP Pathway and Its Inhibitors in the Skeleton. Physiol Rev. 98, 2431-2452 (2018).
51. S.-J. Lee, Targeting the myostatin signaling pathway to treat muscle loss and metabolic dysfunction. J. Clin. Invest. 131, e148372 (2021).
52. W. S. Bullough, The control of mitotic activity in adult mammalian tissues. Biol. Rev. 37, 307-342 (1962).
53. S.-J. Lee, Regulation of muscle mass by myostatin. Annu. Rev. Cell Dev. Biol. 20, 61-86 (2004).
54. S.-J. Lee, Extracellular Regulation of Myostatin: A Molecular Rheostat for Muscle Mass. Immunol. Endocr. Metab. Agents Med. Chem. 10, 183-194 (2010).
55. J. Larsson, et al., Abnormal angiogenesis but intact hematopoietic potential in TGF-beta type 1 receptor-deficient mice. EMBO J. 20, 1663-1673 (2001).
56. S. A. Pangas, et al., Intraovarian activins are required for female fertility. Mol. Endocrinol. 21, 2458-2471 (2007).
57. S. Schiaffino, et al., Three myosin heavy chain isoforms in type 2 skeletal muscle fibres. J. Muscle Res. Cell Motil. 10, 197-205 (1989).
58. S. D. Fowler & P. Greenspan, Application of Nile red, a fluorescent hydrophobic probe, for the detection of neutral lipid deposits in tissue sections: comparison with oil red O. J. Histochem. Cytochem. 33, 833-836 (1985).

SEQUENCES
Human ALK4
(SEQ ID NO: 1)
GGGCGCTGCTGGGCTGCGGCGGCGGCGGCGGCGGCGGTGG
TTACTATGGCGGAGTCGGCCGGAGCCTCCTCCTTCTTCCC
CCTTGTTGTCCTCCTGCTCGCCGGCAGCGGCGGGTCCGGG
CCCCGGGGGGTCCAGGCTCTGCTGTGTGCGTGCACCAGCT
GCCTCCAGGCCAACTACACGTGTGAGACAGATGGGGCCTG
CATGGTTTCCATTTTCAATCTGGATGGGATGGAGCACCAT
GTGCGCACCTGCATCCCCAAAGTGGAGCTGGTCCCTGCCG
GGAAGCCCTTCTACTGCCTGAGCTCGGAGGACCTGCGCAA
CACCCACTGCTGCTACACTGACTACTGCAACAGGATCGAC
TTGAGGGTGCCCAGTGGTCACCTCAAGGAGCCTGAGCACC
CGTCCATGTGGGGCCCGGTGGAGCTGGTAGGCATCATCGC
CGGCCCGGTGTTCCTCCTGTTCCTCATCATCATCATTGTT
TTCCTTGTCATTAACTATCATCAGCGTGTCTATCACAACC
GCCAGAGACTGGACATGGAAGATCCCTCATGTGAGATGTG
TCTCTCCAAAGACAAGACGCTCCAGGATCTTGTCTACGAT
CTCTCCACCTCAGGGTCTGGCTCAGGGTTACCCCTCTTTG
TCCAGCGCACAGTGGCCCGAACCATCGTTTTACAAGAGAT
TATTGGCAAGGGTCGGTTTGGGGAAGTATGGCGGGGCCGC
TGGAGGGGTGGTGATGTGGCTGTGAAAATATTCTCTTCTC
GTGAAGAACGGTCTTGGTTCAGGGAAGCAGAGATATACCA
GACGGTCATGCTGCGCCATGAAAACATCCTTGGATTTATT
GCTGCTGACAATAAAGCAGACTGCTCATTCCTCACATTGC
CATGGGAAGTTGTAATGGTCTCTGCTGCCCCCAAGCTGAG
GAGCCTTAGACTCCAATACAAGGGAGGAAGGGGAAGAGCA
AGATTTTTATTCCCACTGAATAATGGCACCTGGACACAGC
TGTGGCTTGTTTCTGACTATCATGAGCACGGGTCCCTGTT
TGATTATCTGAACCGGTACACAGTGACAATTGAGGGGATG
ATTAAGCTGGCCTTGTCTGCTGCTAGTGGGCTGGCACACC
TGCACATGGAGATCGTGGGCACCCAAGGGAAGCCTGGAAT
TGCTCATCGAGACTTAAAGTCAAAGAACATTCTGGTGAAG
AAAAATGGCATGTGTGCCATAGCAGACCTGGGCCTGGCTG
TCCGTCATGATGCAGTCACTGACACCATTGACATTGCCCC
GAATCAGAGGGTGGGGACCAAACGATACATGGCCCCTGAA
GTACTTGATGAAACCATTAATATGAAACACTTTGACTCCT
TTAAATGTGCTGATATTTATGCCCTCGGGCTTGTATATTG
GGAGATTGCTCGAAGATGCAATTCTGGAGGAGTCCATGAA
GAATATCAGCTGCCATATTACGACTTAGTGCCCTCTGACC
CTTCCATTGAGGAAATGCGAAAGGTTGTATGTGATCAGAA
GCTGCGTCCCAACATCCCCAACTGGTGGCAGAGTTATGAG
GCACTGCGGGTGATGGGGAAGATGATGCGAGAGTGTTGGT
ATGCCAACGGCGCAGCCCGCCTGACGGCCCTGCGCATCAA
GAAGACCCTCTCCCAGCTCAGCGTGCAGGAAGACGTGAAG
ATCTAACTGCTCCCTCTCTCCACACGGAGCTCCTGGCAGC
GAGAACTACGCACAGCTGCCGCGTTGAGCGTACGATGGAG
GCCTACCTCTCGTTTCTGCCCAGCCCTCTGTGGCCAGGAG
CCCTGGCCCGCAAGAGGGACAGAGCCCGGGAGAGACTCGC
TCACTCCCATGTTGGGITTGAGACAGACACCTTTTCTATT
TACCTCCTAATGGCATGGAGACTCTGAGAGCGAATTGTGT
GGAGAACTCAGTGCCACACCTCGAACTGGTTGTAGTGGGA
AGTCCCGCGAAACCCGGTGCATCTGGCACGTGGCCAGGAG
CCATGACAGGGGCGCTTGGGAGGGGCCGGAGGAACCGAGG
TGTTGCCAGTGCTAAGCTGCCCTGAGGGTTTCCTTCGGGG
ACCAGCCCACAGCACACCAAGGTGGCCCGGAAGAACCAGA
AGTGCAGCCCCTCTCACAGGCAGCTCTGAGCCGCGCTTTC
CCCTCCTCCCTGGGATGGACGCTGCCGGGAGACTGCCAGT
GGAGACGGAATCTGCCGCTTTGTCTGTCCAGCCGTGTGTG
CATGTGCCGAGGTGCGTCCCCCGTTGTGCCTGGTTCGTGC
CATGCCCTTACACGTGCGTGTGAGTGTGTGTGTGTGTCTG
TAGGTGCGCACTTACCTGCTTGAGCTTTCTGTGCATGTGC
AGGTCGGGGGTGTGGTCGTCATGCTGTCCGTGCTTGCTGG
TGCCTCTTTTCAGTAGTGAGCAGCATCTAGTTTCCCTGGT
GCCCTTCCCTGGAGGTCTCTCCCTCCCCCAGAGCCCCTCA
TGCCACAGTGGTACTCTGTGTCTGGCAGGCTACTCTGCCC
ACCCCAGCATCAGCACAGCTCTCCTCCTCCATCTCAGACT
GTGGAACCAAAGCTGGCCCAGTTGTCCATGACAAAAGAGG
CTTTTGGGCCAAAATGTGAGGGTGGTGGGTGGGATGGGCA
GGGAAGGAATCCTGGTGGAAGTCTTGGGTGTTAGTGTCAG
CCATGGGAAATGAGCCAGCCCAAGGGCATCATCCTCAGCA
GCATCGAGGAAGGGCCGAGGAATGTGAAGCCAGATCTCGG
GACTCAGATTGGAATGTTACATCTGTCTTTCATCTCCCAG
ATCCTGGAAACAGCAGTGTATATTTTTGGTGGTGGTGGGT
TTGGGGTGGGGAAGGGAAGGGCGGGCAAGGAGTGGGGAGG
GAGTCTGGGGTGGGAGGGAGGCATCTGCATGGGTCTTCTT
TTACTGGACTGTCTGATCAGGGTGGAGGGAAGGTGAGAGG
TTTGCATCCACTTCAGGAGCCCTACTGAAGGGAACAGCCT
GAGCCGAACATGTTATTTAACCTGAGTATAGTATTTAACG
AAGCCTAGAAGCACGGCTGTGGGTGGTGATTIGGTCAGCA
TATCTTAGGTATATAATAACTTTGAAGCCATAACTTTTAA
CTGGAGTGGTTTGATTTCTTTTTTTAATTTTATTGGGAGG
GTTTGGATTTTAACTTTTTTTAATGTTGTTAAATATTAAG
TTTTTGTAAAAGGAAAACCATCTCTGTGATTACCTCTCAA
TCTATTTGTTTTTAAAGAAATCCCTAAAAAAAAAAATTAT
CCAATTGAACGCACATAGCTCAATCACACTGGAAATGTTT
GTCCTTGCACCTGAGCCTGTTCCCACTCAGCAGTGAGAGT
TCCTCTTTGCCCTGAGGCTCAGTCTCTCTCGTATTTTGTC
CCCACCCCCAATTCCTTGAGTGGTTTTTGCTCTAGGGCCC
TTTCTTGCACTGTCCAGCTGGTTGTACCCTCTCCAGGCAT
TTATTCAACAAATGTGGGTGAAGTGCCTGCTGGGTGCCAG
GTGCTGGGAATACATCTGTGGACAAGACATGCTTGGGTCC
TACTCCTGGAGCACTGTAAAAAGAGCTGATTCAAGTAAGT
AGATGCCTGTTTTGAGACCAGAAGGTTTCATAATTGGTTC
TACGACCCTTTTGAGCCTAGAATTATTGTTCTTATATAAG
ATCACTGAAGAAAGAGGAACCCCCACAACCCCCTCCACAA
AGAGACCAGGGGGGGGTGATGAGACCTGGGGTTTAGAACC
CCAGGTGAGACCTCAAATCACTGCATTCATTCTGAGCCCC
CTTCCTGTCCCCAGGGGAGGTGTATTGTGTATGTAGCCTT
AGAGCATCTCTGCCTCCAACCCAGCAGTTCTCTGCCAAAG
CTTGTGGAGGAGGGAGAGCCCTGTCCCTGCCCTCAGGCTC
CCCAGTGCTCCTGGCCCTTCTATTTATTTGACTGATTATT
GCTTCTTTCCTTGCATTAAAGGAGATCTTCCCCTAACCTT
TGGGCCAATTTACTGGCCACTAATTTCGTTTAAATACCAT
TGTGTCATTGGGGGGACCGTCTTTACCCCTGCTGACCTCC
CACCTATCCGCCCTGCAGCAGAACCTTGGCGGTTTATAGG
TAATGATGGAACTTAGACTCCTCTTCCCAGAGTCACAAGT
AGCCTCTGGGATCTGCCAACACACGTCCACTCCCAAGCCA
CTAGCCCACTCCCCAGTTGGCCCTTCTGCCCTTACCCCAC
ACACAGTCCAACTCTTCCACCTCTGGGGAAGATGGAGCAG
GTCTTTGGGAAGCTCCCACACCCACCTCTGCCACTCTTAA
CACTAAGTGAGAGTTGGGGAGAAACTGAAGCCGTGTTTTT
GGCCCCCCGAGGCTAACCCTGATCCATAGTGCTACCTGCA
CCTCTGGATTCTGGATTCACAGACCAAGTCCAAGCCCGTT
CTTACGTCGCCATAAAGGCCCCCGAACGGCATTCTCGGTA
CTTCTGTTTGTTTTTGTACATTTTATTAGAAAGGACTGTA
AAATAGCCACTTAGACACTTTACCTCTTCAGTATGCAAAT
GTAAATAAATTGTAATATAGGAAATCTTTTGTTTTAATAT
AAGAATGAGCCTGTCCAATTTCTGCTGTACATTATTAAAA
GTTTTATTCACAGA
Human ALK4
(SEQ ID NO: 2)
MAESAGASSFFPLVVLLLAGSGGSGPRGVQALLCACTSCL
QANYTCETDGACMVSIFNLDGMEHHVRTCIPKVELVPAGK
PFYCLSSEDLRNTHCCYTDYCNRIDLRVPSGHLKEPEHPS
MWGPVELVGIIAGPVFLLFLIIIIVFLVINYHQRVYHNRQ
RLDMEDPSCEMCLSKDKTLQDLVYDLSTSGSGSGLPLFVQ
RTVARTIVLQEIIGKGRFGEVWRGRWRGGDVAVKIFSSRE
ERSWFREAEIYQTVMLRHENILGFIAADNKDNGTWTQLWL
VSDYHEHGSLFDYLNRYTVTIEGMIKLALSAASGLAHLHM
EIVGTQGKPGIAHRDLKSKNILVKKNGMCAIADLGLAVRH
DAVIDTIDIAPNQRVGTKRYMAPEVLDETINMKHFDSFKC
ADIYALGLVYWEIARRCNSGGVHEEYQLPYYDLVPSDPSI
EEMRKVVCDQKLRPNIPNWWQSYEALRVMGKMMRECWYAN
GAARLTALRIKKTLSQLSVQEDVKI
Human ALK5
(SEQ ID NO: 3)
GGCGGCTAGGGAGGTGGGGCGAGGCGAGGTTTGCTGGGGT
GAGGCAGCGGCGCGGCCGGGCCGGGCCGGGCCACAGGCGG
TGGCGGCGGGACCATGGAGGCGGCGGTCGCTGCTCCGCGT
CCCCGGCTGCTCCTCCTCGTGCTGGCGGCGGCGGCGGCGG
CGGCGGCGGCGCTGCTCCCGGGGGCGACGGCGTTACAGTG
TTTCTGCCACCTCTGTACAAAAGACAATTTTACTTGTGTG
ACAGATGGGCTCTGCTTTGTCTCTGTCACAGAGACCACAG
ACAAAGTTATACACAACAGCATGTGTATAGCTGAAATTGA
CTTAATTCCTCGAGATAGGCCGTTTGTATGTGCACCCTCT
TCAAAAACTGGGTCTGTGACTACAACATATTGCTGCAATC
AGGACCATTGCAATAAAATAGAACTTCCAACTACTGGTTT
ACCATTGCTTGTTCAGAGAACAATTGCGAGAACTATTGTG
TTACAAGAAAGCATTGGCAAAGGTCGATTTGGAGAAGTTT
GGAGAGGAAAGTGGCGGGGAGAAGAAGTTGCTGTTAAGAT
ATTCTCCTCTAGAGAAGAACGTTCGTGGTTCCGTGAGGCA
GAGATTTATCAAACTGTAATGTTACGTCATGAAAACATCC
TGGGATTTATAGCAGCAGACAATAAAGACAATGGTACTTG
GACTCAGCTCTGGTTGGTGTCAGATTATCATGAGCATGGA
TCCCTTTTTGATTACTTAAACAGATACACAGTTACTGTGG
AAGGAATGATAAAACTTGCTCTGTCCACGGCGAGCGGTCT
TGCCCATCTTCACATGGAGATTGTTGGTACCCAAGGAAAG
CCAGCCATTGCTCATAGAGATTTGAAATCAAAGAATATCT
TGGTAAAGAAGAATGGAACTTGCTGTATTGCAGACTTAGG
ACTGGCAGTAAGACATGATTCAGCCACAGATACCATTGAT
ATTGCTCCAAACCACAGAGTGGGAACAAAAAGGTACATGG
CCCCTGAAGTTCTCGATGATTCCATAAATATGAAACATTT
TGAATCCTTCAAACGTGCTGACATCTATGCAATGGGCTTA
GTATTCTGGGAAATTGCTCGACGATGTTCCATTGGTGGAA
TTCATGAAGATTACCAACTGCCTTATTATGATCTTGTACC
TTCTGACCCATCAGTTGAAGAAATGAGAAAAGTTGTTTGT
GAACAGAAGTTAAGGCCAAATATCCCAAACAGATGGCAGA
GCTGTGAAGCCTTGAGAGTAATGGCTAAAATTATGAGAGA
ATGTTGGTATGCCAATGGAGCAGCTAGGCTTACAGCATTG
CGGATTAAGAAAACATTATCGCAACTCAGTCAACAGGAAG
GCATCAAAATGTAATTCTACAGCTTTGCCTGAACTCTCCT
TTTTTCTTCAGATCTGCTCCTGGGTTTTAATTTGGGAGGT
CAATTGTTCTACCTCACTGAGAGGGAACAGAAGGATATTG
CTTCCTTTTGCAGCAGTGTAATAAAGTCAATTAAAAACTT
CCCAGGATTTCTTTGGACCCAGGAAACAGCCATGTGGGTC
CTTTCTGTGCACTATGAACGCTTCTTTCCCAGGACAGAAA
ATGTGTAGTCTACCTTTATTTTTTATTAACAAAACTTGTT
TTTTAAAAAGATGATTGCTGGTCTTAACTTTAGGTAACTC
TGCTGTGCTGGAGATCATCTTTAAGGGCAAAGGAGTTGGA
TTGCTGAATTACAATGAAACATGTCTTATTACTAAAGAAA
GTGATTTACTCCTGGTTAGTACATTCTCAGAGGATTCTGA
ACCACTAGAGTTTCCTTGATTCAGACTTTGAATGTACTGT
TCTATAGTTTTTCAGGATCTTAAAACTAACACTTATAAAA
CTCTTATCTTGAGTCTAAAAATGACCTCATATAGTAGTGA
GGAACATAATTCATGCAATTGTATTTTGTATACTATTATT
GTTCTTTCACTTATTCAGAACATTACATGCCTTCAAAATG
GGATTGTACTATACCAGTAAGTGCCACTTCTGTGTCTTTC
TAATGGAAATGAGTAGAATTGCTGAAAGTCTCTATGTTAA
AACCTATAGTGTTTGAATTCAAAAAGCTTATTTATCTGGG
TAACCCAAACTTTTTCTGTTTTGTTTTTGGAAGGGTTTTT
GTGGTATGTCATTTGGTATTCTATTCTGAAAATGCCTTTC
TCCTACCAAAATGTGCTTAAGCCACTAAAGAAATGAAGTG
GCATTAATTAGTAAATTATTAGCATGGTCATGTTTGAATA
TTCTCACATCAAGCTTTTGCATTTTAATTGTGTTGTCTAA
GTATACTTTTAAAAAATCAAGTGGCACTCTAGATGCTTAT
AGTACTTTAATATTTGTAGCATACAGACTAATTTTTCTAA
AAGGGAAAGTCTGTCTAGCTGCTTGTGAAAAGTTATGTGG
TATTCTGTAAGCCATTTTTTTCTTTATCTGTTCAAAGACT
TATTTTTTAAGACATGAATTACATTTAAAATTAGAATATG
GTTAATATTAAATAATAGGCCTTTTTCTAGGAAGGCGAAG
GTAGTTAATAATTTGAATAGATAACAGATGTGCAAGAAAG
TCACATTTGTTATGTATGTAGGAGTAAACGTTCGGTGGAT
CCTCTGTCTTTGTAACTGAGGTTAGAGCTAGTGTGGTTTT
GAGGTCTCACTACACTTTGAGGAAGGCAGCTTTTAATTCA
GTGTTTCCTTATGTGTGCGTACATTGCAACTGCTTACATG
TAATTTATGTAATGCATTCAGTGCACCCTTGTTACTTGGG
AGAGGTGGTAGCTAAAGAACATTCTGAGTATAGGTTTTTC
TCCATTTACAGATGTCTTTGGTCAAATATTGAAAGCAAAC
TTGTCATGGTCTTCTTACATTAAGTTGAAACTAGCTTATA
ATAACTGGTTTTTACTTCCAATGCTATGAAGTCTCTGCAG
GGCTTTTACAGTTTTCGAAGTCCTTTTATCACTGTGATCT
TATTCTGAGGGGAGAAAAAACTATCATAGCTCTGAGGCAA
GACTTCGACTTTATAGTGCTATCAGTTCCCCGATACAGGG
TCAGAGTAACCCATACAGTATTTTGGTCAGGAAGAGAAAG
TGGCCATTTACACTGAATGAGTTGCATTCTGATAATGTCT
TATCTCTTATACGTAGAATAAATTTGAAAGACTATTTGAT
CTTAAAACCAAAGTAATTTTAGAATGAGTGACATATTACA
TAGGAATTTAGTGTCAATTTCATGTGTTTAAAAACATCAT
GGGAAAAATGCTTAGAGGTTACTATTTTGACTACAAAGTT
GAGTTTTTTTCTGTAGTTACCATAATTTCATTGAAGCAAA
TGAATGAGTTTGAGAGGTTTGTTTTTATAGTTGTGTTGTA
TTACTTGTTTAATAATAATCTCTAATTCTGTGATCAGGTA
CTTTTTTTGTGGGGGTTTTTTTTTTGTTTTTTTTTTTTTG
TTGTTGTTTTTGGGCCATTTCTAAGCCTACCAGATCTGCT
TTATGAAATCCAGGGGACCAATGCATTTTATCACTAAAAC
TATTTTTATATAATTTTAAGAATATACCAAAAGTTGTCTG
ATTTAAAGTTGTAATACATGATTTCTCACTTTCATGTAAG
GTTATCCACTTTTGCTGAAGATATTTTTTATTGAATCAAA
GATTGAGTTACAATTATACTTTTCTTACCTAAGTGGATAA
AATGTACTTTTGATGAATCAGGGAATTTTTTTAAAGTTGG
AGTTTAGTTCTAAATTGACTTTACGTATTACTGCAGTTAA
TTCCTTTTTTGGCTAGGGATGGTTTGATAAACCACAATTG
GCTGATATTGAAAATGAAAGAAACTTAAAAGGTGGGATGG
ATCATGATTACTGTCGATAACTGCAGATAAATTTGATTAG
AGTAATAATTTTGTCATTTAAAAACACAGTTGTTTATACT
GCCCATCCTAGGATGCTCACCTTCCAAGATTCAACGTGGC
TAAAACATCTTCTGGTAAATTGTGCGTCCATATTCATTTT
GTCAGTAGCCAGGAGAAATGGGGATGGGGGAAATACGACT
TAGTGAGGCATAGACATCCCTGGTCCATCCTTTCTGTCTC
CAGCTGTTTCTTGGAACCTGCTCTCCTGCTTGCTGGTCCC
TGACGCAGAGACCGTTGCCTCCCCCACAGCCGTTTGACTG
AAGGCTGCTCTGGAGACCTAGAGTAAAACGGCTGATGGAA
GTTGTGGGACCCACTTCCATTTCCTTCAGTCATTAGAGGT
GGAAGGGAGGGGTCTCCAAGTTTGGAGATTGAGCAGATGA
GGCTTGGGATGCCCCTGCTTTGACTTCAGCCATGGATGAG
GAGTGGGATGGCAGCAAGGTGGCTCCTGTGGCAGTGGAGT
TGTGCCAGAAACAGTGGCCAGTTGTATCGCCTATAAGACA
GGGTAAGGTCTGAAGAGCTGAGCCTGTAATTCTGCTGTAA
TAATGATAGTGCTCAAGAAGTGCCTTGAGTTGGTGTACAG
TGCCATGGCCATCAAGAATCCCAGATTTCAGGTTTTATTA
CAAAATGTAAGTGGTCACTTGGCGATTTTGTAGTACATGC
ATGAGTTACCTTTTTTCTCTATGTCTGAGAACTGTCAGAT
TAAAACAAGATGGCAAAGAGATCGTTAGAGTGCACAACAA
AATCACTATCCCATTAGACACATCATCAAAAGCTTATTTT
TATTCTTGCACTGGAAGAATCGTAAGTCAACTGTTTCTTG
ACCATGGCAGTGTTCTGGCTCCAAATGGTAGTGATTCCAA
ATAATGGTTCTGTTAACACTTTGGCAGAAAATGCCAGCTC
AGATATTTTGAGATACTAAGGATTATCTTTGGACATGTAC
TGCAGCTTCTTGTCTCTGTTTTGGATTACTGGAATACCCA
TGGGCCCTCTCAAGAGTGCTGGACTTCTAGGACATTAAGA
TGATTGICAGTACATTAAACTTTTCAATCCCATTATGCAA
TCTTGTTTGTAAATGTAAACTTCTAAAAATATGGTTAATA
ACATTCAACCTGTTTATTACAACTTAAAAGGAACTTCAGT
GAATTTGTTTTTATTTTTTAACAAGATTTGTGAACTGAAT
ATCATGAACCATGTTTTGATACCCCTTTTTCACGTTGTGC
CAACGGAATAGGGTGTTTGATATTTCTTCATATGTTAAGG
AGATGCTTCAAAATGTCAATTGCTTTAAACTTAAATTACC
TCTCAAGAGACCAAGGTACATTTACCTCATTGTGTATATA
ATGTTTAATATTTGTCAGAGCATTCTCCAGGTTTGCAGTT
TTATTTCTATAAAGTATGGGTATTATGTTGCTCAGTTACT
CAAATGGTACTGTATTGTTTATATTTGTACCCCAAATAAC
ATCGTCTGTACTTTCTGTTTTCTGTATTGTATTTGTGCAG
GATTCTTTAGGCTTTATCAGTGTAATCTCTGCCTTTTAAG
ATATGTACAGAAAATGTCCATATAAATTTCCATTGAAGTC
GAATGATACTGAGAAGCCTGTAAAGAGGAGAAAAAAACAT
AAGCTGTGTTTCCCCATAAGTTTTTTTAAATTGTATATTG
TATTTGTAGTAATATTCCAAAAGAATGTAAATAGGAAATA
GAAGAGTGATGCTTATGTTAAGTCCTAACACTACAGTAGA
AGAATGGAAGCAGTGCAAATAAATTACATTTTTCCCAAGT
GCCAGTGGCATATTTTAAAATAAAGTGTATACGTTGGAAT
GAGTCATGCCATATGTAGTTGCTGTAGATGGCAACTAGAA
CCTTTGAGTTACAAGAGTCTTTAGAAGTTTTCTAACCCTG
CCTAGTGCAAGTTACAATATTATAGCGTGTTCGGGGAGTG
CCCTCCTGTCTGCAGGTGTGTCTCTGTGCCTGGGGGCTTT
TCTCCACATGCTTAGGGGTGTGGGTCTTCCATTGGGGCAT
GATGGACCTGTCTACAGGTGATCTCTGTTGCCTTTGGGTC
AGCACATTTGTTAGTCTCCTGGGGGTGAAAACTTGGCTTA
CAAGAGAACTGGAAAAATGATGAGATGTGGTCCCCAAACC
CTTGATTGACTCTGGGGAGGGGCTTTGTGAATAGGATTGC
TCTCACATTAAAGATAGTTACTTCAATTTGAAGGCTGGAT
TTAGGGATTTTTTTTTTTCCTTATAACAAAGACATCACCA
GGATATGAAGCTTTTGTTGAAAGTTGGAAAAAAAGTGAAA
TTAAAGACATTCCCAGACAAA
Human ALK5
(SEQ ID NO: 4)
MEAAVAAPRPRLLLLVLAAAAAAAAALLPGATALQCFCHL
CTKDNFTCVTDGLCFVSVTETTDKVIHNSMCIAEIDLIPR
DRPFVCAPSSKTGSVTTTYCCNQDHCNKIELPTTVKSSPG
LGPVELAAVIAGPVCFVCISLMLMVYICHNRTVIHHRVPN
EEDPSLDRPFISEGTTLKDLIYDMTTSGSGSGLPLLVQRT
IARTIVLQESIGKGRFGEVWRGKWRGEEVAVKIFSSREER
SWFREAEIYQTVMLRHENILGFIAADNKDNGTWTQLWLVS
DYHEHGSLFDYLNRYTVTVEGMIKLALSTASGLAHLHMEI
VGTQGKPAIAHRDLKSKNILVKKNGTCCIADLGLAVRHDS
ATDTIDIAPNHRVGTKRYMAPEVLDDSINMKHFESFKRAD
IYAMGLVFWEIARRCSIGGIHEDYQLPYYDLVPSDPSVEE
MRKVVCEQKLRPNIPNRWQSCEALRVMAKIMRECWYANGA
ARLTALRIKKTLSQLSQQEGIKM
Human ACVR2A
(SEQ ID NO: 5)
GTCTGGGCTTCCGAATATGTTTTATGACGGTTGATTTTAC
ACCAGGAGGTTTGTCTCCGAGGAAGACCCAGGGAACTGGA
TATCTAGCGAGAACTTCCTCCGGATTCCCCGGCGCCTCGG
GAAAATGGGAGCTGCTGCAAAGTTGGCGTTTGCCGTCTTT
CTTATCTCCTGTTCTTCAGGTGCTATACTTGGTAGATCAG
AAACTCAGGAGTGTCTTTTCTTTAATGCTAATTGGGAAAA
AGACAGAACCAATCAAACTGGTGTTGAACCGTGTTATGGT
GACAAAGATAAACGGCGGCATTGTTTTGCTACCTGGAAGA
ATATTTCTGGTTCCATTGAAATAGTGAAACAAGGTTGTTG
GCTGGATGATATCAACTGCTATGACAGGACTGATTGTGTA
GAAAAAAAAGACAGCCCTGAAGTATATTTTTGTTGCTGTG
AGGGCAATATGTGTAATGAAAAGTTTTCTTATTTTCCGGA
GATGGAAGTCACACAGCCCACTTCAAATCCAGTTACACCT
AAGCCACCCTATTACAACATCCTGCTCTATTCCTTGGTGC
CACTTATGTTAATTGCGGGGATTGTCATTTGTGCATTTTG
GGTGTACAGGCATCACAAGATGGCCTACCCTCCTGTACTT
GTTCCAACTCAAGACCCAGGACCACCCCCACCTTCTCCAT
TACTAGGTTTGAAACCACTGCAGTTATTAGAAGTGAAAGC
AAGGGGAAGATTTGGTTGTGTCTGGAAAGCCCAGTTGCTT
AACGAATATGTGGCTGTCAAAATATTTCCAATACAGGACA
AACAGTCATGGCAAAATGAATACGAAGTCTACAGTTTGCC
TGGAATGAAGCATGAGAACATATTACAGTTCATTGGTGCA
GAAAAACGAGGCACCAGTGTTGATGTGGATCTTTGGCTGA
TCACAGCATTTCATGAAAAGGGTTCACTATCAGACTTTCT
TAAGGCTAATGTGGTCTCTTGGAATGAACTGTGTCATATT
GCAGAAACCATGGCTAGAGGATTGGCATATTTACATGAGG
ATATACCTGGCCTAAAAGATGGCCACAAACCTGCCATATC
TCACAGGGACATCAAAAGTAAAAATGTGCTGTTGAAAAAC
AACCTGACAGCTTGCATTGCTGACTTTGGGTTGGCCTTAA
AATTTGAGGCTGGCAAGTCTGCAGGCGATACCCATGGACA
GGTTGGTACCCGGAGGTACATGGCTCCAGAGGTATTAGAG
GGTGCTATAAACTTCCAAAGGGATGCATTTTTGAGGATAG
ATATGTATGCCATGGGATTAGTCCTATGGGAACTGGCTTC
TCGCTGTACTGCTGCAGATGGACCTGTAGATGAATACATG
TTGCCATTTGAGGAGGAAATTGGCCAGCATCCATCTCTTG
AAGACATGCAGGAAGTTGTTGTGCATAAAAAAAAGAGGCC
TGTTTTAAGAGATTATTGGCAGAAACATGCTGGAATGGCA
ATGCTCTGTGAAACCATTGAAGAATGTTGGGATCACGACG
CAGAAGCCAGGTTATCAGCTGGATGTGTAGGTGAAAGAAT
TACCCAGATGCAGAGACTAACAAATATTATTACCACAGAG
GACATTGTAACAGTGGTCACAATGGTGACAAATGTTGACT
TTCCTCCCAAAGAATCTAGTCTATGATGGTTGCGCCATCT
GTGCACACTAAGAAATGGGACTCTGAACTGGAGCTGCTAA
GCTAAAGAAACTGCTTACAGTTTATTTTCTGTGTAAAATG
AGTAGGATGTCTCTTGGAAATGTTAAGAAAGAAGACCCTT
TGTTGAAAAATGTTGCTCTGGGAGACTTACTGCATTGCCG
ACAGCACAGATGTGAAGGACATGAGACTAAGAGAAACCTT
GCAAACTCTATAAAGAAACTTTTGAAAAAGTGTACATGAA
GAATGTAGCCCTCTCCAAATCAAGGATCTTTTGGACCTGG
CTAATGGAGTGTTTGAAAACTGACATCAGATTTCTTAATG
TCTGTCAGAAGACACTAATTCCTTAAATGAACTACTGCTA
TTTTTTTTAAATCAAAAACTTTTCATTTCAGATTTTAAAA
AGGGTAACTTGTTTTTATTGCATTTGCTGTTGTTTCTATA
AATGACTATTGTAATGCCAATATGACACAGCTTGTGAATG
TTTAGTGTGCTGCTGTTCTGTGTACATAAAGTCATCAAAG
TGGGGTACAGTAAAGAGGCTTCCAAGCATTACTTTAACCT
CCCTCAACAAGGTATACCTCAGTTCCACGGTTGCTAAATT
ATAAAATTGAAAACACTAACAAAATTTGAATAATAAATCG
ATCCATGTTTTGTAACAAATTCACTGTGTTATTTAAGGAA
AAAAAGGTAAGCTATGCTTAGTGCCAACAATAAGTGGCCA
TTCGTAAAGCAGTGTTTTAGCATTTCTTGTGCTGGCTTGT
AATGTAGGGAAAAAAAGTGCTGTTTTTTGAAAAGATGGTG
TCATTTCCCCCTTCTTCCCATGTTTTAAAGCCCCATCTTA
TATCCAGTTCCCAAAATTTGCATACTTACCTAAGTATTTT
TTTTAGGTGTGCTGTGTTTGGGGAATATTTGAAAATTTAA
AGCATGATTTAAAATTTTTTAAAGTGAGCTGTGACACTGG
AAAGCTCTTCATTTTATCTTTTAAAATAGAGTTTTTTCTA
TTTATATATGTAAAATTGTAGTGTATTTCTTTTCACCAAA
CAGTGTGTGGGACATTCTTTATCACTGTTTTAGGATCACC
TCAGGAAGTGTCGTTACCCAGAATTCCCCACTGTCTGCTA
TGAGACTTGTAACTTTATCACTATACTTCTGCTTGGTGCC
ATCTTGTCAGAGTAATATTTGATGTCTGTGATATGTAAAG
AATTATCCTAGGATAAAGATATTAAACTTTAAGCAGATTT
CAGATGTTACTGCTTTAAAACAAATCAGGGATAACAAATT
AAACGTATAACTTAAAATATGCAATGACATTTAGAGGTAA
CCAATGTTGATATAGGTAGCATAGCCTAGCCTCCTCCCCA
AAATTGCTTTTACAACTAACACTGATACTAATTTAGGATA
GTTCATGCCTTATCCTTGCTAAGAAAATGGAATTGATGGT
AGGCAGGTGCTAAAGTGCTTTTCAAAACAATATTACGTTA
GAATACAATTGGATTCTTCCTCAAATTTATACAGGCCAAA
AAGTAAAACATTAATTTTCTGAATTTCCAGATTACCAATC
AATTAATCAACAAATAGCCAGTATTATGCTGTGTATTTCT
GTCAGGTCATTTTAAAATCCATGTTAATTTTATAAAAGAA
TTTTTTACATGTCACTGTCAGGAGCTCACTGTGAATGTGT
TGTCTTCAAATGGTTATTTAACCACACAGTACACTACATT
TTACATATATGTACGTAATCTCTGGGAATAGTAAATTAAT
TATGTTATTTATAAACAATACATAGGTCAACAGACTTTAA
GCAGGGAGGAAAAGAAGAGTAATAGCGTCTGTGTGCTGCA
GACCATTCAGAACTGTCACGTGTGTCCCCATGGTCTCATT
CATTGTATTCCTAGCAATTCCCTTTTCAATGTTGAGTTCA
CCTCTTTATTICACAAAGTACTTGGTCTCTCAATTTCTTG
ATCTGGTTTTGCTTCCATTTAAAAACTAATCAAGAAGGGA
AAATATTGAGAATGTGCATACAAGAAAATCATTAATTTCC
TGAAGATGAATTTCTACCTGTTGTGAACATTTAACTTTCT
TTTTAAAAGTTAAACAAAAATAAACAAGGGATATTATGAT
GAATGITTGGCTTATGTGAGTACTAGAGATAAAATTTTTA
AACCCAGTTATTCACAATATAAAATGTTTTCAAGTTAGAA
AAAATTTTTAGAAATCCTGGGTATTGTATTTAACTGTAGC
TAACCAATTTTAAAACTTGTATTCTTTTGAGAACTATTAT
TAATAGAAAAACTTTTTATAAGCAGTAAAATAAGAATGTT
CCAGTGACTACCTGTCCTTATACCTAGTCTTGTTAAAACT
TTCTTTTGCAGGGTATTTAGTGTTTGGTTTACAGTCAGTG
CAGAGTGGGCAAGTTAACAGAAAGTTTGAGCTAGAGATAC
TGGAAAAAAAAAAGATCAAAGAATGAGAAAAATGGTGATC
CATTTTGGGGCAAACTGAGACCCCCCAAATAACTCTTTCC
TCATGTGTATGGTGCTCCTCATGACTCGTCTTGTATTTTG
CCTTTCTGATACCCATCAGAACTGCTGCTGCTCTAACTTA
TACTCTTTACCTTGCCCAGATCTCCGCGTAAGGAATGCTT
TATGATCAACTTGCCATAGGACTGATGGATTAACCAGTGT
TCGGCTTTATTTGAAGTCTATGCCCTGCACAGCTCTTGTA
TGTATTTTAGATGCTAGAAGTTTTTTTAGCATGTGATGTG
TGATTCTTGTTTGAATTCTAGGTACCTTGTGAATTCCAGA
AAAAGAGACTGTGCTTCACGATTGTTAGTCCCATGAACTT
GCACTATCTATCTTTCATGGTGATGTTTTGAAAATACAAT
CAGGAAAAAACCCAACACCTTTGGAATTTAAAATAGAATC
ATATCATGAAATTTAAAAAGAATCTCTTCTGTTGCATTTC
CTCACCCCTAAGTAACAGCTACATTTAAGTAAAATGCAGG
TGGTAGGGGAAAAAAAACCATGGCGAGATGGTGGTTTAGT
GGAATAAACTGATTACTGGTTTTTTTGTTTTTTTTTTTTT
TTTTAAAGAAAGAAGCTTCATCACAGATACTTTCCAGTTT
CTCTTTTATACTTTTTTGAAAGATTACTTTTTAGGAACAT
TTGGTATGATATGCATAAAATTATTTATCCATTTATGGGC
AAAATGATACAAGTAGCATCTTGATTGAACATCATTTACC
TCAGATATTCAACCAGCAGTACGTTTTTTATGCAGTCTCA
ACCCATATCCCATTTGTTACCTCTCAGAATATTGGTAAGC
AGITATTTTCGCTTTACTCTGTATTTCTTGTGTTTTGGGC
ACAGGTTATTGTACTACTGTCAAATCGTACTTGCTATTTT
TTCTGCAAGTATTTAACAGAAAGCTTAAAATCCCCATAAA
ACCCCACCTTGGATAAGTGATTGTTAAATATTGTACAAAT
AAAATGTATGCTATCCCCATTCCATCCCCAAGTTAAATAA
AAAAATGAATACGG
Human ACVR2A
(SEQ ID NO: 6)
MGAAAKLAFAVFLISCSSGAILGRSETQECLFFNANWEKD
RTNQTGVEPCYGDKDKRRHCFATWKNISGSIEIVKQGCWL
DDINCYDRTDCVEKKDSPEVYFCCCEGNMCNEKFSYFPEM
EVTQPTSNPVTPKPPYYNILLYSLVPLMLIAGIVICAFWV
YRHHKMAYPPVLVPTQDPGPPPPSPLLGLKPLQLLEVKAR
GRFGCVWKAQLLNEYVAVKIFPIQDKQSWQNEYEVYSLPG
MKHENILQFIGAEKRGTSVDVDLWLITAFHEKGSLSDFLK
ANVVSWNELCHIAETMARGLAYLHEDIPGLKDGHKPAISH
RDIKSKNVLLKNNLTACIADFGLALKFEAGKSAGDTHGQV
GTRRYMAPEVLEGAINFORDAFLRIDMYAMGLVLWELASR
CTAADGPVDEYMLPFEEEIGQHPSLEDMQEVVVHKKKRPV
LRDYWQKHAGMAMLCETIEECWDHDAEARLSAGCVGERIT
QMQRLINIITTEDIVTVVTMVINVDFPPKESSL
Human ACVR2B
(SEQ ID NO: 7)
GGCTCGGCTCCGTGGCCGCCGCTCAGGAGCCATTTTGGAC
TCGGTTCAGCTCCCCTCCCCCCCACCCCTCCCCCCGTTCA
TGGCCCCTCCGGACTCGGCCCCTGCGCCCGGGGCCCGGGC
CCAGCCCCGCCGCGCTATGCCTGAGTCGGGCGCGCCCCGG
CCCGTGCCCCGCCGCCGCCCCCCGGCCCCCGCGTCGCCCC
GGAGCCCGGGCCGCAGCCTGCGCCGCCCGCAGCGGCCCTG
AGCCCGGCCCCGCCGACCGGCCCTTGGAGCCCGAACGCTG
CTCGGGGACGAAGGCGCAGGAAGCGCGCAGGGAACGAGAC
CGAAGGAAGGAGCGGGAAGGAGAGCGCAGCCGCCGCCTGG
CCCTGCGCGCCCCGGGAGCGCCGTGCGGCCCTGCCCGCGG
GCTCCGGGTGTGCGCGGGGCGGCGCCGCGGAACATGACGG
CGCCCTGGGTGGCCCTCGCCCTCCTCTGGGGATCGCTGTG
CGCCGGCTCTGGGCGTGGGGAGGCTGAGACACGGGAGTGC
ATCTACTACAACGCCAACTGGGAGCTGGAGCGCACCAACC
AGAGCGGCCTGGAGCGCTGCGAAGGCGAGCAGGACAAGCG
GCTGCACTGCTACGCCTCCTGGCGCAACAGCTCTGGCACC
ATCGAGCTCGTGAAGAAGGGCTGCTGGCTAGATGACTTCA
ACTGCTACGATAGGCAGGAGTGTGTGGCCACTGAGGAGAA
CCCCCAGGTGTACTTCTGCTGCTGTGAAGGCAACTTCTGC
AACGAACGCTTCACTCATTTGCCAGAGGCTGGGGGCCCGG
AAGTCACGTACGAGCCACCCCCGACAGCCCCCACCCTGCT
CACGGTGCTGGCCTACTCACTGCTGCCCATCGGGGGCCTT
TCCCTCATCGTCCTGCTGGCCTTTTGGATGTACCGGCATC
GCAAGCCCCCCTACGGTCATGTGGACATCCATGAGGACCC
TGGGCCTCCACCACCATCCCCTCTGGTGGGCCTGAAGCCA
CTGCAGCTGCTGGAGATCAAGGCTCGGGGGCGCTTTGGCT
GTGTCTGGAAGGCCCAGCTCATGAATGACTTTGTAGCTGT
CAAGATCTTCCCACTCCAGGACAAGCAGTCGTGGCAGAGT
GAACGGGAGATCTTCAGCACACCTGGCATGAAGCACGAGA
ACCTGCTACAGTTCATTGCTGCCGAGAAGCGAGGCTCCAA
CCTCGAAGTAGAGCTGTGGCTCATCACGGCCTTCCATGAC
AAGGGCTCCCTCACGGATTACCTCAAGGGGAACATCATCA
CATGGAACGAACTGTGTCATGTAGCAGAGACGATGTCACG
AGGCCTCTCATACCTGCATGAGGATGTGCCCTGGTGCCGT
GGCGAGGGCCACAAGCCGTCTATTGCCCACAGGGACTTTA
AAAGTAAGAATGTATTGCTGAAGAGCGACCTCACAGCCGT
GCTGGCTGACTTTGGCTTGGCTGTTCGATTTGAGCCAGGG
AAACCTCCAGGGGACACCCACGGACAGGTAGGCACGAGAC
GGTACATGGCTCCTGAGGTGCTCGAGGGAGCCATCAACTT
CCAGAGAGATGCCTTCCTGCGCATTGACATGTATGCCATG
GGGTTGGTGCTGTGGGAGCTTGTGTCTCGCTGCAAGGCTG
CAGACGGACCCGTGGATGAGTACATGCTGCCCTTTGAGGA
AGAGATTGGCCAGCACCCTTCGTTGGAGGAGCTGCAGGAG
GTGGTGGTGCACAAGAAGATGAGGCCCACCATTAAAGATC
ACTGGTTGAAACACCCGGGCCTGGCCCAGCTTTGTGTGAC
CATCGAGGAGTGCTGGGACCATGATGCAGAGGCTCGCTTG
TCCGCGGGCTGTGTGGAGGAGCGGGTGTCCCTGATTCGGA
GGTCGGTCAACGGCACTACCTCGGACTGTCTCGTTTCCCT
GGTGACCTCTGTCACCAATGTGGACCTGCCCCCTAAAGAG
TCAAGCATCTAAGCCCAGGACATGAGTGTCTGTCCAGACT
CAGTGGATCTGAAGAAAAAAGGAAAAAAAGTTGTGTTTTG
TTTTGGAAATCCCATAAAACCAACAAACACATAAAATGCA
GCTGCTATTTTACCTTGACTTTTTATTATTATTATTATAA
TTATTATAATTATTATTATTAATATTATTTTTTGGATTGG
ATCAGTTTTTACCAGCATATTGCTCTACTGTATCACAAAC
AGCGGACACGTCAGCAGGCGTTGAGGTGCTGAGCTGTGGA
TGCAGAACCAGCGCCATGCTGAAGAGCCTCAGCCACCTCC
TGTCCTTTGGGATTCGTTTTTCCCGCTTTCTCTTTGTTTG
TCGTCTCAGAATCTGTGACACAAAGAAACCCATCTCCTGT
CTTAGGAAACCTAATGCTGCAAACTCTACCTAGAGGAACC
TTTGAAGACTGTTACATAAGAACATACCTTCCTCAGAAGA
GGAGTTTCCTCTGCCCTCTGCCCTTCTCCCCTGCCTCCCT
CCCTCCCCTCCTTTTATTTTGTTTTAGTGAGCTTAAGAAA
CAGCAGATGTGTCTTTCACGGATCTAACGGGTGTTGTCCT
GATCGAGAAAAAAACTGGGATGAGAATGGTTTGGACTGGA
GTTGGAAGGGGAGGACGGTACTGGGGGTAGGGTTTGGAAC
AGAGCTACACTGGACTCGGGCACATTCGGAGCAGCATCCT
TTAGTATGGAGGCTACTTCTCAGGTAACCAGGAATTGAGG
GGAAGGACCTTGTGGAGGCCGAGCATTAACAGCAAGAGCG
GGGTTTGGAGAAAGTCTGAGATTGGGTGCAGCCCTGACTT
ACCTGCTGGCCCTGACCAGTTTCTTTTCACTAACTTGGCC
TTGGGCATAGGATGAAACATTTTTTCTGCCCTAATTTTAA
AACTAGGTGAGGGTAGAATCATCACAGGTTAGGAATACAT
TCTTCATAAGACACGATGCTGTAAATACCCTTAATGGACG
AAAAGTTGAAATACTTTTGTTTCCTCTTGGAGCAGTTCAG
GGAAATGCCCACAGGGGATTGTCCTGCACAGATAGGGCAA
GAGGATTTCCTGGGTGGAGTCTGCCAAGGCCTGCCTCGCT
GGGGACCCCAGAGTCCTGCACCTCTGGTTCCGCCCCAGGT
GGTGACATTACTGTCCCCGTTCTGTGGCTCGTGGACAAGA
CTTTCTCCAGACCCCTTAAAGTGGTACATATTCTAAAAAA
CTGTTTTTCTATTATGCCATAACCTTGCTCTAGTCAGTGA
ATGTTCCTAATGCTGCTGTTTCAACATTTGAATTCTTTTT
AATTTATGAAACATGCTAAATTTTTTTTTTCAAACAAAAC
ACACACATCCACATATACACATGCTTCGCTATGTGGCTTC
CAAGGITTAAATTTTGAAAAGTAAAAGAATTAAAACTTCA
CGACCACAGATCACCTCAAACCAGAAATACCTCAGAATTT
TCTACTTATGTAAGGTTTATTATATATTTTGTTAGTTGTG
TTGTCTTGTAGTAAGTATATTTTAATGTAAGTTGGCTTTT
GTGACAAGGAAGTTTAAAAGAAATAGAGAAAAAGAAAAAA
GTTTGCATCTTCTAGGGAGTGCTACCATTTTTGTTTGATA
ACGCCCCCTTGTAAATAATTGTCATCAACTGTAGGTTGGC
TGTCIGGGCCAAGTCTGGGCATTTATCAGTCTTGTTTGTG
AAGGCTTTTCCTTCTGGITTCTTTAGATCATTTTATTTAA
AAACAGTGCATCTCTTCATCGTGAGGGTAGGCAAGGCGGG
GGCCGTGGGGAGAGGTTGACCTGGGTGAGAACTGAAGAGG
CCGCCTCCTCTTGGGTTGTTTGGAGCTTCACATGTAATTC
ACATGTAACATGTAACTTGATCGGTCAGTGTTCAGAATGA
CAAGTAACCCCGCTTAAACTTGGTAGAAGGATGGCCCTTA
GACCTGAATGGGGTGATTTTACTTGGGATTTAACTTCTTC
AGCAAATTAACAGCAACGTTGGAAGAGATCTGTGGCGCCT
CIGTGAAGCACACCGTGACTCAGGCCAGTCTTTTAGTGCA
GCGTGTCTGGGAGTGAAGGGTTTTGCCCTTGCTGGTCTTG
GAGTCCACAGTGTGAGGGGCACTGCACATGCCTGGGCATC
TACCTAGTGTGCTATGTTCAGTGTCTGGGGCTTACTGCCC
CGGGGTCCTTTCCTCTGGGTGTTGGGGCACAGGGTGCTAT
GGGAGGCCCATTTGCTTCCCTCTCGGAGCTCAGTTTTTGC
TTCATGGGTCAAAATGTGGGCTGGCCAAGTGGTTACAGGA
ACAGGGTTTCGGTAAGCTATGTTGTCTTTTTTTTTTTTTT
TTTTTTTTTTTTTAATGGTTTGATTTTGTGCTGTGGTATT
TTTTTTCCCTTAGAATAATTTTTAATGGCAAAACAGGCCT
TACAGCAGTTGCTTTTCTTTACCATTTATTTCTTTAAGAA
GCTTTAAAATATTTATTGAAAAGTGCCATATCTAATTTCT
TTAGCTTTCGCCTCAGGCAGTGCAGGCATCTTTACTTTTC
ATCCTCAGAAGAAACAAACGACTAACAAATGTAGCAAATT
TACTGCAGGAATAGTTAGGTCATGATACTACCTGAACACT
AAACCCCAGCCTCTTTGTTTGGTTTTAGTTCCTCTGGGTG
GTTTTTCTTTTGTGTGCTGGCTTGATTCTTGTGAGAAGTT
TTGACCTGGCCAAGGGAGGGTTGAGCCATGGTTCTGGTGT
GGGACTTTGCGGTCAAGACACAGTACAGACAGGTCAGGCC
TGCGTGCCTTTTCTCTGGGTGGCCTCCCCGTTAGGCCCAC
CGTACGCTCAGCCACTATAGTGTCCCTGTGGGGCCTTGCC
ATCAGATTGTGTGTCAGGAGATGGTACCTTTTTGGTGTGG
CTGGGGAGGAGTGTGGTCCATGCCAGTTCTTTGGGCTTCA
GGCCACTCTTCCCCTCATGCTGTGGTGTAAAGTGCACCCA
TCAGGTGGTATATCTGGTTCTGATGGCAAGAAGAAGGIGG
GGGATCTCCTTATAGGGCATGGGTCTAGGAGCACAGATGG
GCCTTTTGCCCCGGGTAAATGCTTGTCTGTTTGCTGTCAT
GTGTTCTTTGAGGAGTGAGCCATCTCGAGCCCTGCTTTGA
ATTTACTGGGTCATAGAGCCTCTGCCTGTGCTCTTTTCCA
TAATGACTTCATGTGACATGCACTTTTGGTGGGCTCAGAT
AATTGGTTTCTTTTTGTTTTTGACCTCAGGCTCTGTGGCA
GACTGGGGAAAATGGGGCCTGGCATCATTTTCCCTGTCAA
TGGGAGGGGCTGTTCCATGCAGGGTGGGAGGGGACCAAGT
TAGCAGAGAGTAGCCAAGGATCCTTGCTTCTTCCTTTCTA
GTGTGCTGTCATCCAAGCAGGCTCCTGGCTGTAGGGATGG
GCCTTGGGGAAGAATCTTCTTTGAAAGCATCTATGATAAC
TGAGAAGTCATCCCTAGTTGGAGAAATCCAGTAATGAGCA
GAAGGAGGAAGCAAGTGAGGACAGAGGCCATTGTATTACA
GTGTCACGCAGAGGGCCCTCAATGATGGGGCATTGGGGAA
GGCTGTAGACATAGTCATCAGAACATCCTGGCCTGGCATA
AGCTGGGTTTTCTCCTGGGACCATTGGTCCTCAGCAGGAG
TTCTTTGCATGAGTTGCTCAGGGGCAAGGGCTGCAAGTGG
GCTGTGCTTAGGAGAAAGTGACACCTGGCAGTGAGGGAAG
ATGGTGAGCATTATTAGCCTTTGTTGTCCAGCATGGCCTT
CTTGTCCTGTCTGCTCTGGAGAGGAGCCTGTGGGACCAGT
CCTGCCTGGGGAGGGCATACCCACACGTGCCAGCTGATTC
TGACTCTGAATACATCATGTCCGGACTTGGGGGTGITTCT
GCAGAAAAAGGAGGTTGTTTTTCAGCCTTGAACATCTTCA
GGAGGATAGAGACTCTTGCTCACATATTCTTAGCAAAGGG
AAGGGTCTCTCATCTCCAGGCCACAGAGATAGTTCTTCCA
TTGCCCTAAGAGGCTAGGCTAACCCTCTTGACATAACTTA
GACAGCAAAGCACTTCATCCTGTAGTTGGGCTCTGTCACC
TTTCTCTTCAGTTGGCCACATTCTCGITTCCTCCATCCTG
CTATGCTTTGTGTGCTCGGGCTGTGTGTGGGGTTTTTCCC
TGGTGGAAGGAAGCCCAGCTGTGTATTGAATGTCCTTCAT
GTGTTGTGTGTGGCTCAGAAAGCCTGTCACTTGGCCCCTG
TGCTCTGAGCCGTGAGGGTGGGGAGGTGGCTGTTCCATTA
AAGTGGGAGTATTGGATGGCCCTCTTGAAACTAGAATTTT
GCCTTTTTTAGTATGCAGTATAAAGTTTCCAGCATCTATT
GGTAACACAAAGATTTGCTGGTTTTTAAAATAATACAGTA
AGCATAAGTATGTAAGTTTTTAGAATTGGTACTAGAAGTT
GGACAGCTAGTTATTCTCGAGAACTTTATTTCACTAGAAA
AATATACTAATTGGAAAGCAGTTTCCAGGAGTTAACTCAG
TTTAATTTTCAGTCTCAGTTATTTTAGCCTGTTGAGTTTT
TGATGGCACACCTTTGGAGAGATGGCCACGCCTGATTCCC
ATTTCAGGGGCATCAGACCATACCTTTTTAAGAAGCTCCG
TGAATCTAGTCATCTACCCTTCATCCTGGGCGAACAGCCA
AAAAGAGAAGGGGACAAGGTGTCTTTTTCTCCTTCTCACT
GGGGTGACATGAATTCTTTTAGTTAATGGCTGTTTGCAAA
TTCTAAACTAATGAAATACTTAGCAGCTAACATGTTCAAT
CTAGTAATGATGAGTTTAAATCTCAATTGACAGTAATGTT
TTAGATAAACAGGCCCAGTAATTCAGTTGATGAACTGTAT
ATCTTCTCAGTCTAGATTTGTAAATGTTTAATGAATTCAG
GGTTATAAGCATAGTTCTTTAAGTAAGATTCCAGATAGTT
GATTTGCAACCAGCAGTCTACCTATGAATGTATCCCAAAC
CTTTAGAAGATTGGAAAAGATTTTTGAAATAATGATTTAG
TTTTGTAGGAAAAACACCCCCTTGAAAATTAATTCGGTTG
ACCCAGTAACATTTTTTAAAACAATTGGTGGCTCCAAAAG
GCCTGCCAACAAAGAAAAGTCCAAATTATCTAGTGGGACA
TTTTGAATGTTTTATGTTTATTTTGGGTCCACTGTAAACT
TTGGTTCAAAAAAGAATTTGAATTTAAAGAATTTACCATT
ATTTAAATTATTACCAAGTTTTTACATTTTCATGATGGTA
TTTTCCAGGTATGAATGAAACATGACTTTTTGATTGTGGT
ACTTCCTGTATCCCCTGTAGTGCCAAAACCAGTGATACTT
TATTTGCTCCTATGGCAGCTCATAGAGGTAACCGAAGTGA
TTTTTCCTCAGTAATTGAAACACATATTCTCTAAATGCCA
ATGTGTGGTGATGGGCCCTGCACTGCCTTCATTTCTCTAG
GGCAGTGTCTTTGGATTGTCTAGGGCCTAGGTAATTCTGA
GAACTACTGTAAACCAACCACAGGGCACTAAAGCAATGTA
CACACCACTCTTTGTGTGTATGGAAGGGGTTATATAAACC
TGGGCTATGCTGGACATCTACAGAAGAGTATTACATTCAC
TTGCAAAGITTACATTTTTGAGCTCACAGTTATGAAAAAT
ATGACCCACAAGTTTTTCAGGCAGGTGAGGATGGGTCTTC
TTGCAAATGCATGAGTTCTGTCTTGAGTCCTGGGAACTTC
TCTGTTGGTTGAGTGTGGGCTCATTCCCTGACTCTCCTAA
TCATGTTTGCGTCAGAATGTTAGCATTGTAAATAAAAGAA
TAGGTTGTATAATAGATACACAACACTTGAAACTTTACTT
TAAAAAAATCGATAGTTCTACATATATATTTAGTTATATC
ACTTGACAGATTTCTTCTACACAGTGTGGAGATTGTTTTA
TACCACAGATTATTTTTATAAAGTTAGTGAATTTGAATGA
TTTTGTAATCAGAGCTAATGAGCTTTACCTTTCAAGAGAA
ACGTACACTGGAGCATGAGTGGTGTGGAACTTTTACTTAG
TGTTTATATGGATTCTTGTGATACACTGGCAGACTGGAGT
CAATTTGCGGGTCTTTTTTGGCCAAAACTCCACTTGTGGT
TGTGTAGGACAGTGATATTCAGCTCAGCTTCTTGTGGATT
GGGAGGAGAGAGGGCCTGCAATGTGTTTTACATTGGTGCT
TCCTCCTGAGATTTCTGTTGAACAAAGGGTTCTGAGGTCA
AAAATTAGTTTGTAAGCCTTTGCCATAGGACATAGTCATG
TGAGAGTGTTTGGGGGAACAGAAATTGTATAGGGGTGCCT
ATTGGGGTGGGATGGGACTCGAATAAGATTCAGGTACAAA
AACTTTGAAATGAGAATCTGGIGGITTGAGTAATCCACCA
GACTGAATTATCTAAGATCACATTATCCAGGTTGGGGGGC
AGAATTACCCAGTTAAGTAATTGTTCAGAAAAGTGGGGAG
GGTGGCATGTGGATGCAGTGATCCAATTAAATGGAGAGCT
GCCAGGCACATTTTGTCCTCTCTGGTCAGTGAGAATGGTT
GGGTTGGCTCGCTGCTTCAATCTGTGGAATCAGCCAGGAG
CCCAGTGAGGAAGCTCAGAACCCCAGTAACAGCAGAGCAT
CTTTCAGATAGCTCCAGAGTTTTCCTGCTTTTCTGAGGAA
GCTCAGCATCACTGCCACAATACGGAAAGTGGTCTTCATT
TTAGCCTATTTATTTTTAGGCAGAGAGTGGATGGTTATTT
GTGTGGGACTTTTGGTGGCGATATATAATGAATAATTAAG
TTAATTTCTGGTATGCATAATGGCCAGTCCTGAGGCCCAG
CTGAAGACCTGTCCCCCAGACCCTGCCCGCTGGCTTCAGG
CTGCTGCTTCTAGACAGAGGTGCACTGGACGGGATAGTTT
TATCAAGAGAATCCCTAATGTGTCATTTTAAACCAGCTGT
GCTTTTTATTCATTCTGGTTGAGCGTATAGGTTTACACTT
TACCCTTTTTATACTTGGAATAAATTTAGITCCAGCAGAT
CTAGTAGCACTCCAGAAACCAACCCCATCTGTTCCCCATA
AAAAGAACATTTTCTCTGCTCTCCAGCCACGTGTCTTGGA
ATGTAATTCTGTTGTGCCTTTGTTTTTATCACTCTCTTCG
CCCCAAAAGCAACTGCTGTAAGCTTTTTTCTACTTGTCTT
TTCTAGTCCCCAACCTCTACCTTTTTCCTTTTTCCCAGCC
CTAATTTCTGGATGCACTTCTGTGATCCAGGTATTTTAAG
AACCAGTTACCTCAGACCTCATGTTGAACAGTGTCGCCAT
CTGGGTCCTCTTGATACTGCAGACTTTTAACGTACACATG
CAGGAACCCTGCTGAGCGTGGGCACTTGTTTTAAAGCAAA
ACTCTTCCCAAGGACTGAAGAAAGGGCTTCTGGCAAGCTC
GTCATGGCATTGTGGTGGGATGGGTCTAGAGTGTCATCTG
AATGGTGCTTCCTGTGTTCCTCTTTGAATTCTGCCATTTT
CAGTATTCTTGTGTGTCTGAATAGGCAAAGCGATTTAATT
GGCTGGTCTTGCACGCAAATTAGTTCCAAAGATAAGCTCT
TTGTAACACATTTCCAGTCGCTAATGCTCAAATGTAGAAC
ATTCCTTTAAATGGCAGGATAAAAAACCCACTATCCACCA
TAGTGCATTTTGGGAAGATGTCTGTAGCATATGTTGCTGT
GAAATTAGGCCTTGTGGGATATGGCTGTTTGTCATTTTGA
TGTATTTTAAATAAATATATATATTTTTTAAAGAGCCTTT
TTTACCAGTTCAAAAAGTTTAATTAACCAGCAGTCACCGC
ATCTGAATTTTTGTCTCTGGGGCATAGATGGCAGACCAAG
ATTAAAAGTGGTAACTCAGCTATACGAGCATGGGCTACCT
TCCTGGGCTCTCCTGCAGTCCTGTAGACCTGCTGTTCCGC
AGACCATGGGACACAAGGTCAGTGTGTTCCCAGTGAGGGT
CCCAAGTCAGTCATCTTAAGTGTTTGTTCTCTGCCCCATT
CAGTGGACTGTTGACTTCAGTCCCTGCAAGTGCTTTAGCC
CGAGTGGGGTTTTCTCAGAGCACTGCCACGAGTTAAGTGT
GTGTTTAGCCAAATAATTTCTCCGTAAGGGAAAAATGCAG
TCACCCAAATTTTACCAACAATGACAGAGATGAGAGTAGA
AAAGATTAGGCAACATCTGAGTTTTAACTTGAAAAGTGTC
CAAGTCATCATGAAAGGCCGACTGGGAGCAAGTGATTATT
AGAGATTCTTCAGGAGACCTCATCTGAAAATGTTAAGACT
GCCAGTGAGGGAAGGAATTGTTAAAATGCCAGCGGCTTTT
TTTTCCTCTTTTTTTCTGTAATTCTGTAAAAATGCAGAGA
AAGTTGAGTGGTACTTCAGAATTGAGGGAGAGGGTTACCG
CAGAGTAGAAATATATTTCTAGATTTCAGTTCCACACCAC
AAATCCACAACAATGCCATTTTTCAACTGTACAAAAATCT
GCTTATGAACTGGACATGATCTTAATGGTAGTGTCAAAGG
CCAAGTTTTTCACCTGTTAATATTTTTCCACATTTGTCCT
TGAATCTGAATAACTTTATACAGTACTGTAAATTTAACTT
ACATCGAGTTTGTTGTCAATTCTTATGAAAAGAGCTTTCT
GCATGTAACACATACGGTTAAAGAACACAGCAAAGGACAA
AATTTGCAGGAACAGTTTTGGAACCAACAGAAAATGTCAC
CTTTTATTTGCCATCTTATATATATCTATCAGTTTTACCA
GCTACTTCTAAATTIGTACATTATTTGTAAGGGAAAGAAG
GAAAACCCTAAGACTTGTCTAACTTAGTGGAGAATGTGTG
TGTTGGGCTTAGGATGGATAGCTAAGTCTTATTGAGCTGT
GTTACCTAACTTGTATATAAAAATTGTAATTAAAAGTTTG
GGTTCACCTGTTTCTCACAGTTTAAAATGATGAGTAATTG
CAAACTCTGGAAATGTGACTAGTATATGATTTAAGGCTGT
AGAAGCAAGGAAGCTCTTTCAAGTGCTAAAACTAAAGACT
TCTAGTTTTTGGCTCAAATAAGTACTGTTTGTATACCAGG
ATATGTGAGATGTAAATGTAGTAGGTCACTTTTCACCCTT
GTAGCTATAAAATAAAAATTTTGTAGAACAGAAATAGCTT
GTACTACTGAATTAACAAAAGTTATACTAAAGTATCATGT
TTAAAAAAAATATATATATATATACAGAGTTAAGCTTGTT
GCTGTTACCCTGTCTGGATTTGAAAAGTGTGCTGATTTAT
ATATATATATTACACACACACACACACACACACACACACA
CACACACACACACACACACACACACACACACACACACACA
CACATACACCTAAAATGGCCTAAAGCAGACATCCATGTAA
TTACAGTTGCAAAATGAAAACATTTTGGAAAGAACATTGT
ATCATAGTTCATTCATTTGCAGTGGATCTTTGTTCCTTTT
TACTGTGGTAATTTTAGAAATGAGTGTCAAGTTTGAAATT
AGATCTGCTAAGTTGGGGTTTTGCTGCTTGAACTCTGCAC
TGGGTCCTCAAATAAACCGATGTGAATGTAGTTTTTTCCC
CCTGTGTGAAGAAGCAGTTACACCCCAACAATAGGAGGAA
AAATCTAGAACTATTTCAAGTTTTATCTTTTTGTATATGA
AAATAAAATAATAATAAAACAA
Human ACVR2B
(SEQ ID NO: 8)
MTAPWVALALLWGSLCAGSGRGEAETRECIYYNANWELER
INQSGLERCEGEQDKRLHCYASWRNSSGTIELVKKGCWLD
DENCYDRQECVATEENPQVYFCCCEGNFCNERFTHLPEAG
GPEVTYEPPPTAPTLLTVLAYSLLPIGGLSLIVLLAFWMY
RHRKPPYGHVDIHEDPGPPPPSPLVGLKPLQLLEIKARGR
FGCVWKAQLMNDFVAVKIFPLQDKQSWQSEREIFSTPGMK
HENLLQFIAAEKRGSNLEVELWLITAFHDKGSLTDYLKGN
IITWNELCHVAETMSRGLSYLHEDVPWCRGEGHKPSIAHR
DFKSKNVLLKSDLTAVLADFGLAVRFEPGKPPGDTHGQVG
TRRYMAPEVLEGAINFORDAFLRIDMYAMGLVLWELVSRC
KAADGPVDEYMLPFEEEIGQHPSLEELQEVVVHKKMRPTI
KDHWLKHPGLAQLCVTIEECWDHDAEARLSAGCVEERVSL
IRRSVNGTTSDCLVSLVTSVINVDLPPKESSI
Human TGFBR2
(SEQ ID NO: 9)
GGAGAGGGAGAAGGCTCTCGGGCGGAGAGAGGTCCTGCCC
AGCTGTTGGCGAGGAGTTTCCTGTTTCCCCCGCAGCGCTG
AGTTGAAGTTGAGTGAGTCACTCGCGCGCACGGAGCGACG
ACACCCCCGCGCGTGCACCCGCTCGGGACAGGAGCCGGAC
TCCTGTGCAGCTTCCCTCGGCCGCCGGGGGCCTCCCCGCG
CCTCGCCGGCCTCCAGGCCCCCTCCTGGCTGGCGAGCGGG
CGCCACATCTGGCCCGCACATCTGCGCTGCCGGCCCGGCG
CGGGGTCCGGAGAGGGCGCGGCGCGGAGGCGCAGCCAGGG
GTCCGGGAAGGCGCCGTCCGCTGCGCTGGGGGCTCGGTCT
ATGACGAGCAGCGGGGTCTGCCATGGGTCGGGGGCTGCTC
AGGGGCCTGTGGCCGCTGCACATCGTCCTGTGGACGCGTA
TCGCCAGCACGATCCCACCGCACGTTCAGAAGTCGGATGT
GGAAATGGAGGCCCAGAAAGATGAAATCATCTGCCCCAGC
TGTAATAGGACTGCCCATCCACTGAGACATATTAATAACG
ACATGATAGTCACTGACAACAACGGTGCAGTCAAGTTTCC
ACAACTGTGTAAATTTTGTGATGTGAGATTTTCCACCTGT
GACAACCAGAAATCCTGCATGAGCAACTGCAGCATCACCT
CCATCTGTGAGAAGCCACAGGAAGTCTGTGTGGCTGTATG
GAGAAAGAATGACGAGAACATAACACTAGAGACAGTTTGC
CATGACCCCAAGCTCCCCTACCATGACTTTATTCTGGAAG
ATGCTGCTTCTCCAAAGTGCATTATGAAGGAAAAAAAAAA
GCCTGGTGAGACTTTCTTCATGTGTTCCTGTAGCTCTGAT
GAGTGCAATGACAACATCATCTTCTCAGAAGAATATAACA
CCAGCAATCCTGACTTGTTGCTAGTCATATTTCAAGTGAC
AGGCATCAGCCTCCTGCCACCACTGGGAGTTGCCATATCT
GTCATCATCATCTTCTACTGCTACCGCGTTAACCGGCAGC
AGAAGCTGAGTTCAACCTGGGAAACCGGCAAGACGCGGAA
GCTCATGGAGTTCAGCGAGCACTGTGCCATCATCCTGGAA
GATGACCGCTCTGACATCAGCTCCACGTGTGCCAACAACA
TCAACCACAACACAGAGCTGCTGCCCATTGAGCTGGACAC
CCTGGTGGGGAAAGGTCGCTTTGCTGAGGTCTATAAGGCC
AAGCTGAAGCAGAACACTTCAGAGCAGTTTGAGACAGTGG
CAGTCAAGATCTTTCCCTATGAGGAGTATGCCTCTTGGAA
GACAGAGAAGGACATCTTCTCAGACATCAATCTGAAGCAT
GAGAACATACTCCAGTTCCTGACGGCTGAGGAGCGGAAGA
CGGAGTTGGGGAAACAATACTGGCTGATCACCGCCTTCCA
CGCCAAGGGCAACCTACAGGAGTACCTGACGCGGCATGTC
ATCAGCTGGGAGGACCTGCGCAAGCTGGGCAGCTCCCTCG
CCCGGGGGATTGCTCACCTCCACAGTGATCACACTCCATG
TGGGAGGCCCAAGATGCCCATCGTGCACAGGGACCTCAAG
AGCTCCAATATCCTCGTGAAGAACGACCTAACCTGCTGCC
TGTGTGACTTTGGGCTTTCCCTGCGTCTGGACCCTACTCT
GTCTGTGGATGACCTGGCTAACAGTGGGCAGGTGGGAACT
GCAAGATACATGGCTCCAGAAGTCCTAGAATCCAGGATGA
ATTTGGAGAATGTTGAGTCCTTCAAGCAGACCGATGTCTA
CTCCATGGCTCTGGTGCTCTGGGAAATGACATCTCGCTGT
AATGCAGTGGGAGAAGTAAAAGATTATGAGCCTCCATTTG
GTTCCAAGGTGCGGGAGCACCCCTGTGTCGAAAGCATGAA
GGACAACGTGTTGAGAGATCGAGGGCGACCAGAAATTCCC
AGCTTCTGGCTCAACCACCAGGGCATCCAGATGGTGTGTG
AGACGTTGACTGAGTGCTGGGACCACGACCCAGAGGCCCG
TCTCACAGCCCAGTGTGTGGCAGAACGCTTCAGTGAGCTG
GAGCATCTGGACAGGCTCTCGGGGAGGAGCTGCTCGGAGG
AGAAGATTCCTGAAGACGGCTCCCTAAACACTACCAAATA
GCTCTTCTGGGGCAGGCTGGGCCATGTCCAAAGAGGCTGC
CCCTCTCACCAAAGAACAGAGGCAGCAGGAAGCTGCCCCT
GAACTGATGCTTCCTGGAAAACCAAGGGGGTCACTCCCCT
CCCTGTAAGCTGTGGGGATAAGCAGAAACAACAGCAGCAG
GGAGTGGGTGACATAGAGCATTCTATGCCTTTGACATTGT
CATAGGATAAGCTGTGTTAGCACTTCCTCAGGAAATGAGA
TTGATTTTTACAATAGCCAATAACATTTGCACTTTATTAA
TGCCTGTATATAAATATGAATAGCTATGTTTTATATATAT
ATATATATATCTATATATGTCTATAGCTCTATATATATAG
CCATACCTTGAAAAGAGACAAGGAAAAACATCAAATATTC
CCAGGAAATTGGTTTTATTGGAGAACTCCAGAACCAAGCA
GAGAAGGAAGGGACCCATGACAGCATTAGCATTTGACAAT
CACACATGCAGTGGTTCTCTGACTGTAAAACAGTGAACTT
TGCATGAGGAAAGAGGCTCCATGTCTCACAGCCAGCTATG
ACCACATTGCACTTGCTTTTGCAAAATAATCATTCCCTGC
CTAGCACTTCTCTTCTGGCCATGGAACTAAGTACAGTGGC
ACTGTTTGAGGACCAGTGTTCCCGGGGTTCCTGTGTGCCC
TTATTTCTCCTGGACTTTTCATTTAAGCTCCAAGCCCCAA
ATCTGGGGGGCTAGTTTAGAAACTCTCCCTCAACCTAGIT
TAGAAACTCTACCCCATCTTTAATACCTTGAATGTTTTGA
ACCCCACTTTTTACCTTCATGGGTTGCAGAAAAATCAGAA
CAGATGTCCCCATCCATGCGATTGCCCCACCATCTACTAA
TGAAAAATTGTTCTTTTTTTCATCTTTCCCCTGCACTTAT
GTTACTATTCTCTGCTCCCAGCCTTCATCCTTTTCTAAAA
AGGAGCAAATTCTCACTCTAGGCTTTATCGTGTTTACTTT
TTCATTACACTTGACTTGATTTTCTAGTTTTCTATACAAA
CACCAATGGGTTCCATCTTTCTGGGCTCCTGATTGCTCAA
GCACAGTTTGGCCTGATGAAGAGGATTTCAACTACACAAT
ACTATCATTGTCAGGACTATGACCTCAGGCACTCTAAACA
TATGTTTTGTTTGGTCAGCACAGCGTTTCAAAAAGTGAAG
CCACTTTATAAATATTTGGAGATTTTGCAGGAAAATCTGG
ATCCCCAGGTAAGGATAGCAGATGGTTTTCAGTTATCTCC
AGTCCACGTTCACAAAATGTGAAGGTGTGGAGACACTTAC
AAAGCTGCCTCACTTCTCACTGTAAACATTAGCTCTTTCC
ACTGCCTACCTGGACCCCAGTCTAGGAATTAAATCTGCAC
CTAACCAAGGTCCCTTGTAAGAAATGTCCATTCAAGCAGT
CATTCTCTGGGTATATAATATGATTTTGACTACCTTATCT
GGTGTTAAGATTTGAAGTTGGCCTTTTATTGGACTAAAGG
GGAACTCCTTTAAGGGTCTCAGTTAGCCCAAGTTTCTTTT
GCITATATGTTAATAGTTTTACCCTCTGCATTGGAGAGAG
GAGTGCTTTACTCCAAGAAGCTTTCCTCATGGTTACCGTT
CTCTCCATCATGCCAGCCTTCTCAACCTTTGCAGAAATTA
CTAGAGAGGATTTGAATGTGGGACACAAAGGTCCCATTTG
CAGTTAGAAAATTTGTGTCCACAAGGACAAGAACAAAGTA
TGAGCTTTAAAACTCCATAGGAAACTTGTTAATCAACAAA
GAAGTGTTAATGCTGCAAGTAATCTCTTTTTTAAAACTTT
TTGAAGCTACTTATTTTCAGCCAAATAGGAATATTAGAGA
GGGACTGGTAGTGAGAATATCAGCTCTGTTTGGATGGTGG
AAGGTCTCATTTTATTGAGATTTTTAAGATACATGCAAAG
GTTTGGAAATAGAACCTCTAGGCACCCTCCTCAGTGTGGG
TGGGCTGAGAGTTAAAGACAGTGTGGCTGCAGTAGCATAG
AGGCGCCTAGAAATTCCACTTGCACCGTAGGGCATGCTGA
TACCATCCCAATAGCTGTTGCCCATTGACCTCTAGTGGTG
AGTTTCTAGAATACTGGTCCATTCATGAGATATTCAAGAT
TCAAGAGTATTCTCACTTCTGGGTTATCAGCATAAACTGG
AATGTAGTGTCAGAGGATACTGTGGCTTGTTTTGTTTATG
TTTTTTTTTCTTATTCAAGAAAAAAGACCAAGGAATAACA
TTCTGTAGTTCCTAAAAATACTGACTTTTTTCACTACTAT
ACATAAAGGGAAAGTTTTATTCTTTTATGGAACACTTCAG
CTGTACTCATGTATTAAAATAGGAATGTGAATGCTATATA
CTCTTTTTATATCAAAAGTCTCAAGCACTTATTTTTATTC
TATGCATTGTTTGTCTTTTACATAAATAAAATGTTTATTA
GATTGAATAAAGCAAAATACTCAGGTGAGCATCCTGCCTC
CTGTTCCCATTCCTAGTAGCTAAA
Human TGFβRII
(SEQ ID NO: 10)
MGRGLLRGLWPLHIVLWTRIASTIPPHVQKSVNNDMIVTD
NNGAVKFPQLCKFCDVRFSTCDNQKSCMSNCSITSICEKP
QEVCVAVWRKNDENITLETVCHDPKLPYHDFILEDAASPK
CIMKEKKKPGETFFMCSCSSDECNDNIIFSEEYNTSNPDL
LLVIFQVTGISLIPPLGVAISVIIIFYCYRVNRQQKLSST
WETGKTRKLMEFSEHCAIILEDDRSDISSTCANNINHNTE
LLPIELDTLVGKGRFAEVYKAKLKONTSEQFETVAVKIFP
YEEYASWKTEKDIFSDINLKHENILQFLTAEERKTELGKQ
YWLITAFHAKGNLQEYLTRHVISWEDLRKLGSSLARGIAH
LHSDHTPCGRPKMPIVHRDLKSSNILVKNDLTCCLCDFGL
SLRLDPTLSVDDLANSGQVGTARYMAPEVLESRMNLENVE
SFKQTDVYSMALVLWEMTSRCNAVGEVKDYEPPFGSKVRE
HPCVESMKDNVLRDRGRPEIPSFWLNHQGIQMVCETLTEC
WDHDPEARLTAQCVAERFSELEHLDRLSGRSCSEEKIPED
GSLNTTK

All references, patents and patent applications disclosed herein are incorporated by reference with respect to the subject matter for which each is cited, which in some cases may encompass the entirety of the document.

The indefinite articles “a” and “an,” as used herein the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between and including the upper and lower ends of the range are specifically contemplated and described herein.

What is claimed is:

1. A method of increasing muscle weight, reducing body fat content, and/or improving glucose metabolism in a subject, comprising administering to the subject an agent or a combination of agents that inhibit(s) ALK4 and/or ALK5 signaling in the subject.

2. The method of claim 1, wherein the agent or combination of agents inhibit(s) ALK4 and ALK5 signaling in the subject.

3. The method of claim 1, wherein the agent or a combination of agents is/are administered to the subject in an effective amount to increase muscle weight in the subject by at least 3%, at least 4%, or at least 5% relative to a control or baseline.

4. The method of claim 1 or 3, wherein the agent or combination of agents inhibit(s) ALK4 and/or ALK5 signaling by binding to ALK4 and/or ALK5.

5. The method of any one of the preceding claims, wherein the agent or combination of agents inhibit(s) ALK4 and ALK5 signaling specifically in myofibers of the subject

6. The method of claim 5, wherein the agent or a combination of agents is/are administered to the subject in an effective amount to increase tricep muscle weight in the subject by at least 3%, at least 4%, or at least 5% relative to a control or baseline.

7. The method of claim 5 or 6, wherein the agent or a combination of agents is/are administered to the subject in an effective amount to increase quadricep muscle weight in the subject by at least 3%, at least 4%, or at least 5% relative to a control or baseline.

8. The method of any one of claims 5-7, wherein the agent or a combination of agents is/are administered to the subject in an effective amount to increase gastrocnemius/plantaris muscle weight in the subject by at least 3%, at least 4%, or at least 5% relative to a control or baseline.

9. A method of increasing muscle weight, reducing body fat content, and/or improving glucose metabolism in a subject, comprising administering to the subject an agent or a combination of agents that inhibit(s) ACVR2A and ALK5 signaling in the subject.

10. The method of claim 9, wherein the agent or a combination of agents is/are administered to the subject in an effective amount to increase muscle weight in the subject by at least 3%, at least 4%, or at least 5% relative to a control or baseline.

11. The method of claim 9 or 10, wherein the agent or combination of agents inhibit(s) ACVR2A and ALK5 signaling by binding to ACVR2A and ALK5.

12. The method of any one of the preceding claims, wherein the agent or combination of agents inhibit(s) ACVR2A and ALK5 signaling specifically in myofibers of the subject

13. The method of claim 12, wherein the agent or a combination of agents is/are administered to the subject in an effective amount to increase tricep muscle weight in the subject by at least 3%, at least 4%, or at least 5% relative to a control or baseline.

14. The method of claim 12 or 13, wherein the agent or a combination of agents is/are administered to the subject in an effective amount to increase quadricep muscle weight in the subject by at least 3%, at least 4%, or at least 5% relative to a control or baseline.

15. The method of any one of claims 12-14, wherein the agent or a combination of agents is/are administered to the subject in an effective amount to increase gastrocnemius/plantaris muscle weight in the subject by at least 3%, at least 4%, or at least 5% relative to a control or baseline.

16. A method of increasing muscle weight, reducing body fat content, and/or improving glucose metabolism in a subject, comprising administering to the subject an agent or a combination of agents that inhibit(s) ACVR2B and ALK5 signaling in the subject.

17. The method of claim 16, wherein the agent or a combination of agents is/are administered to the subject in an effective amount to increase muscle weight in the subject by at least 3%, at least 4%, or at least 5% relative to a control or baseline.

18. The method of claim 16 or 17, wherein the agent or combination of agents inhibit(s) ACVR2B and ALK5 signaling by binding to ACVR2B and ALK5.

19. The method of any one of the preceding claims, wherein the agent or combination of agents inhibit(s) ACVR2B and ALK5 signaling specifically in myofibers of the subject

20. The method of claim 19, wherein the agent or a combination of agents is/are administered to the subject in an effective amount to increase tricep muscle weight in the subject by at least 3%, at least 4%, or at least 5% relative to a control or baseline.

21. The method of claim 19 or 20, wherein the agent or a combination of agents is/are administered to the subject in an effective amount to increase quadricep muscle weight in the subject by at least 3%, at least 4%, or at least 5% relative to a control or baseline.

22. The method of any one of claims 19-21, wherein the agent or a combination of agents is/are administered to the subject in an effective amount to increase gastrocnemius/plantaris muscle weight in the subject by at least 3%, at least 4%, or at least 5% relative to a control or baseline.

23. The method of any one of claims 19-22, wherein the agent or a combination of agents is/are administered to the subject in an effective amount to increase pectoralis muscle weight in the subject by at least 3%, at least 4%, or at least 5% relative to a control or baseline.

24. A method of increasing muscle weight, reducing body fat content, and/or improving glucose metabolism in a subject, comprising administering to the subject an agent or a combination of agents that inhibit(s) type I and/or type II receptor signaling in myofibers of the subject.

25. The method of claim 24, wherein the type I receptor is selected from the group consisting of ALK4 and ALK5.

26. The method of claim 25, wherein the agent or combination of agents that inhibit(s) type I receptor signaling binds to ALK4 and/or ALK5.

27. The method of any one of claims 24-26, wherein the type II receptor is selected from the group consisting of ACVR2A, ACVR2B, and TGFβRII.

28. The method of claim 27, wherein the agent or combination of agents that inhibit(s) type I receptor signaling binds to ACVR2A, ACVR2B, and/or TGFβRII.

29. The method of claim 27, wherein the agent or combination of agents that inhibit(s) type I receptor signaling binds to ACVR2A, ACVR2B, and TGFβRII.

30. The method of any one of the preceding claims, wherein the agent or combination of agents inhibit(s) ALK4 and/or ALK5 signaling.

31. The method of any one of the preceding claims, wherein the agent or combination of agents inhibit(s) ACVR2A, ACVR2B, and/or TGFβRII signaling.

32. The method of any one of the preceding claims, wherein the agent or combination of agents inhibit(s) ACVR2A, ACVR2B, and TGFβRII signaling.

33. A method of increasing muscle weight, reducing body fat content, and/or improving glucose metabolism in a subject, comprising administering to the subject an agent or a combination of agents that inhibit(s) (a) TGFβRII and/or (b) TGFβ1, TGFβ2, and/or TGFβ3 signaling in the subject.

34. The method of claim 33, wherein the agent or a combination of agents is/are administered to the subject in an effective amount to increase muscle weight in the subject by at least 3%, at least 4%, or at least 5% relative to a control or baseline.

35. The method of claim 33 or 34, wherein the agent or combination of agents inhibit(s) (a) TGFβRII and/or (b) TGFβ1, TGFβ2 and/or TGFβ3 signaling by binding to (a) TGFβRII and/or (b) TGFβ1, TGFβ2, and/or TGFβ3.

36. The method of any one of claims 33-35 further comprising administering to the subject an agent or a combination of agents that inhibit(s) type I and/or type II receptor signaling in the subject.

37. The method of claim 36, wherein the type I receptor is selected from the group consisting of ALK4 and ALK5.

38. The method of any one of claim 36 or 37, wherein the type II receptor is selected from the group consisting of ACVR2A, ACVR2B, and TGFβRII.

39. The method of any one of claims 36-38, wherein the method comprises administering to the subject an agent or a combination of agents that inhibit(s) signaling through TGFβRII and one or more of the following pairs of receptors:
(a) ALK4 and ALK5;
(b) ACVR2A and AVCR2B;
(c) ALK4 and ACVR2A;
(d) ALK4 and ACVR2B;
(e) ALK5 and ACVR2A; and
(f) ALK5 and ACRV2B.

40. A method of increasing bone mineral density, bone volume, and/or bone density in a subject, comprising administering to the subject an agent or a combination of agents that inhibit(s) ALK4 and/or ALK5 signaling in the subject.

41. The method of claim 40, wherein the agent or combination of agents inhibit(s) ALK4 and ALK5 signaling in the subject.

42. The method of claim 40, wherein the agent or a combination of agents is/are administered to the subject in an effective amount to increase bone mineral density in the subject by at least 3%, at least 4%, or at least 5% relative to a control or baseline.

43. The method of claim 40 or 42, wherein the agent or combination of agents inhibit(s) ALK4 and/or ALK5 signaling by binding to ALK4 and/or ALK5.

44. The method of any one of claims 39-43, wherein the agent or combination of agents inhibit(s) ALK4 and ALK5 signaling specifically in osteoblasts of the subject.

45. The method of claim 44, wherein the agent or a combination of agents is/are administered to the subject in an effective amount to increase total body bone mineral density by at least 3%, at least 4%, or at least 5% relative to a control or baseline.

46. The method of claim 44 or 45, wherein the agent or a combination of agents is/are administered to the subject in an effective amount to increase bone mineral density by at least 3%, at least 4%, or at least 5% at a site selected from the group consisting of lumbar spine, radius, ulna, and pelvis, relative to a control or baseline.

47. The method of any one of claims 40-46, wherein the agent or a combination of agents is/are administered to the subject in an effective amount to increase lumbar spine bone mineral density in the subject by at least 3%, at least 4%, or at least 5% relative to a control or baseline.

48. The method of any one of claims 40-47, wherein the agent or a combination of agents is/are administered to the subject in an effective amount to radius and/or ulna bone mineral density in the subject by at least 3%, at least 4%, or at least 5% relative to a control or baseline.

49. The method of any one of claims 40-48, wherein the agent or a combination of agents is/are administered to the subject in an effective amount to increase pelvis bone mineral density in the subject by at least 3%, at least 4%, or at least 5% relative to a control or baseline.

50. A method of increasing bone mineral density, bone volume, and/or bone density in a subject, comprising administering to the subject an agent or a combination of agents that inhibit(s) ACVR2A and ALK5 signaling in the subject.

51. The method of claim 50, wherein the agent or a combination of agents is/are administered to the subject in an effective amount to increase bone mineral density in the subject by at least 3%, at least 4%, or at least 5% relative to a control or baseline.

52. The method of claim 50 or 51, wherein the agent or combination of agents inhibit(s) ACVR2A and ALK5 signaling by binding to ACVR2A and ALK5.

53. The method of any one of claims 40-52, wherein the agent or combination of agents inhibit(s) ACVR2A and ALK5 signaling specifically in osteoblasts of the subject.

54. The method of claim 53, wherein the agent or a combination of agents is/are administered to the subject in an effective amount to increase total body bone mineral density by at least 3%, at least 4%, or at least 5% relative to a control or baseline.

55. The method of claim 53 or 54, wherein the agent or a combination of agents is/are administered to the subject in an effective amount to increase bone mineral density by at least 3%, at least 4%, or at least 5% at a site selected from the group consisting of lumbar spine, radius, ulna, and pelvis, relative to a control or baseline.

56. The method of any one of claims 50-55, wherein the agent or a combination of agents is/are administered to the subject in an effective amount to increase lumbar spine bone mineral density in the subject by at least 3%, at least 4%, or at least 5% relative to a control or baseline.

57. The method of any one of claims 50-56, wherein the agent or a combination of agents is/are administered to the subject in an effective amount to increase radius and/or ulna bone mineral density in the subject by at least 3%, at least 4%, or at least 5% relative to a control or baseline.

58. The method of any one of claims 50-57, wherein the agent or a combination of agents is/are administered to the subject in an effective amount to increase pelvis bone mineral density in the subject by at least 3%, at least 4%, or at least 5% relative to a control or baseline.

59. A method of increasing bone mineral density, bone volume, and/or bone density in a subject, comprising administering to the subject an agent or a combination of agents that inhibit(s) ACVR2B and ALK5 signaling in the subject.

60. The method of claim 59, wherein the agent or a combination of agents is/are administered to the subject in an effective amount to increase bone mineral density in the subject by at least 3%, at least 4%, or at least 5% relative to a control or baseline.

61. The method of claim 59 or 60, wherein the agent or combination of agents inhibit(s) ACVR2B and ALK5 signaling by binding to ACVR2B and ALK5.

62. The method of any one of claims 40-61, wherein the agent or combination of agents inhibit(s) ACVR2B and ALK5 signaling specifically in osteoblasts of the subject.

63. The method of claim 62, wherein the agent or a combination of agents is/are administered to the subject in an effective amount to increase total body bone mineral density by at least 3%, at least 4%, or at least 5% relative to a control or baseline.

64. The method of claim 62 or 63, wherein the agent or a combination of agents is/are administered to the subject in an effective amount to increase bone mineral density by at least 3%, at least 4%, or at least 5% at a site selected from the group consisting of lumbar spine, radius, ulna, and pelvis, relative to a control or baseline.

65. The method of any one of claims 62-64, wherein the agent or a combination of agents is/are administered to the subject in an effective amount to increase lumbar spine bone mineral density in the subject by at least 3%, at least 4%, or at least 5% relative to a control or baseline.

66. The method of any one of claims 62-65, wherein the agent or a combination of agents is/are administered to the subject in an effective amount to increase radius and/or ulna bone mineral density in the subject by at least 3%, at least 4%, or at least 5% relative to a control or baseline.

67. The method of any one of claims 62-66, wherein the agent or a combination of agents is/are administered to the subject in an effective amount to increase pelvis bone mineral density in the subject by at least 3%, at least 4%, or at least 5% relative to a control or baseline.

68. The method of any one of claims 62-67, wherein the agent or a combination of agents is/are administered to the subject in an effective amount to increase vertebrae bone mineral density in the subject by at least 3%, at least 4%, or at least 5% relative to a control or baseline.

69. A method of increasing bone mineral density, bone volume, and/or bone density in a subject, comprising administering to the subject an agent or a combination of agents that inhibit(s) type 1 and/or type 2 receptor signaling in osteoblasts of the subject.

70. The method of claim 69, wherein the type 1 receptor is selected from the group consisting of ALK4 and ALK5.

71. The method of claim 70, wherein the agent or combination of agents that inhibit(s) type 1 receptor signaling binds to ALK4 and/or ALK5.

72. The method of any one of claims 69-71, wherein the type 2 receptor is selected from the group consisting of ACVR2A, ACVR2B, and TGFβRII.

73. The method of claim 72, wherein the agent or combination of agents that inhibit(s) type 1 receptor signaling binds to ACVR2A, ACVR2B, and/or TGFβRII.

74. The method of claim 72, wherein the agent or combination of agents that inhibit(s) type 1 receptor signaling binds to ACVR2A, ACVR2B, and TGFβRII.

75. The method of any one of claims 40-74, wherein the agent or combination of agents inhibit(s) ALK4 and/or ALK5 signaling.

76. The method of any one of claims 40-75, wherein the agent or combination of agents inhibit(s) ACVR2A, ACVR2B, and/or TGFβRII signaling.

77. The method of any one of claims 40-76, wherein the agent or combination of agents inhibit(s) ACVR2A, ACVR2B, and TGFβRII signaling.

78. A method of increasing bone mineral density, bone volume, and/or bone density in a subject, comprising administering to the subject an agent or a combination of agents that inhibit(s) (a) TGFβRII and/or (b) TGFβ1, TGFβ2, and/or TGFβ3 signaling in the subject.

79. The method of claim 78, wherein the agent or a combination of agents is/are administered to the subject in an effective amount to increase bone mineral density in the subject by at least 3%, at least 4%, or at least 5% relative to a control or baseline.

80. The method of claim 79, wherein the agent or a combination of agents is/are administered to the subject in an effective amount to increase total body bone mineral density by at least 3%, at least 4%, or at least 5% relative to a control or baseline.

81. The method of claim 79 or 80, wherein the agent or a combination of agents is/are administered to the subject in an effective amount to increase bone mineral density by at least 3%, at least 4%, or at least 5% at a site selected from the group consisting of lumbar spine, radius, ulna, and pelvis, relative to a control or baseline.

82. The method of any one of claims 79-81, wherein the agent or a combination of agents is/are administered to the subject in an effective amount to increase lumbar spine bone mineral density in the subject by at least 3%, at least 4%, or at least 5% relative to a control or baseline.

83. The method of any one of claims 79-82, wherein the agent or a combination of agents is/are administered to the subject in an effective amount to increase radius and/or ulna bone mineral density in the subject by at least 3%, at least 4%, or at least 5% relative to a control or baseline.

84. The method of any one of claims 79-83, wherein the agent or a combination of agents is/are administered to the subject in an effective amount to increase pelvis bone mineral density in the subject by at least 3%, at least 4%, or at least 5% relative to a control or baseline.

85. The method of any one of claims 78-84, wherein the agent or combination of agents inhibit(s) (a) TGFβRII and/or (b) TGFβ1, TGFβ2 and/or TGFβ3 signaling by binding to (a) TGFβRII and/or (b) TGFβ1, TGFβ2, and/or TGFβ3.

86. The method of any one of claims 78-85 further comprising administering to the subject an agent or a combination of agents that inhibit(s) type 1 and/or type 2 receptor signaling in the subject.

87. The method of claim 86, wherein the type 1 receptor is selected from the group consisting of ALK4 and ALK5.

88. The method of claim 86 or 87, wherein the type 2 receptor is selected from the group consisting of ACVR2A, ACVR2B, and TGFβRII.

89. The method of any one of claims 86-88, wherein the method comprises administering to the subject an agent or a combination of agents that inhibit(s) signaling through TGFβRII and one or more of the following pairs of receptors:
(a) ALK4 and ALK5;
(b) ACVR2A and AVCR2B;
(c) ALK4 and ACVR2A;
(d) ALK4 and ACVR2B;
(e) ALK5 and ACVR2A; and
(f) ALK5 and ACRV2B.
